(19) United States
(12) Patent Application Publication
(10) Pub. No.: US 2022/0340954 A1
(43) Pub. Date: Oct. 27, 2022

(54) METHOD FOR SEPARATING NUCLEIC ACID MOLECULES BY SIZE

(71) Applicant: QIAGEN GmbH, Hilden (DE)

(72) Inventors: Lothar BREITKOPF, Hilden (DE); Jasper STROEDER, Hilden (DE)

(21) Appl. No.: 17/620,615

(22) PCT Filed: Jun. 26, 2020
(86) PCT No.: PCT/EP2020/068077

§ 371 Date: Dec. 17, 2021

(30) Foreign Application Priority Data
Jun. 28, 2019 (EP) 19183295.5

(51) Int. Cl. C12Q 1/6806; C12N 15/10

(52) U.S. Cl. CPC C12Q 1/6806; C12N 15/1065

(57) ABSTRACT

The present invention provides a poly(alkylene oxide) polymer based size selective nucleic acid enrichment method for enriching target nucleic acid molecules from a nucleic acid containing sample which comprises target nucleic acid molecules and non-target nucleic acid molecules, wherein the target nucleic acid molecules are longer than the non-target nucleic acid molecules, the method comprising (a) preparing a binding mixture comprising—the nucleic acid containing sample, —a poly(alkylene oxide) polymer and—a salt and binding nucleic acid molecules to a solid phase which comprises a functional group, preferably carboxylated magnetic particles, wherein the bound nucleic acid molecules comprise target nucleic acid molecules; (b) preferably separating the solid phase with the bound nucleic acid molecules from the remaining sample; (c) contacting the solid phase with the bound nucleic acid molecules at least once with a reagent composition comprising a poly(alkylene oxide) polymer and a salt to selectively elute non-target nucleic acid molecules, wherein the concentration (w/v) of the poly(alkylene oxide) polymer in the reagent composition of step (c) is lower than the concentration (w/v) of the poly(alkylene oxide) polymer in the binding mixture of step (a); (d) optionally washing the bound target nucleic acid molecules; and (e) eluting the bound target nucleic acid molecules from the solid phase. Said method allows the size selective purification of target DNA molecules and is particularly suitable for sequencing applications. It is more time- and cost efficient than prior art methods and provides excellent purification results. Moreover, further methods and kits are provided.

### FIELD OF INVENTION

The present invention provides a method for the size selective enrichment of nucleic acid molecules. The method of the present invention is particularly useful for enriching target DNA molecules having a desired minimum length for preparing a sequencing library. Furthermore, a kit is provided that can be used for the size selective enrichment of nucleic acid molecules.

### BACKGROUND OF THE INVENTION

Different methods for isolating nucleic acids are well-known in the prior art. Such methods involve separating nucleic acids of interest from other sample components, such as for example protein contaminations or potentially other nucleic acids, also often referred to as non-target nucleic acids. If it is intended to isolate a specific nucleic acid of interest from other nucleic acids the separation process is usually based on differences in parameters of the target and the non-target nucleic acid such as for example their topology (for example super-coiled DNA from linear DNA), their size (length) or chemical differences (e.g. DNA from RNA) and the like.

For certain applications differences in the size is an important criterion to distinguish target nucleic acids from non-target nucleic acids. E.g. size selective fractionation of DNA is an important step in the library construction for next generation sequencing (NGS) applications. Different NGS technologies and methods exist such as pyrosequencing, sequencing by synthesis or sequencing by ligation. Most NGS platforms share a common technological feature, namely the massively parallel sequencing of clonally amplified or single DNA molecules that are spatially separated in a flow cell or by generation of an oil-water emulsion. In NGS, sequencing is performed by repeated cycles of polymerase-mediated nucleotide extensions or, in one format, by iterative cycles of oligonucleotide ligation. As a massively parallel process, NGS generates hundreds of megabases to gigabases of nucleotide-sequence output in a single instrument run, depending on the platform. NGS technologies have become a major driving force in genetic research. Several NGS technology platforms have found widespread use and include, for example, the following NGS platforms: Roche/454, Illumina Solexa Genome Analyzer, the Applied Biosystems SOLiD™ system, Ion Torrent™ semiconductor sequence analyzer, PacBio® real-time sequencing and Helicos™ Single Molecule Sequencing (SMS). NGS technologies, NGS platforms and common applications/fields for NGS technologies are e.g. reviewed in Voelkerding et al (Clinical Chemistry 55:4 641-658, 2009), Metzker (Nature Reviews/Genetics Volume 11, January 2010, pages 31-46), Goodwin et al. (Nature Reviews, June 2016, Vol. 17: pp. 333-351), Yohe et al. (Arch Pathol Lab Med, November 2017, Vol. 141: pp. 1544-1557) and Masoudi-Nejad, Chapter 2 “Emergence of Next-Generation Sequencing in “Next Generation Sequencing and Sequence Assembly” SpringerBriefs in Systems Biology, 2013.

NGS technology platforms have in common that they require the preparation of a sequencing library which is suitable for massive parallel sequencing. Examples of such sequencing libraries include fragment libraries, mate-paired libraries or barcoded fragment libraries. Most platforms adhere to a common library preparation procedure with minor modifications before a “run” on the instrument. This procedure includes, if required, fragmenting the DNA (which may also be obtained from cDNA), e.g. by mechanical shearing or enzymatic fragmentation followed by DNA repair and end polishing (blunt end or A overhang) and, finally, platform-specific adapter ligation. The preparation and design of such sequencing libraries is well-known and also described in the above review articles. In order to analyze data using Next Generation Sequencing, DNA molecules of interest need to be ligated to sequencing adapters prior to library amplification and sequencing in a step commonly referred to as library preparation.

In order to ensure high quality sequencing data, efficient library preparation-methods are needed. Furthermore, to reduce the background in the sequencing reads, it is important to remove DNA contaminants that might be present in the sequence library as a result of the library preparation. An important example of such DNA contaminants are adapter monomers and adapter-adapter ligation products such as adapter dimers that are often present in the sequencing library after adapter ligation. To ensure efficient adapter ligation, adapters are used in excess during adapter ligation. Thus, after adapter ligation, unligated adapter monomers and adapter-adapter ligation products such as adapter dimers are present in addition to the adapter ligated DNA molecules. It is important to remove unligated adapter monomers and adapter-adapter ligation products from the adapter ligated DNA molecules to ensure minimal background amplification during sequencing. Otherwise, they will use up sequencing capacity, thereby diminishing the power available to investigate sequences of interest. Unligated adapter monomers and adapter-adapter ligation products are usually removed by a size selective purification of the larger adapter ligated DNA molecules, which contain the DNA fragments to be sequenced.

Several approaches were developed in the prior art in order to isolate DNA of a specific target size, respectively of a specific target size range. These size selection methods can be used in order to remove adapter dimers and monomers, as these DNA contaminations have a size that is smaller than the adapter ligated DNA molecules. A classic method for isolating DNA of a target size involves the separation of the DNA in a gel, cutting out the desired gel band(s) and then isolating the DNA of the target size from the gel fragment(s). Respective gel based size selection methods are often recommended in many next generations sequencing library preparation protocols in order to remove adapter monomers and dimers.

However, respective methods are time consuming, as the portion of the gel containing the nucleic acids of interest must be manually cut out and then treated to degrade the gel or otherwise extract the DNA of the target size from the gel slice.

Another widely used technology is the size selective precipitation with a poly(alkylene oxide) polymer containing buffer, in particular polyethylene glycol based buffers (Lis and Schleif Nucleic Acids Res. 1975 March; 2(3):383-9) or the binding/precipitation on carboxyl-functionalized beads (DeAngelis et al, Nuc. Acid. Res. 1995, Vol 23(22), 4742-3; U.S. Pat. No. 5,898,071 und U.S. Pat. No. 5,705,628, commercialized by Beckman-Coulter (AmPure XP; SPRIselect) and U.S. Pat. No. 6,534,262).

The most common way of adapter removal as reflected in the AmPure XP product utilizes carboxylated magnetic particles that bind DNA fragments samples in the presence of a polyethylene glycol containing buffer and show a PEG concentration dependent size selectivity for DNA. The AmPure XP product is widely used and thus can be perceived as standard reference product. The DNA libraries are bound to magnetic particles under conditions that favor the binding of larger DNA fragments and thus leave short DNA fragments behind. The samples are then washed, dried and eluted. However, since adapter removal after one round of size selection is often incomplete, another round of (selective) bind, wash, elute usually follows, thereby doubling the time and effort needed to ensure efficient adapter removal. The principle work-flow is as follows:
binding of sample in the presence of adjusted PEG concentration, leaving the adapter DNA in solution due to size-selectivity of the binding buffer
Wash the beads twice in ethanol
Dry the beads
Elute in water
Second round of sample binding through the addition of PEG buffer
Wash the beads twice in ethanol
Dry the beads
Elution in water/TE.

Also other established size-selection protocols require two/multiple subsequent adapter removal steps in order to thoroughly remove all sequencing adapters/dimers efficiently (see e.g. QIAseq Targeted DNA Panels (QIAGEN), QIAseq FX DNA Library Kit (QIAGEN) or QIAseq CfDNA Library Kit (QIAGEN)).

Thus, even though polyethylene glycol based size selection methods are established as gold standard for size selective DNA enrichment, in particular for adapter removal in NGS applications, there is a need for improved methods. In particular, there is a need for more simple, time and cost-efficient methods for enriching nucleic acid molecules of a specific minimum size that can be integrated into existing next generation sequencing library preparation protocols.

It is an object of the present invention to provide a method for enriching nucleic acid molecules of a target size from a nucleic acid containing sample comprising nucleic acid molecules of different sizes. In particular, it is the object of the present invention to provide a more time- and cost efficient poly(alkylene oxide) polymer based size selective DNA enrichment method. Furthermore, it is an object to provide a fast, simple and cost-efficient method for removing unligated adapter monomers and adapter dimers from adapter ligation samples, in particular adapter ligation samples obtained in the preparation of a sequencing library.

### SUMMARY OF THE INVENTION

The present invention is based on the established size selective nucleic acid isolation technology, wherein at least one poly(alkylene oxide) polymer, such as a polyethylene glycol, is used in order to bind target nucleic acid molecules having a size above a certain cut-off value from a nucleic acid containing sample to a functionalized solid phase, such as carboxyl group functionalized magnetic particles. As disclosed herein, the binding may be size-selective. The target nucleic acid molecules that are bound to the solid support can be recovered and removed from the remaining sample. As discussed in the background, the poly(alkylene oxide) polymer based size selective methods known in the prior art have the drawback that several rounds (cycles) of size selection (“bind-wash-elute”) are usually required in order to efficiently remove smaller non-target nucleic acid molecules, such as adapter monomers or adapter-adapter ligation products, from the target nucleic acid molecules.

The present disclosure provides a size selective nucleic acid enrichment method that is faster and more cost-efficient compared to the prior art. The method is particularly suitable for enriching adapter ligated DNA molecules as target DNA molecules from an adapter ligation sample, while efficiently removing adapter monomers and adapter-adapter ligation products. After preferably size-selective binding of the target nucleic acid molecules to the solid phase, the present method introduces a size-selective elution step into the workflow. The solid phase with the bound nucleic acid molecules is treated with a reagent composition that comprises a poly(alkylene oxide) polymer and a salt (preferably a non-chaotropic salt such as an alkali metal salt). Thereby, conditions are established that favor the binding of the longer target DNA fragments to the functionalized solid phase (e.g. carboxylated magnetic particles) which therefore remain bound to the solid phase, while unwanted shorter fragments that were besides the used size-selective binding conditions nevertheless bound to the solid phase, such as adapter monomers or adapter dimers, are eluted due to the treatment.

Performing this intermediate treatment step enables the effective removal of e.g. excessive adapters from the processed sample and makes a second round/cycle of “bind-wash-elute”, as it is performed in prior art methods, unnecessary. E.g. instead of performing two (or more) subsequent rounds of adapter removal, one round of adapter removal with an additional size selective-processing step between the binding and elution step is sufficient for the complete removal of sequencing adapters. Based on the poly(alkylene oxide) polymer concentration used during this size selective elution step, non-target DNA fragments below a certain cut-off are eluted, while larger target DNA fragments remain bound to the solid phase and can be further processed by preferably washing the bound target DNA molecules and subsequent elution. The overall process is therefore considerably simplified and improved compared to prior art methods.

Furthermore, the present technology significantly reduces the required amount of solid phase (e.g. magnetic particles comprising carboxyl groups), requires less reagents and is faster than prior art methods. In addition, as shown in the examples, the present method provides at least the same degree of efficiency, e.g. adapter removal, as conventional, more time and cost consuming protocols. In addition, the present method can be easily adapted for simple automation devices without the need for pipetting units.

Thereby, a highly efficient poly(alkylene oxide) polymer based size-selective nucleic acid enrichment method is provided that is more time- and cost-efficient than prior art methods. The present method is faster and cheaper than prior art methods because it requires less material and lab equipment in order to efficiently remove non-target nucleic acid molecules having a size below the cut-off value (such as adapter monomers and adapter-adapter ligation products) and purify target nucleic acid molecules (e.g. adapter ligated DNA molecules). Furthermore, it can easily be automated and the library yields were often better, compared to standard procedures, as is demonstrated in the examples. The present invention therefore provides an important improvement compared to common size selective nucleic acid enrichment methods such as the commonly used methods for adapter removal.

According to a first aspect of the present disclosure a poly(alkylene oxide) polymer based size selective nucleic acid enrichment method is provided for enriching target nucleic acid molecules from a nucleic acid containing sample which comprises target nucleic acid molecules and non-target nucleic acid molecules, wherein the target nucleic acid molecules are longer than the non-target nucleic acid molecules, the method comprising
(a) preparing a binding mixture comprising
   the nucleic acid containing sample,
   a poly(alkylene oxide) polymer and
   a salt
   and binding nucleic acid molecules to a solid phase which comprises a functional group, preferably carboxylated magnetic particles, wherein the bound nucleic acid molecules comprise target nucleic acid molecules;
(b) preferably separating the solid phase with the bound nucleic acid molecules from the remaining sample;
(c) contacting the solid phase with the bound nucleic acid molecules at least once with a reagent composition comprising a poly(alkylene oxide) polymer and a salt to selectively elute non-target nucleic acid molecules, wherein preferably, the concentration (w/v) of the poly(alkylene oxide) polymer in the reagent composition of step (c) is lower than the concentration (w/v) of the poly(alkylene oxide) polymer in the binding mixture of step (a);
(d) optionally washing the bound target nucleic acid molecules; and
(e) eluting the bound target nucleic acid molecules from the solid phase.

Step (c) allows to efficiently remove small non-target nucleic acid molecules that were bound in step (a) by performing a size-selective elution step which preferentially elutes the small non-target nucleic acid molecules while the target nucleic acids remain bound to the solid phase. The present method is particularly suitable for the size selective purification of target DNA molecules, such as adapter ligated DNA molecules as target DNA molecules from an adapter ligation sample and for removing adapter monomers and adapter-adapter ligation products as non-target DNA molecules.

According to a second aspect of the present disclosure a method for preparing a sequencing library that is suitable for massive parallel sequencing is provided, wherein said method comprises
A) optionally fragmenting DNA and optionally end repairing DNA fragments to provide a sample comprising blunt end DNA fragments of different sizes;
B) performing an adapter ligation step to provide a sample comprising double-stranded DNA molecules that are flanked 5′ and/or 3′ by adapters;
C) separating adapter ligated double-stranded DNA molecules from unligated adapter monomers and adapter-adapter ligation products based on the larger size of the adapter ligated double stranded DNA molecules using the method according to the first aspect; and
D) optionally amplifying adapter ligated DNA molecules.

As discussed herein the present size selective nucleic acid enrichment method is particularly suitable for separating adapter ligated double-stranded DNA molecules from unligated adapter monomers and adapter-adapter ligation products based on the larger size of the adapter ligated double stranded DNA and can be easily integrated into next generation sequencing work-flows.

According to a third aspect of the present disclosure a kit is provided for the size selective enrichment of target nucleic acid molecules having a size above a desired cut-off value from a nucleic acid containing sample, comprising
(a) a binding reagent comprising at least one poly(alkylene oxide) polymer and at least one salt;
(b) magnetic particles comprising a functional group, preferably a carboxyl group;
(c) a reagent comprising at least one poly(alkylene oxide) polymer and at least one salt and/or a dilution reagent for preparing the reagent (c) by combining the dilution reagent with the binding reagent;
(d) optionally at least one washing solution; and
(e) optionally an elution solution,
   wherein the concentration of the poly(alkylene oxide) polymer in the binding reagent (a) is higher than the concentration of the poly(alkylene oxide) polymer in the reagent (c). A respective kit can be advantageously used in conjunction with and for performing the method according to the first aspect of the present disclosure.

The fourth aspect of the present disclosure pertains to the use of a kit according to the third aspect of the present disclosure in a method according to the first aspect of the present

### DISCLOSURE

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only. Various changes and modifications within the spirit and scope of the disclosed invention will become readily apparent to those skilled in the art from reading the following.

### BRIEF DESCRIPTION OF THE FIGURES

FIG. 1: shows an electropherogram of the starting material 1 before adapter removal as analyzed on a bioanalyzer. Peaks resulting from adapter dimers (approx. 120-130 bp), additional minor impurities (150-200 bp) and library (approx. 300 bp-1800 bp) were identified.

FIG. 2: shows an electropherogram of the purified eluate of starting material 1 after adapter removal with AMPure beads using two rounds of adapter removal as analyzed on a bioanalyzer. Only library sample (300 bp-1800 bp) was identified.

FIG. 3: shows an electropherogram of the result of the adapter-enrichment PCR using the purified eluate (FIG. 2) as template. 12 enrichment cycles of PCR were performed. The amplified sample was loaded onto an Agilent Bioanalyzer to visualize the presence of amplified sequencing adapters. Amplified sequencing adapters at around 120 bp were detectable to a minor amount, demonstrating minute remaining levels of adapter dimers in the purified eluate.

FIG. 4: shows an electropherogram of starting material 2 before adapter removal as analyzed on a bioanalyzer. Peaks resulting from adapter dimers (approx. 120-130 bp), additional impurities (150-200 bp) and library (approx. 300 bp-1800 bp) were identified.

FIG. 5: shows an electropherogram of the purified eluate of starting material 2 after adapter removal with AMPure beads using two rounds of adapter removal as analyzed on a bioanalyzer. Only library sample (300 bp-1800 bp) was identified.

FIG. 6a: shows an electropherogram of the spin-column purified DNA isolated from the supernatant of the 1^{st} binding step according to the method of the prior art. Adapter dimers but also DNA library were not bound to the AMPure beads. (1:37 dilution of the analyzed sample, concentration without dilution 56.06 ng/μl).

FIG. 6b: shows an electropherogram of the spin-column purified DNA isolated from the supernatant of the 2^{nd} binding step according to the method of the prior art. Adapter dimers but also DNA library were excluded from binding to the AMPure beads. (1:8 dilution of the analyzed sample, concentration without dilution 10.14 ng/μl).

FIG. 7: shows an electropherogram of the purified eluate of starting material 1 after adapter removal as analyzed on a bioanalyzer. Peaks resulting from adapter dimers were removed and only library sample was identified.

FIG. 8: shows an electropherogram of the spin-column purified DNA isolated from the supernatant obtained after binding step (a) of the present method. Specifically, adapter dimers and some larger impurities were not bound by the carboxylated beads, while virtually the entire library bound to the beads.

FIG. 9: shows an electropherogram of the spin-column purified DNA isolated from the supernatant obtained after the 1^{st} size-selective elution step in the method according to the invention. Specifically, adapter dimers and larger impurities of 200 bp were selectively eluted from the beads, while elution of library sample (300 bp-1800 bp) from the beads was kept to a minimum.

FIG. 10: shows an electropherogram of the result of the adapter-enrichment PCR. The presence of amplified sequencing adapters at around 120 bp could be shown, however, demonstrating only minute remaining levels of adapter dimers in the adapter-removed, purified eluate.

FIG. 11: shows an electropherogram of the purified eluate of starting material 2 after adapter removal as analyzed on a bioanalyzer.

FIG. 12: shows an electropherogram of the spin-column purified DNA isolated from the supernatant of the binding step (a) provided in the present method. Specifically, adapter dimers and some larger impurities were excluded from binding to the carboxylated beads, while virtually the entire library bound to the beads.

FIG. 13: shows an electropherogram of the spin-column purified DNA isolated from the supernatant of the 1^{st} size-selective elution step in the method according to the invention. Specifically, adapter dimers and larger impurities of 200 bp were selectively eluted from the beads, while elution of library sample (300 bp-1800 bp) from the beads was kept to a minimum.

FIG. 14: shows an electropherogram of the spin-column purified DNA isolated from the supernatant of the 2^{nd} size-selective elution step in the method according to the invention. Specifically, adapter dimers and larger impurities of 200 bp were selectively eluted from the beads, while elution of library sample (300 bp-1800 bp) from the beads was kept to a minimum.

FIG. 15: shows an electropherogram of the adapter-removed, purified eluate of starting material 1 that was purified using the automated method according to the invention and analyzed on a bioanalyzer. Essentially only library sample (300 bp-1800 bp) was identified.

FIG. 16: shows an electropherogram of the spin-column purified DNA isolated from the supernatant of the binding step of the automated method according to the invention. Specifically adapter dimers were excluded from binding to the beads, while virtually the entire library had bound to the beads.

FIG. 17: shows an electropherogram of the spin-column purified DNA isolated from the supernatant of the 1^{st} size-selective elution step of the automated method according to the invention. Specifically adapter dimers were eluted from the beads, while elution of library sample was kept to a minimum (1:5 dilution of the analyzed sample, concentration without dilution 3.95 ng/μl).

FIG. 18: shows an electropherogram of the spin-column purified DNA isolated from the supernatant of the 2^{nd} size-selective elution step of the automated method according to the invention. Specifically NGS adapter dimers and larger impurities of 200 bp were eluted from the beads, but only little elution of library occurred (no dilution of the analyzed sample, concentration 1.33 ng/μl).

FIG. 19: shows an electropherogram of the result of the adapter-enrichment PCR performed with the adapter-removed, purified eluate of starting material 1 (FIG. 15) as template. The presence of sequencing adapters at around 120 bp was minimal, demonstrating only minute amounts of remaining adapter dimers in the purified sample.

FIG. 20: shows an electropherogram of the adapter-removed, purified eluate of starting material 2 that was obtained using the automated method according to the invention and analyzed on a bioanalyzer. Essentially only library sample was identified. Adapter-dimers were efficiently removed.

FIG. 21: shows an electropherogram of the spin-column purified DNA isolated from the supernatant provided in the binding step of the automated method according to the invention. Specifically adapter dimers and additional impurities were not bound to the beads, while virtually the entire library had bound to the beads.

FIG. 22: shows an electropherogram of the spin-column purified DNA isolated from the supernatant of the 1^{st} size-selective elution step of the automated method according to the invention. Specifically adapter dimers and additional impurities were eluted from the beads, while elution of library sample was kept low (1:5 dilution of the analyzed sample, concentration without dilution 3.714 ng/μl).

FIG. 23: shows an electropherogram of the spin-column purified DNA isolated from the supernatant of the 2^{nd} size-selective elution step of the automated method according to the invention. Specifically adapter dimers and additional impurities were eluted from the beads, while elution of library sample was kept low (1:5 dilution of the analyzed sample, concentration without dilution 1.572 ng/μl).

FIG. 24: is a bar graph based figure showing the DNA yield measured in the eluates after performing the different adapter removal workflows. Sample purification was performed in duplicate (A and B). DNA concentration in the final eluates was determined by fluorescent measurement on a Qubit and use of the Agilent High Sensitivity Chip. Library recovery was improved using the manual or automated inventive method over the prior art reference method.

FIG. 25: Sample purification and analysis was performed in duplicates (A: left bar, B: right bar). For input and final yields, the total amount of DNA used or finally recovered, respectively, are indicated. DNA contained in the supernatant (SN) of the indicated purification steps was analyzed by spin-column cleanup of the supernatants followed by DNA measurements of one manually and both automated purified samples using a bioanalyzer. DNA measurements were performed with the Agilent BioAnalyzer (High Sensitivity DNA Analysis Kit, Agilent Genomics). SN 2^{nd} binding refers to the supernatant of the binding step of the second “bind-wash-elute” cycle (AMPure), while SN selective elution refers to the supernatant of size-selective elution step (c) of the inventive method which was performed twice. As the final DNA yields show, the library recovery and thus the target DNA recovery was improved when using the method according to the invention.

FIG. 26: shows an electropherogram of starting material 3 prior to adapter removal as analyzed on a bioanalyzer. Peaks resulting from adapter dimers (approx. 120-130 bp), additional impurities and library (approx. 220-1400 bp) were identified.

FIG. 27: shows an electropherogram of the adapter-removed, purified eluate of starting material 3 (pH 4.6). Only library sample was identified.

FIG. 28: shows an electropherogram of the spin-column purified DNA isolated from the supernatant of the binding step (pH 4.6). Specifically, adapter dimers were excluded from binding to the beads and thus remained in the supernatant. The library sample was efficiently bound to the beads.

FIG. 29: shows an electropherogram of the spin-column purified DNA isolated from the supernatant of the 1^{st} size-selective elution step (c) (pH 4.6). Specifically adapter dimers and impurities were eluted from the beads, while elution of library sample was kept to a minimum (1:5 dilution of the sample).

FIG. 30: is a bar graph based figure showing the amount of DNA found at each step of the adapter removal protocol performed with starting material 3 using either PEG-buffers at pH 4.6 (black bars) or at pH 8.2 (white bars). Data are mean values of 2 experiments each. For input and final yields the total amount of DNA used or finally recovered, respectively, are indicated. DNA contained in the supernatant of the purification steps (1^{st} binding, 1^{st} selective elution, 2^{nd} selective elution) was analyzed by spin-column cleanup of the supernatants followed by DNA measurements of the spin-column purified DNA using a bioanalyzer. Library recovery was similar irrespective of the pH used in the PEG-containing buffers. Hence, the PEG-buffers can be used/worked over a broad pH range.

FIG. 31: shows an electropherogram of starting material 4 prior to adapter removal. Peaks resulting from adapter dimers (120-130 bp), additional impurities and library (300 bp) were identified.

FIG. 32: shows an electropherogram of the purified eluate of starting material 4 after adapter removal. The pH during binding step (a) and size-selective elution step (c) was pH 4.6. Only library sample was identified.

FIG. 33: shows an electropherogram of the spin-column purified DNA isolated from the supernatant of the binding step (pH 4.6). Adapter dimers were efficiently excluded from binding to the beads, while virtually all library sample was bound to the beads and therefore, was not found in the supernatant.

FIG. 34: shows an electropherogram of the spin-column purified DNA isolated from the supernatant of the first size-selective elution step (c) (pH 4.6). Adapter dimers and impurities were selectively eluted from the beads and are thus found in the spin-column purified DNA, while elution of library sample and thus target DNA during size-selective elution step (c) was kept to a minimum (1:5 dilution of the sample).

FIG. 35: is a bar graph based figure showing the amount of DNA found at each step of the adapter removal protocol performed with starting material 4 using either buffers at pH 4.6 (black bars) or at pH 8.2 (white bars). Data are mean values of 2 experiments each. For input and final yields the total amount of DNA used or finally recovered, respectively, are indicated. DNA contained in the supernatant of the purification steps (1^{st} binding, 1^{st} selective elution, 2^{nd} selective elution) was analyzed by spin-column cleanup of the supernatants followed by DNA measurements of the spin-column purified DNA using a bioanalyzer. Library recovery was again similar irrespective of the pH used in the PEG-containing buffers.

FIG. 36: shows an electropherogram of the starting material used in Example 6 before adapter removal. Peaks representing adapter dimers (approx. 130 bp, 40 s) and library (approx. 300 bp-1800 bp, 55-75 s) were identified.

FIG. 37: shows an overlay of electropherograms of the spin column purified DNA from the supernatant. The PEG concentrations of the buffers used for the binding are indicated in the legend. 1.1× sample volume of PEG-containing buffer was used to mediate the binding of nucleic acids to the magnetic carboxylated beads.

FIG. 38: shows an overlay of electropherograms of the spin column purified DNA from the supernatant of the 1^{st} size-selective elution step with different mixing ratio of 30% PEG 8000 (w/v) and buffer TE. The ratios of PEG-buffer to TE buffer that were used for the size-selective elution step are written in the legend.

FIG. 39: shows overlaid electropherograms of the spin column purified DNA from the supernatant of the 1^{st} size-selective elution step. The PEG-buffer that was used for the dilution contained 15% PEG (w/v). The ratios of PEG-buffer to TE buffer that were used for the size-selective elution are written in the legend.

FIG. 40: shows an overlay of electropherograms after final elution of DNA from carboxylated beads following adapter removal by size-selective elution with various dilutions of a PEG-buffer comprising 30% PEG before being diluted. The dilution factor of PEG-buffer to TE that was used during the size-selective elution step can be found in the legend. A higher dilution factor indicates a higher PEG concentration during this step. Notable are the differences in the adapter-dimer peak (40 s) for the various dilutions during the size-selective elution.

FIG. 41: shows an overlay of electropherograms after final elution of DNA from carboxylated beads following adapter removal by size selective elution with various dilution factors of PEG-buffer comprising 15% PEG before being diluted. The dilution factor of PEG-buffer to TE that was used during the size-selective elution step is indicated in the legend. A higher dilution factor indicates a higher PEG concentration during this step. Notable is the absence of the adapter-dimer peak (40 s) in the eluates.

FIG. 42: shows an overlay of electropherograms of the spin column purified DNA from the supernatant of the binding step. 1.1×amount of sample volume (1.1 volume PEG buffer to 1 volume sample) was used to mediate the binding of DNA onto magnetic beads. Please note the absence of the library DNA peak (compared to the starting material from FIG. 36, 50-75 s) while a strong adapter peak (˜130 bp, 40 s) remained in the supernatant.

FIG. 43: shows overlaid electropherograms from spin-column purified supernatant derived from the 1^{st} size-selective elution step with the PEG-buffer containing PEG 20000 in various dilutions. The ratios of PEG-buffer to TE buffer that were used for the size-selective elution (and resulted in the reagent composition) are written in the legend.

FIG. 44: shows overlaid electropherograms from spin-column purified supernatant derived from the 1^{st} size-selective elution step with the PEG-buffer containing PEG 3000 in various dilutions. The ratios of PEG-buffer to TE buffer that were used for the size-selective elution (and resulted in the reagent composition) are written in the legend.

FIG. 45: shows an overlay of electropherograms of the DNA present in final eluates after size-selective elution with different mixing ratios of 20% PEG 20000. The dilution factor of PEG-buffer to TE that was used during the size-selected elution step is indicated in the legend. The adapter-peak (˜130 bp, 40s) that was present in the starting material is absent in the eluates indicating that the adapters were efficiently removed.

FIG. 46: shows an overlay of electropherograms of the DNA present in final eluates after size-selective elution with different mixing ratios of 20% PEG 3000. The dilution factor of PEG-buffer to TE that was used during the size-selected elution step is indicated in the legend. The adapter-peak (˜130 bp, 40s) that was present in the starting material is absent in the eluates indicating that the adapters were efficiently removed.

FIG. 47: displays the DNA concentration of the final eluates, determined by measuring with Qubit high sense dsDNA Kit, after the removal of adapter dimers from the sample. The graph illustrates the working range for the size selective elution step in order to remove adapters from the library analysed while recovering the library DNA. Note, that the working range depends on the library/target nucleic acid to be purified. The decrease in library yield represents a loss of library prior to the final elution step that can occur during the size selective elution step (“wash”). Under the experimental set up chosen, a loss of library was observed at dilutions between dilution factors of 0.9× and 0.7× when using a lower-molecular PEG (PEG 3000). In contrast library loss was only observed at dilution factors <0.7× when using higher molecular PEG (8000, 20000).

### DETAILED DESCRIPTION OF THE INVENTION

The present invention provides a very efficient poly(alkylene oxide) polymer based size selective method for enriching target nucleic acid molecules by size from a nucleic acid containing sample comprising nucleic acid molecules of different sizes.

### Method for Enriching Target Nucleic Acid Molecules Having a Size Above a Certain Cut-Off Value from a Nucleic Acid Containing Sample

According to a first aspect of the present disclosure a poly(alkylene oxide) polymer based size selective nucleic acid enrichment method is provided for enriching target nucleic acid molecules from a nucleic acid containing sample which comprises target nucleic acid molecules and non-target nucleic acid molecules, wherein the target nucleic acid molecules are longer than the non-target nucleic acid molecules, the method comprising
(a) preparing a binding mixture comprising
   the nucleic acid containing sample,
   a poly(alkylene oxide) polymer and
   a salt
   and binding nucleic acid molecules to a solid phase which comprises a functional group, preferably carboxylated magnetic particles, wherein the bound nucleic acid molecules comprise target nucleic acid molecules;
(b) preferably separating the solid phase with the bound nucleic acid molecules from the remaining sample;
(c) contacting the solid phase with the bound nucleic acid molecules at least once with a reagent composition comprising a poly(alkylene oxide) polymer and a salt to selectively elute non-target nucleic acid molecules, wherein preferably, the concentration (w/v) of the poly(alkylene oxide) polymer in the reagent composition of step (c) is lower than the concentration (w/v) of the poly(alkylene oxide) polymer in the binding mixture of step (a);
(d) optionally washing the bound target nucleic acid molecules; and
(e) eluting the bound target nucleic acid molecules from the solid phase.

In one embodiment thereof, a poly(alkylene oxide) polymer based size selective nucleic acid enrichment method is provided for enriching target nucleic acid molecules having a size above a certain cut-off value from a nucleic acid containing sample, said method comprising
(a) preparing a binding mixture comprising
   the nucleic acid containing sample,
   a poly(alkylene oxide) polymer, and
   a salt
   and binding target nucleic acid molecules to a solid phase which comprises a functional group, preferably carboxylated magnetic particles, thereby providing a solid phase having bound thereto nucleic acid molecules having a size above the cut-off value, wherein under the used binding conditions non-target nucleic acid molecules having a size below the cut-off value do not bind to the solid phase or bind to a lesser extent to the solid phase compared to the longer target nucleic acid molecules;
(b) separating the solid phase with the bound nucleic acid molecules from the remaining sample;
(c) contacting the solid phase with the bound nucleic acid molecules at least once with a reagent composition comprising a poly(alkylene oxide) polymer and a salt to selectively elute non-target nucleic acid molecules having a size below the cut-off, wherein preferably, the concentration (w/v) of the poly(alkylene oxide) polymer in the reagent composition of step (c) is lower than the concentration (w/v) of the poly(alkylene oxide) polymer in the binding mixture of step (a);
(d) optionally washing the bound target nucleic acid molecules; and
(e) eluting the bound target nucleic acid molecules from the solid phase.

The present invention is based on the established size selective nucleic acid isolation technology, wherein at least one poly(alkylene oxide) polymer, such as a polyethylene glycol, is used in order to precipitate target nucleic acid molecules (which have a size above a certain cut-off value) from a nucleic acid containing sample. Precipitated target nucleic acid molecules are/become bound to the solid phase (e.g. providing a carboxylated surface), thereby providing a solid phase having bound thereto target nucleic acid molecules. Step (a) may essentially correspond to the prior art. The cut-off value for the nucleic acid molecules that are bound to the solid phase in step (a) can be adjusted by modifying the concentration of the poly(alkylene oxide) polymer in the binding mixture. In embodiments, target and non-target nucleic acids are bound to the solid phase in step (a). In preferred embodiments, step (a) is performed in a size-selective manner to preferentially bind to the solid phase target nucleic acid molecules wherein the smaller non-target nucleic acid molecules having a length below the cut-off value do not bind to the solid phase under the used binding conditions or bind to a lesser extent to the solid phase compared to the longer target nucleic acid molecules. If using size selective binding conditions in step (a), non-target nucleic acid molecules having a size below the cut-off value substantially do not bind to the solid phase under the used binding conditions or at least bind to a lesser extent to the solid phase compared to the longer target nucleic acid molecules. However, as discussed herein and also shown in the examples, even if size selective binding conditions are used in step (a), some residual non-target nucleic acid molecules that have a size below the cut-off value may nevertheless bind to the solid phase in step (a) and accordingly, are also separated together with the solid phase in step (b). This problem is known in the art. Such binding of non-target nucleic acids can in particular occur if the amount of smaller non-target nucleic acids in the nucleic acid containing sample is high, as it is e.g. the case in adapter ligated DNA samples provided during preparation of a sequencing library, which said samples usually comprising large amounts of small non-target DNA fragments such as adapter monomers and dimers. As is demonstrated in the examples, binding of non-target nucleic acids may also occur, if a high polymer concentration is used in step (a). As is described herein, non-target nucleic acid molecules that were bound in step (a) to the solid phase may be removed from the solid phase during step (c) by size selective elution. Step (c) allows to efficiently remove small non-target nucleic acid molecules that were bound as contaminants in step (a) by performing a size-selective elution step.

The target nucleic acid may be selected from DNA and RNA, and preferably is DNA. Therefore, according to a preferred embodiment the nucleic acid containing sample is a DNA containing sample and the target nucleic acid molecules are DNA molecules. The method is particularly suitable for enriching DNA fragments of a certain size during preparation of a sequencing library suitable for next generation sequencing, in particular for purifying adapter ligated DNA molecules (target nucleic acids) from adapter monomers and adapter-adapter ligation products (non-target nucleic acids). All disclosures and embodiments described in this application for a nucleic acid containing sample or nucleic acid molecules in general, specifically apply and particularly refer to these preferred embodiments wherein the target nucleic acid is DNA and accordingly, wherein the nucleic acid containing sample is a DNA containing sample, and in particular refer to the embodiment wherein the DNA containing sample is an adapter ligation sample which comprises adapter ligated DNA molecules (target nucleic acids) as well as adapter monomers and adapter-adapter ligation products (non-target nucleic acids). The individual method steps and preferred embodiments will now be described in detail.

### Step (a)

Step (a) comprises preparing a binding mixture which comprises
the nucleic acid containing sample,
a poly(alkylene oxide) polymer and
a salt.

The binding mixture comprises at least one poly(alkylene oxide) polymer. The contained poly(alkylene oxide) polymer, preferably a polyethylene glycol, precipitates target nucleic acid (preferably DNA) molecules so that they bind to the solid phase that is comprised in the binding mixture. Hence, step (a) comprises binding nucleic acid molecules to the solid phase, wherein the bound nucleic acid molecules comprise target nucleic acid molecules. Thereby, a solid phase is provided having bound thereto target nucleic acid molecules. As is described herein, the solid phase comprises a functional group, which is preferably acidic. Particularly suitable is a carboxyl group as functional group. In an advantageous embodiment, the solid phase is provided by carboxylated magnetic particles.

Step (a) is preferably performed in a size selective manner to deplete non-target nucleic acids already during the binding step while efficiently capturing the target nucleic acids. Hence, preferably target nucleic acid molecules having a size above a cut-off value bind to the solid phase, while under the used binding conditions non-target nucleic acid molecules having a length below the cut-off value do not bind to the solid phase or bind to a lesser extent to the solid phase compared to the longer target nucleic acid molecules. Under such binding conditions, non-target nucleic acid molecules having a molecular size which is less than the cut-off value substantially do not bind to the solid phase which provides e.g. a carboxylated surface (e.g. carboxylated magnetic particles). Thus, in step (a), target nucleic acid molecules having a size above the cut-off value efficiently bind to the solid phase while smaller non-target nucleic acid molecules having a size below the cut-off value preferably substantially do not bind to the solid phase in this size selective binding step (a) and thus remain in the binding mixture. Accordingly, after binding step (a), preferably a solid phase is provided (e.g. carboxylated magnetic particles) with bound nucleic acids, wherein the bound nucleic acids comprise the target nucleic acids, or preferably comprise substantially or consist essentially of the target nucleic acids. As is described herein, the cut-off value can be adjusted e.g. by varying the concentration of the at least one poly(alkylene oxide) polymer in the binding mixture.

According to a preferred embodiment, step (a) comprises adding a binding reagent to the nucleic acid containing sample to prepare the binding mixture, wherein the binding reagent comprises the poly(alkylene oxide) polymer, preferably a polyethylene glycol, and the salt. The binding reagent that is used in the present method is also referred to herein as “precipitation reagent”. Details of the binding reagent are described below. Preferably, the binding conditions are exclusively established by the binding reagent and no further additives or reagents are added to establish the binding conditions for binding the nucleic acid molecules having a size above a certain cut-off value to the solid phase which is also contacted with the binding mixture. This simplifies handling.

When using size-selective binding conditions in step (a), the average length of the target nucleic acid molecules that under the chosen binding conditions bind to the solid phase lies above the cut-off value while the average length of the non-target nucleic acid molecules which are not bound to the solid phase lies below the cut-off value. The expression that “target nucleic acid molecules having a size above the cut-off value bind to the solid phase” and similar expressions used herein, in particular specify that nucleic acid molecules having a size at the cut-off value or above bind to the solid phase. The terms “size” and “length” are used interchangeably herein. I.e. if the cut-off value for DNA molecules is described as being 200 nt, this means that target DNA molecules having a size of 200 nt or longer bind to the solid phase. Thus, the cut-off value in particular defines the size of the smaller non-target DNA molecules that substantially do not bind under the respective binding conditions to the solid phase. According to one embodiment, the cut-off value corresponds to the point where the curve of an electropherogram for the target nucleic acid, e.g. adapter ligated DNA library molecules representing the library, meets the x-axis. However, at this point, respectively this cut-off value, there is not necessarily a quantitative recovery of the DNA but the percentage of captured DNA molecules increases with increasing size of the DNA molecules.

The term “poly(alkylene oxide) polymer” as used herein in particular refers to an oligomer or polymer of alkylene oxide units. Poly(alkylene oxide) polymers are known in low and high molecular weights. The molecular weight is usually a multitude of the molecular weight of its monomer(s) (e.g. 44 in case of ethylene oxide), and can range up to e.g. 50000. The molecular weight is indicated in Da. The poly(alkylene oxide) polymer may be linear or branched. A linear poly(alkylene oxide) polymer is preferred. The poly(alkylene oxide) polymer may be unsubstituted or substituted. Substituted poly(alkylene oxide) polymers, include e.g. alkylpoly(alkylene oxide) polymers, e.g. alkylpolyethylene glycols, but also poly(alkylene oxide) esters, poly(alkylene oxide) amines, poly(alkylene oxide) thiol compounds and others. The alkylene oxide unit may be selected from the group consisting of ethylene oxide and propylene oxide. Also co-polymers such as e.g. of ethylene oxide and propylene oxide are encompassed by the term a poly(alkylene oxide) polymer. Preferably, the poly(alkylene oxide) polymer is a poly(ethylene oxide) polymer or a poly(propylene oxide) polymer, more preferably it is a polyethylene glycol or a polypropylene glycol. Polyethylene glycol is particularly preferred because it is also commonly used in size selective DNA isolation methods as is also evidenced by the prior art discussed above. However, also other poly(ethylene oxide) polymers may be used such as substituted poly(ethylene oxide) polymers, e.g. alkyl poly(ethylene oxide) polymers such as alkylpolyethylene glycols. Polyethylene glycol is preferably unbranched and may be unsubstituted or substituted. Known substituted forms of polyethylene glycol include alkylpolyethylene glycols that are e.g. substituted at one or both ends with a C1-C5 alkyl group.

Preferably, unsubstituted polyethylene glycol is used as poly(alkylene oxide) polymer in the present invention. Such unsubstituted polyethylene glycol has the formula HO—(CH₂CH₂O)ₙ—H, wherein n depends on the molecular weight. All disclosures described in this application for the poly(alkylene oxide) polymer in general specifically apply and particularly refer to the preferred embodiment polyethylene glycol, in particular unsubstituted polyethylene glycol, even if not explicitly stated.

The poly(alkylene oxide) polymer can be used in various molecular weights as is demonstrated by the examples. According to one embodiment, the poly(alkylene oxide) polymer, which preferably is a polyethylene glycol, has a molecular weight that lies in a range of 2000 to 40000. The poly(alkylene oxide) polymer may have a molecular weight that lies in a range of 2500 to 35000 or 3000 to 30000, such as 4000 to 25000 or 5000 to 20000. As is supported by the examples, particular suitable ranges include 3000 to 25000, such as 6000 to 25000 and 8000 to 20000. Preferred is a molecular weight in the range of 6000 to 20000, such as in the range of 6000 to 16000, such as 8000. Such molecular weights are particularly suitable for polyethylene glycol. Polyethylene glycol 3000, 8000 and 20000 was also used in the examples. As disclosed herein, the molecular weight of the poly(alkylene oxide) polymer is indicated in Da.

The poly(alkylene oxide) polymer is preferably present in the binding mixture in a concentration sufficient to precipitate at least target nucleic acid molecules, such as target DNA molecules, having a size above the cut-off value which then bind to the solid phase. As disclosed herein, the binding is preferably size-selective in that the smaller non-target nucleic acid does not bind or at least binds to a lesser extent, whereby non-target nucleic acids are already depleted during the initial binding step. The cut-off value is influenced and can be adjusted by the concentration of the poly(alkylene oxide) polymer in the binding mixture. Therefore, by varying the concentration of the poly(alkylene oxide) polymer in the binding mixture one may relocate and thus adjust the cut-off value. This allows to adjust the binding conditions so that target nucleic acids bind more efficiently to the solid phase than the smaller non-target nucleic acids. As is demonstrated in the examples, increasing the concentration of the poly(alkylene oxide) polymer in the binding mixture lowers the cut-off value. Such variation in the concentration in the binding mixture can e.g. be achieved by preparing different binding reagents each comprising the poly(alkylene oxide) polymer in a different concentration or by adding a different volume of the same binding reagent to the nucleic acid, preferably DNA, containing sample.

According to one embodiment, the poly(alkylene oxide) polymer concentration in the binding mixture is at least 5% (w/v). The concentration may be at least 6%, at least 7%, at least 8%, at least 9% or at least 10% (w/v). The binding mixture may comprise the poly(alkylene oxide) polymer, which preferably is a polyethylene glycol, in a concentration that lies in a range of 5% to 30% (w/v). The concentration may e.g. lie in a range selected from 6% to 25%, 7% to 20% and 7.5% to 15% (w/v). Particularly suitable are concentrations in a range of 8% to 14% and 8.5% to 13% (w/v). All % with respect to the polymer are indicated as (w/v). The indicated concentrations are particularly suitable for polyethylene glycol. As disclosed herein, the concentration may be chosen such that binding occurs in a size-selective manner to preferentially bind the target nucleic acids to the solid phase, while depleting non-target nucleic acids already during the binding step. In order to ensure efficient precipitation and binding of the target nucleic acid molecules having a size above the cut-off value to the solid phase, the binding mixture may comprise the poly(alkylene oxide) polymer in a concentration of at least 7.5% (w/v), such as in a range of 8% to 13% or 8.5% to 12.5% for polyethylene glycol. The required concentration depends on the length of the target nucleic acid molecules and can be adapted based on the teachings provided herein.

The binding mixture comprises at least one salt. The salt promotes binding of the target nucleic acid molecules having a size above the intended cut-off value to the solid phase. The salt can be a monovalent salt. As is demonstrated in the examples, a non-chaotropic salt is preferably used as salt. The salt may be an alkali metal salt, preferably a halide such as a chloride salt. It may be selected from sodium chloride, potassium chloride, lithium chloride and cesium chloride, e.g. selected from sodium chloride and potassium chloride. In one embodiment, the salt is sodium chloride. The use of a non-chaotropic alkali metal salt is preferred. According to one embodiment, the binding mixture does not comprise a chaotropic salt such as guanidinium salts, iodides, thiocyanates or perchlorates. Preferably, the binding mixture does not comprise other chaotropic salts of equal or stronger chaotropic nature either. In embodiments, the binding mixture does not comprise a C1-C8 alkanol in addition to the poly(alkylene oxide) polymer.

Suitable concentrations for the salt in the binding mixture are known from the prior art and can be determined by the skilled person based on the teachings provided herein. The binding mixture may comprise the salt in a concentration of ≥350 mM. The concentration may be ≥500 mM, ≥700 mM or ≥800 mM. Particularly suitable is a salt concentration of ≥900 mM and ≥1M. The salt may be present in the binding mixture in a concentration that lies in a range of 350 mM to 3.5M. Exemplary ranges include e.g. 500 mM to 3M, 700 mM to 2.5M and 800 mM to 2.25M. Particularly suitable is a salt concentration in the binding mixture that lies in a range of 900 mM to 2M, preferably 1M to 1.75M. The salt is preferably a monovalent salt, in particular an alkali metal salt such as NaCl or KCl.

As disclosed herein, step (a) preferably comprises adding a binding reagent to the nucleic acid containing sample to prepare the binding mixture, wherein the binding reagent comprises the poly(alkylene oxide) polymer, preferably a polyethylene glycol, and the salt. The binding reagent is preferably liquid. It may be provided in form of a solution (which may comprise the solid phase, e.g. magnetic particles). A binding reagent that is added to the nucleic acid containing sample to prepare the binding mixture comprises the poly(alkylene oxide) polymer (preferably polyethylene glycol) and the salt in an amount that achieves the desired concentration in the binding mixture when contacting the intended volume of the nucleic acid containing sample with an appropriate volume of the binding reagent.

Suitable embodiments for the poly(alkylene oxide) polymer have been described above. The binding reagent may comprise the poly(alkylene oxide) polymer, which preferably is a polyethylene glycol, in a concentration of 7% to 50% (w/v). Suitable concentrations in the binding reagent may lie e.g. in a range selected from 10% to 45%, such as 12% to 40% and 15% to 35%. All % with respect to the polymer are indicated as (w/v). A molecular weight that lies in a range of 3000 to 30000, e.g. selected from 4000 to 25000, 5000 to 25000, 6000 to 20000 and 6000 to 16000, e.g. 8000, is particularly suitable. A volume of the binding reagent may be mixed with the nucleic acid containing sample to prepare a binding mixture that comprises the polymer, preferably a polyethylene glycol, in the above described concentrations. It is referred to the above disclosure.

Suitable embodiments for the salt have been described above. The binding reagent may comprise the salt, which preferably is an alkali metal salt, in a concentration that lies in the range of 0.5M to 5M. Suitable concentration ranges include but are not limited to 0.7M to 4.5M, 1M to 4.25M and 1.25M to 4M. Particularly preferred concentration ranges for the salt in the binding reagent are 1.5M to 3.75M and 1.75M to 3.5M. A volume of the binding reagent may be mixed with the nucleic acid containing sample to prepare a binding mixture that comprises the salt in the above described concentrations. It is referred to the above disclosure. As disclosed herein, it is preferred that the salt is a non-chaotropic salt.

According to one embodiment, a binding reagent is added in step (a) to prepare the binding mixture wherein the binding reagent comprises
a polyethylene glycol having a molecular weight that lies in the range of 3000 to 25000, preferably 4000 (or 5000) to 25000, such as 6000 to 20000 or 6000 to 16000; and
an alkali metal salt in a concentration that lies in the range 0.5M to 5M, preferably 0.7M to 4.5M, more preferably 1M to 4.25M, more preferably 1.5M to 3.75M or 1.75M to 3.5M.

According to one embodiment, a binding reagent is added in step (a) to prepare the binding mixture wherein the binding reagent comprises
a polyethylene glycol having a molecular weight that lies in a range of 5000 to 25000, e.g. selected from 6000 to 20000 and 6000 to 16000, such as 6000 to 10000; and
an alkali metal salt in a concentration that lies in the range of 1M to 4M, preferably 1.5M to 3.75M, more preferably 2M to 3.5M.

According to one embodiment, a binding reagent is added in step (a) to prepare the binding mixture wherein the binding reagent comprises
a polyethylene glycol having a molecular weight that lies in a range of 5000 to 25000, such as 6000 to 20000, in a concentration that lies in a range of 7% to 40% (w/v), e.g. selected from 10% to 40%, 12% to 35% and 15% to 30%; and
an alkali metal salt in a concentration that lies in a range of 1M to 4M, e.g. selected from 1.5M to 3.75M and 2M to 3.5M.

According to one embodiment, a binding reagent is added in step (a) to prepare the binding mixture wherein the binding reagent comprises
a polyethylene glycol having a molecular weight that lies in a range of 5000 to 25000, such as 6000 to 20000 or 6000 to 16000, in a concentration that lies in a range of 10% to 35%, preferably 15% to 30%;
an alkali metal salt, preferably selected from sodium chloride and potassium chloride, in a concentration selected from 1.5M to 3.5M and 2M to 3M.

Further binding reagents are also disclosed in the claims.

The binding reagent may comprise additional components. Exemplary components include but are not limited to a surfactant (e.g. a non-ionic surfactant) or a chelating agent. Chelating agents include, but are not limited to diethylenetriaminepentaacetic acid (DTPA), ethylenedinitrilotetraacetic acid (EDTA), ethylene glycol tetraacetic acid (EGTA) and N,N-bis(carboxymethyl)glycine (NTA) and furthermore, e.g. citrate or oxalate. EDTA is preferred. The binding reagent may also comprise a buffering agent.

According to one embodiment, the binding reagent does not comprise a chaotropic salt such as guanidinium salts, iodides, thiocyanates, perchlorates or other chaotropic salts of equal or stronger chaotropic nature. In embodiments, the binding reagent does not comprise a C1-C8 alkanol.

According to one embodiment, the binding reagent comprises the solid phase, which preferably is provided by particles, more preferably by magnetic particles. The magnetic particles, such as carboxylated magnetic particles, may be suspended in the liquid binding reagent. The binding reagent comprising the solid phase may then be contacted with the nucleic acid containing sample to prepare the binding mixture.

The binding conditions (and hence the cut-off value) can be and preferably are controlled/adjusted by the binding reagent that is added to the DNA containing sample.

According to one embodiment, step (a) comprises adding X volume binding reagent to 1 volume nucleic acid containing sample, wherein X is a number that is selected from (i) 0.25 to 2, such as (ii) 0.5 to 1.75, (iii) 0.6 to 1.6, (iv) 0.7 to 1.5, preferably (v) 0.8 to 1.4 and more preferably (vi) 0.9 to 1.3.

Contacting the binding reagent with the nucleic acid containing sample reduces the concentration of the ingredients contained in the binding reagent in the resulting binding mixture due to a dilution effect of the nucleic acid containing sample, which preferably is a liquid sample. By adding e.g. different amounts of the liquid binding reagent to the DNA containing sample, one may flexibly adjust the concentration of the polymer (preferably PEG) in the binding mixture and thus relocate the cut-off value.

As is described above, the binding mixture is preferably prepared by contacting the sample, which preferably is a DNA containing sample with a binding reagent. In one embodiment, the binding reagent may have a pH value that lies in a range of 4 to 10. A suitable pH may lie e.g. in a range of 4.5 to 9.5 and 5 to 9. In one embodiment, the pH lies in a range of 7 to 8.5. Such pH is particularly suitable when using a solid phase comprising carboxyl groups. When using a solid phase comprising carboxyl groups, such as carboxylated magnetic particles, which is preferred, the pH can vary over a broad range as is demonstrated in the examples. To maintain the pH, the binding reagent may comprise a buffering agent.

According to one embodiment, the binding conditions such as the concentration of the poly(alkylene oxide) polymer and the salt are established by contacting the nucleic acid containing sample with the binding reagent. Preferably, no further adjustments are made to establish the binding conditions in the binding mixture. Thus, preferably, the binding mixture is provided exclusively by contacting the binding reagent with the nucleic acid containing sample and the solid phase but no further buffers or other reagents are added to establish the binding conditions for binding the precipitated target nucleic acid molecules to the solid phase. This advantageously avoids handling and adjustment errors. Furthermore, as is described herein, the binding reagent may also advantageously be used to prepare the reagent composition of step (c) by adding a dilution solution.

The nucleic acid containing sample may comprise DNA and/or RNA. Poly(alkylene oxide) polymer based size selective nucleic acid enrichment is known for DNA and RNA. DNA is a more common application and preferred. Preferably, the nucleic acid containing sample is thus a DNA containing sample. The nucleic acids in the DNA containing sample comprise or consist of DNA molecules of different sizes (lengths). The DNA containing sample may comprise single-stranded and/or double stranded DNA. Preferably, the DNA molecules are linear, double-stranded DNA molecules. The DNA containing sample can be of various origins, including but not limited to biological samples and artificial samples that were obtained during nucleic acid processing. The DNA containing sample is preferably a liquid sample. According to one embodiment, the DNA containing sample is not a lysate. According to one embodiment, the DNA containing sample is a sample of extracted DNA or extracted DNA that has been further processed, e.g. by shearing or by way of an enzymatic reaction. According to one embodiment, the DNA containing sample was obtained after an enzymatic reaction. Exemplary enzymatic reactions that provide DNA containing samples that can be processed using the size selective DNA enrichment method of the invention include but are not limited to amplification reactions, ligase reactions, in particular adapter ligation reactions and polynucleotide, e.g. poly A, tailing reactions. According to one embodiment, the DNA containing sample comprises fragmented DNA, such as e.g. sheared DNA. According to one embodiment, the DNA containing sample comprises sheared genomic DNA or sheared cDNA. According to one embodiment the DNA containing sample is a composition resulting from a size shearing procedure. Such DNA containing sample comprises DNA fragments of different sizes. Said fragmented DNA may also be end-repaired to provide DNA fragments having blunt ends. Thus, according to one embodiment, the DNA containing sample comprises linear, blunt-ended DNA fragments of different sizes. According to a preferred embodiment, the DNA containing sample was obtained during the preparation of a sequencing library, in particular during preparation of a next generation sequencing library. According to one embodiment, the DNA containing sample is an adapter ligation sample that was obtained as a result of an adapter ligation step, e.g. during preparation of a sequencing library. According to a preferred embodiment, the DNA containing sample is an adapter ligation sample which comprises (i) double-stranded DNA molecules that are flanked 5′ and/or 3′ by adapters, (ii) adapter monomers and (iii) adapter-adapter ligation products such as e.g. adapter dimers. Furthermore, the DNA containing sample may comprise additional contaminating components such as e.g. mono, oligo- and/or polynucleotides and proteins such as enzymes that are e.g. still present in the DNA containing sample from previous enzymatic sequencing library processing steps. The method according to the present invention allows inter alia to size selectively purify double-stranded DNA molecules that are flanked 5′ and/or 3′ by adapters, preferably are flanked at their 5′ end and their 3′ end by adapters, thereby efficiently removing respective contaminants. According to one embodiment, the DNA containing sample comprises amplification products, e.g. PCR products. Thus, according to one embodiment, the DNA containing sample is a sample resulting from an amplification procedure, in particular resulting from a PCR amplification.

The binding conditions and in particular the concentration of the poly(alkylene oxide) polymer in the binding mixture may be chosen such that a desired cut-off value is obtained that allows e.g. to remove undesired small DNA molecules such as e.g. adapters, adapter-adapter ligation products or primers during the binding step, while effectively binding target DNA molecules having a size above the cut-off value to the solid phase. The precise cut-off value to be chosen depends e.g. on the intended use of the target DNA molecules and also the size of e.g. contaminating small DNA molecules that are not supposed to be bound and thus are supposed to be removed during size selection. The cut-off value can thus be chosen by the skilled person in accordance with the intended application of the purified target DNA molecules.

The sizes, respectively cut-off values indicated herein with reference to nucleotides “nt”, refer to the chain length of the nucleic acid molecules, which preferably are DNA molecules, and thus are used in order to describe the length of, respectively describe the cut-off value for single-stranded as well as double-stranded nucleic acid molecules. In double-stranded DNA molecules said nucleotides are paired. Hence, if the DNA is a double stranded molecule, what is preferred, the indications with respect to the size or length in “nt” refers to “bp”. Thus, if a double-stranded DNA molecule has a chain length, respectively size, of 100 nt, said double-stranded DNA molecule has a size of 100 bp. The same applies to the definition of the cut-off value for double-stranded DNA molecules.

According to one embodiment, a size-selective binding step is performed in step (a) so that predominantly target nucleic acid molecules having a size above a chosen cut-of value bind to the solid phase, while non-target nucleic acids having a size (length) below the cut-off value do not bind to the solid phase or bind to a lesser extent compared to the longer target nucleic acids. The cut-off value is chosen so that it at least allows to remove non-target nucleic acid molecules having a size of 150 nt or less, such as 140 nt or less. In embodiments, the cut-off value is chosen so that it allows to remove non-target nucleic acid molecules having a size of 180 nt or less or 200 nt or less. Such embodiments are particularly suitable for size selection during the preparation of a sequencing library. Here, a size selective DNA isolation step is in particular performed in order to separate adapter ligated DNA molecules from unligated adapter monomers and adapter-adapter ligation products, such as adapter dimers. The size of adapters that are commonly used for preparing sequencing libraries for next generation sequencing often lies in the range of 25 nt to 75 nt, in particular 30 nt to 60 nt. For removing unligated adapter monomers and adapter-adapter ligation products (such as in particular adapter dimers), the cut-off value is according to one embodiment chosen such that it lies above the size of the adapter monomer(s) and above the size of the expected adapter-adapter ligation product(s). Preferably, the cut-off value is at least 20 nt, at least 25 nt or at least 30 nt larger than the expected size of adapter-adapter ligation product(s) in order to ensure an efficient removal of the adapter monomer(s) and adapter-adapter ligation product(s). For removing adapter monomers and adapter-adapter ligation products, the cut-off value may be 150 nt or less or 140 nt or less. According to one embodiment, the target nucleic acid molecules comprised in the nucleic acid containing sample have a size in a range of 200 nt to 3000 nt, such as 250 nt to 2500 nt or 300 nt to 2000 nt. An according size range is e.g. common for DNA fragments (such as adapter ligated DNA molecules) that are found in DNA libraries, in particular DNA libraries prepared for sequencing. The DNA fragments comprised in a sequencing library often have a size distribution that is encompassed by the above ranges. E.g. in the present examples, the target DNA fragments (comprised in a sequencing library) have in embodiments a size lying somewhere in a range of 200 nt to 1800 nt (bp) as is evident from the indicated size distributions. The non-target nucleic acid molecules (adapter monomers and adapter dimers) that were additionally comprised as contamination in the starting material were smaller in size, having predominantly a size up to 130 nt (bp). In the method of the invention the cut-off is chosen so that non-target nucleic acid molecules are efficiently depleted during the performed size selection steps, while target nucleic acid molecules are bound to the solid phase and become purified from the non-target nucleic acids. As is also known in the art and evident from the present examples, DNA libraries may differ in their average fragment size. According to one embodiment, the nucleic acid containing sample comprises a DNA library to be purified, wherein the target DNA molecules of said library have an average fragment size that lies in a range selected from 300 nt to 1000 nt, 300 nt to 700 nt or 300 nt to 500 nt. In a further embodiment, the target DNA molecules of the DNA library have an average fragment size that lies in a range of 500 nt to 1000 nt or 700 nt to 1500 nt. The average fragment length can be determined e.g. by using e.g. a Bioanalyzer. In embodiments, the cut-off is chosen such that non-target DNA molecules (adapter monomers and adapter-adapter ligation products) having a size of 150 nt or less or 40 nt or less can be depleted, while enriching the target nucleic acids.

Suitable solid phases can embody a variety of shapes and include, but are not limited to, particles, fibers, filter, a membrane or other supports on which a precipitated nucleic acid can bind. Suitable solid phases have sufficient surface area to permit efficient binding of nucleic acids. The use of particles, in particular magnetic particles, as solid phase is preferred.

A variety of surfaces may be utilized as is known in the prior art. The solid phase may comprise a surface which is coated with functional moieties which reversibly bind the nucleic acid under the used binding conditions. The functional group may act as a bioaffinity adsorbent for polyalkylene glycol precipitated nucleic acid such as DNA. Suitable functionalized solid phases that can be used in order to bind precipitated nucleic acids in poly(alkylene oxide) polymer based size selective nucleic acid isolation methods are well-known in the art and therefore, do not need any detailed description. The functional groups may be of the same or different type and may be provided by ionic groups, e.g. ion exchange groups, preferably acidic groups. Acidic groups can be provided by carboxyl groups, sulfonate groups and silane ligands. Preferably, the solid phase comprises carboxyl groups. In one embodiment, the solid phase includes a surface coating that provides the functional groups such as carboxyl groups.

In a preferred embodiment, the solid phase provides a surface comprising carboxyl groups, also referred to herein as carboxylated surface. As is described herein and demonstrated in the examples, the use of a solid phase comprising carboxyl groups at the surface is particularly suitable and preferred in the context of the present invention. Unless indicated otherwise, all disclosures and embodiments described in this application for the use of a solid phase in general, specifically apply and particularly refer to this preferred embodiment wherein the solid phase comprises carboxyl groups, more preferably wherein the solid phase is provided by carboxylated magnetic particles.

In general, and by way of example, a carboxylated surface is a surface that is coated with or encompasses one or more carboxyl groups or moieties that are capable of reversibly and non-specifically associating with nucleic acid. Methods for coating a solid phase with functional groups, either directly or indirectly, are known in the art. For example, the functional groups (e.g. the carboxyl group COOH) can coat a solid phase during formation of the solid phase. In addition, the solid phases can be coated with functional groups by covalently coupling a functional group (one or more) to a COOH group (one or more) on the solid phase. A suitable moiety with a free carboxylic acid functional group is a succinic acid moiety in which one of the carboxylic acid groups is bonded to the amine of amino silanes through an amide bond and the second carboxylic acid is unbonded, resulting in a free carboxylic acid group attached or tethered to the surface of the solid phase.

According to one embodiment, particles are used as solid phase that may have the form of beads. The particles may have a size of about 0.02 to 25 μm, such as 0.1 to 15 μm, 0.125 to 12.5 μm, 0.15 to 10 μm and 0.2 to 7 μm. To ease the processing of the nucleic acid binding solid phase, preferably magnetic particles are used. Magnetic particles respond to a magnetic field. The magnetic particles may e.g. be ferrimagnetic, ferromagnetic, paramagnetic or superparamagnetic. Paramagnetic particles are particularly preferred. Paramagnetic particles can be efficiently separated from a solution using a magnet, but can be easily resuspended without magnetically induced aggregation occurring. Paramagnetic particles may comprise a magnetite rich core encapsulated by a polymer shell.

According to a preferred embodiment the solid phase is provided by carboxylated magnetic particles, wherein preferably, the magnetic particles are paramagnetic. Carboxylated magnetic particles are also commercially available, and include but are not limited to Sera-Mag Speed Beads (Sigma Aldrich, GE), Agencourt AMPure XP, QIAseq, M-Beads (MoBiTec).

The use of magnetic particles has advantages, because the magnetic particles including the bound nucleic acids can be processed easily by the aid of a magnetic field, e.g. by using a permanent magnet. This embodiment is e.g. compatible with established robotic systems capable of processing magnetic particles. Different robotic systems exist in the prior art that can be used in conjunction with the present invention to process the magnetic particles to which the target DNA molecules were bound. According to one embodiment, magnetic particles are collected at the bottom or the side of a reaction vessel and the remaining liquid sample is removed from the reaction vessel, leaving behind the collected magnetic particles to which the DNA molecules are bound. Removal of the remaining sample can occur by decantation or aspiration. Such systems are well known in the prior art and thus need no detailed description here. In an alternative system that is known for processing magnetic particles the magnet which is usually covered by a cover or envelope plunges into the reaction vessel to collect the magnetic particles. As respective systems are well-known in the prior art and are also commercially available (e.g. QIASYMPHONY®; QIAGEN), they do not need any detailed description here. In a further alternative system that is known for processing magnetic particles, the sample comprising the magnetic particles can be aspirated into a pipette tip and the magnetic particles can be collected in the pipette tip by applying a magnet e.g. to the side of the pipette tip. The remaining sample can then be released from the pipette tip while the collected magnet particles which carry the bound target DNA molecules remain due to the magnet in the pipette tip. The collected magnetic particles can then be processed further. Such systems are also well-known in the prior art and are also commercially available (e.g. BioRobot EZ1, QIAGEN) and thus, do not need any detailed description here.

According to one embodiment, the solid phase is comprised in a column. The term “column” as used herein in particular describes a container having at least two openings. Thereby, a solution and/or sample can pass through said column. The term “column” in particular does not imply any restrictions with respect to the shape of the container which can be e.g. round or angular and preferably is cylindrical. However, also other shapes can be used, in particular when using multi-columns. Said solid phase comprised in the column should allow the passage of a solution, respectively the binding mixture when applied to the column. This means that if e.g. a centrifuge force is applied to the column, a solution and/or the binding mixture is enabled to pass through the column in direction of the centrifuge force. When using a column based isolation procedure, the binding mixture is usually passed through the column, e.g. assisted by centrifugation or vacuum, and the nucleic acid molecules having a size above the cut-off value bind to the comprised solid phase during said passage.

Contacting the nucleic acid containing sample with the binding reagent to provide the binding mixture and binding of the target nucleic acid molecules having a size above a cut-off value to the solid phase may be performed simultaneously or sequentially. According to one embodiment, the nucleic acid containing sample is contacted with the binding reagent and the resulting binding mixture is then contacted with the solid phase. When using a particulate solid phase, the solid phase, the binding reagent and the nucleic acid containing sample can be added in any order. E.g. it is within the scope of the present invention to first provide the solid phase and the binding reagent (e.g. in form of a suspension) and then add the sample or to first provide the sample, the solid phase and then add the binding reagent. Preferably, the binding reagent is mixed with the nucleic acid containing sample to provide the binding mixture.

As discussed herein, the solid phase comprising a functional group is preferably provided by particles, more preferred magnetic particles, such as carboxylated magnetic particles. The particles may be comprised in the binding reagent. Binding can be supported by agitation, e.g. incubation on a shaker or other agitating instrument.

At the end of step (a), nucleic acid molecules are bound to the solid phase, wherein the bound nucleic acids comprise, substantially comprise or consist essentially of the target nucleic acid molecules.

### Step (b)

Separation step (b) is preferably performed in order to separate the bound target nucleic acids from the sample remainders. It allows e.g. to separate non-target nucleic acids that were not bound to the solid phase in step (a). Alternatively, one may also dilute the binding mixture to lower the concentration of the polymer in the binding mixture by adding a dilution solution to thereby to lower the concentration of the polymer (and the salt) in the binding mixture to thereby prepare the elution composition of step (c).

However, step (b) is preferably performed and the nucleic acid molecules that are bound to the solid phase are separated from the remaining sample. Thereby, the bound larger nucleic acid molecules having a size above the cut-off value are separated from unbound nucleic acid molecules present in the sample. Suitable separation methods are well known in the prior art and the appropriate separation technique also depends on the used solid phase. When using a particulate solid phase, which is preferred, the particles can be collected by sedimentation which can be assisted by centrifugation. Preferably, separation is performed with the aid of a magnet (magnetic separation) if magnetic particles are used what is preferred. E.g. the supernatant can be separated off (e.g. decanted or aspirated) or the particles with the bound nucleic acids can be taken out of the liquid binding mixture. Suitable embodiments were described above in conjunction with the different formats of the solid phase and are well-known to the skilled person and are also described above.

### Step (c)

Step (c) comprises contacting the solid phase with the bound nucleic acid molecules at least once with a reagent composition comprising a poly(alkylene oxide) polymer and a salt to selectively elute non-target nucleic acid molecules having a size below the cut-off from the solid phase. In step (c), the concentration (w/v) of the poly(alkylene oxide) polymer is preferably lower than the concentration (w/v) of the poly(alkylene oxide) polymer in the binding mixture of step (a).

As discussed above, even if size-selective binding conditions are used in binding step (a), which is preferred, some smaller non-target nucleic acids may nevertheless bind to the solid phase, in particular if the amount of such smaller non-target nucleic acids is high in the nucleic acid containing sample. These undesired non-target nucleic acids bound to the solid phase must therefore be efficiently removed in order to provide the target nucleic acids having a size above the cut-off value as pure as possible. In the present method, this is achieved by performing step (c) at least once after separation step (b).

The advantages of performing step (c) have been explained above. During this selective elution step, smaller non-target nucleic acids are eluted, while the longer target nucleic acids remain bound to the solid phase. As discussed, step (c) efficiently removes from the solid phase non-target nucleic acid molecules that are shorter than the target nucleic acids and have a size below the cut-off value, thereby significantly improving the efficiency of size-selection without the necessity to perform several “bind-wash-elute” cycles of size selection as it is required in prior art methods. Step (c) allows to achieve a size selective elution of non-target nucleic acids having a size below the cut-off value from the solid phase thereby further purifying bound target nucleic acids which are/remain bound to the solid phase during step (c). As is demonstrated in the examples, the removal of small non-target nucleic acid molecules that is achieved with the present invention is very efficient due to processing step (c), which significantly improves the removal of unwanted small nucleic acid molecules and thus the purity of the target nucleic acid molecules. Step (c) achieves that such non-target nucleic acid molecules having a size below the chosen cut-off value are efficiently depleted. The removal of non-target nucleic acid molecules that is achieved with the present invention does not necessarily have to be complete. For some applications it is sufficient that unwanted small non-target nucleic acid molecules are depleted to an extent so that they do not disturb or hamper the intended downstream reaction. As is demonstrated in the examples, there might be some loss of target nucleic acids in step (c) if stringent conditions are used in step (c) to efficiently deplete and thus remove the non-target nucleic acids from the solid phase to thereby achieve a high purity of the target nucleic acids. However, such loss can be avoided by a sufficiently high concentration of the poly(alkylene oxide) polymer in step (c). Suitable conditions can be chosen by the skilled person based on the disclosure provided herein. The depletion efficiency and thus purity of the target nucleic acid molecules can be adjusted by the stringency of the conditions in step (a) and step (c). As is demonstrated in the examples, a very high removal efficiency and purity can be achieved with the present method.

The solid phase with the bound nucleic acids is contacted with the reagent composition in step (c). Several embodiments are feasible, also depending on the type of solid phase used. When using a column, the reagent composition (c) may be added to the column so that it may flow through the column. As disclosed herein, the use of particles, in particular magnetic particles, such as carboxylated magnetic particles, is preferred. E.g. after separation, the solid phase may be transferred into the prepared reagent composition, respectively the reagent (c). Alternatively, the reagent composition, preferably in form of a single reagent, may be added to the solid phase. These embodiments are particularly feasible when using particles, such as magnetic particles, as solid phase. The reagent composition may also be prepared by contacting a first reagent comprising the poly(alkylene oxide) polymer and a second reagent comprising the salt, either sequentially (in any order) or simultaneously, with the particles providing the solid phase, to achieve contacting the solid phase with the bound nucleic acid molecules with the reagent composition that establishes the conditions of step (c). Preferably, the reagent composition is provided by a single reagent composition, also referred to herein as reagent (c), e.g. an aqueous solution or buffer, that comprises the poly(alkylene oxide) polymer and the salt. As disclosed herein, the reagent composition can be advantageously prepared from the binding reagent, e.g. by diluting the binding reagent with a suitable dilution reagent (e.g. a TE buffer as shown in the examples) in order to adjust the polymer concentration for the size-selective elution step (c).

The reagent composition that is used in step (c), which preferably is provided by a single liquid reagent composition, also referred to herein as reagent (c), may have one or more of the characteristics of the binding reagent described above. As disclose herein, it may be prepared from the binding reagent by dilution with a dilution reagent. Details regarding the type, molecular weight and concentration of the poly(alkylene oxide) polymer, which preferably is a polyethylene glycol, the type and concentration of the salt and potential further ingredients such as buffering agents, non-ionic detergents and chelating agents as well as components that are not comprised in embodiments were described above in conjunction with step (a) and it is referred to the above disclosure which also applies to the reagent composition/reagent used in step (c).

The reagent composition/reagent (c) that is used in step (c) for selectively eluting and thus removing small non-target nucleic acid molecules having a size below the cut-off value from the solid phase may comprise the poly(alkylene oxide) polymer, which preferably is a polyethylene glycol, in a concentration of at least 5% (w/v), such as at least 6%, at least 7%, at least 8%, at least 9% or at least 10% (w/v). According to one embodiment, the reagent composition/reagent (c) comprises the poly(alkylene oxide) polymer, which preferably is a polyethylene glycol, in a concentration that lies in a range of 5% to 20% (w/v). Suitable concentration ranges include but are not limited to 6% to 18%, 6.5% to 16% and 6.5% to 15% (w/v). Particularly suitable are concentrations in the range of 7% to 14 (w/v), such as 7% to 13%, 7.5% to 12% (w/v) and 8% to 12.5% (w/v). As is shown in the examples, particularly suitable is a range of 7% to 12%, such as 8% to 11% (w/v). As disclosed herein, the concentration (w/v) of the polymer in the reagent composition of step (c) is preferably lower compared to the concentration (w/v) used in the binding mixture. Suitable concentrations in the reagent composition (c) can be chosen depending on the desired cut-off for eluting non-target nucleic acids in step (c).

The molecular weight of the poly(alkylene oxide) polymer, which preferably is a polyethylene glycol, may lie in a range of 2000 to 40000, such as 3000 to 30000 or 4000 to 30000. As shown in the examples, the molecular weight of the polymer that is used in step (c) for size-selective elution strongly influences the result. Polymers of higher molecular weights achieved more robust results in that the target nucleic acids remained bound to the solid phase (e.g. particles) during the size selective elution, while the non-target nucleic acid could be efficiently eluted. Preferably, the molecular weight lies in a range of 4000 or 5000 to 25000, such as 6000 to 25000, 6000 to 20000 or 8000 to 20000. Another suitable range is 6000 to 16000, such as 6000 to 10000, e.g. 8000.

According to a preferred embodiment, polyethylene glycol is used as poly(alkylene oxide) polymer and the same type of polyethylene glycol is used in step (a) and step (c). The polyethylene glycol used in steps (a) and (c) may have the same molecular weight. According to a further embodiment, a polyethylene glycol of differing molecular weight is used in step (a) and step (c), wherein the molecular weight of the polymer that is used in step (c) is higher or lower than the molecular weight of the polyethylene glycol that is used in step (a). Preferably, the molecular weight of the polyethylene glycol that is used in step (c) is either the same or higher than the molecular weight of the polyethylene glycol that is used in step (a).

The reagent composition that is contacted with the solid phase in step (c), respectively reagent (c), may comprise the salt in a concentration of 350 mM. The concentration in the reagent composition, respectively reagent (c), may be 500 mM, ≥700 mM or ≥800 mM. Particularly suitable is a salt concentration of 900 mM and 1M. The salt may be comprised in a concentration that lies in a range of 350 mM to 3.5M. Exemplary ranges include e.g. 500 mM to 3M, 700 mM to 2.5M and 800 mM to 2.25M. Particularly suitable is a reagent composition, respectively reagent (c), that comprises the salt in a concentration that lies in a range of 900 mM to 2M or 1M to 1.75M. The salt is preferably a monovalent salt, more preferably an alkali metal salt such as NaCl or KCl. The salt is preferably a non-chaotropic salt. The salt and suitable salt concentrations were also described in detail above and it is referred to the above disclosure which also applies here. The reagent composition, respectively reagent (c), preferably does not comprise a chaotropic salt. It is referred to the disclosure of the binding reagent which also applies here.

According to one embodiment, the solid phase is contacted in step (c) with a reagent composition (c) which comprises
a polyethylene glycol having a molecular weight that lies in a range selected from 2000 to 40000, preferably in a range selected from 3000 to 35000, 4000 to 30000, 5000 to 25000, 6000 to 25000 and 6000 to 20000, such as 6000 to 16000; and
an alkali metal salt in a concentration that lies in a range of 350 mM to 3.5M, e.g. in a range selected from 500 mM to 3M, 700 mM to 2.5M, 800 mM to 2.25M, 900 mM to 2M and 1M to 1.75M. As disclosed herein, the salt is preferably a non-chaotropic salt such as NaCl or KCl.

Suitable and preferred molecular weights and concentrations for polyethylene glycol are also described above and can be used in said reagent (c). As disclosed herein, reagent composition (c) may be prepared by diluting the binding reagent with a dilution reagent (e.g. a TE buffer).

According to one embodiment, the solid phase is contacted in step (c) with a reagent composition (c) which comprises
a polyethylene glycol having a molecular weight that lies in a range of 3000 to 30000, preferably selected from 5000 to 25000, such as in a range of 6000 to 25000, e.g. 6000 to 20000, 6000 to 16000 or 8000 to 16000; and
an alkali metal salt in a concentration that lies in a range of 500 mM to 2.5M, preferably selected from 700 mM to 2.25M, 900 mM to 2M and 1M to 1.75M.

According to one embodiment, the solid phase is contacted in step (c) with a reagent composition (c) which comprises
a polyethylene glycol having a molecular weight that lies in a range of 5000 to 25000, such as in a range of 6000 to 25000, 6000 to 20000 or 8000 to 20000, in a concentration that lies in a range of 6% to 20% (w/v), e.g. selected from 6.5% to 15%, 7% to 13% and 7.5% to 12%; and
an alkali metal salt in a concentration of 500 mM to 2.5M, e.g. selected from 700 mM to 2.25M, 900 mM to 2M and 1M to 1.75M.

According to one embodiment, the solid phase is contacted in step (c) with a reagent composition (c) which comprises
a polyethylene glycol having a molecular weight that lies in a range of 5000 to 25000, such as in a range of 6000 to 25000, 6000 to 20000 or 8000 to 20000, in a concentration that lies in a range selected from 6.5% to 15% (w/v), such as 7% to 13%, 7.5% to 12% and 8% to 11.5% (w/v); and
an alkali metal salt in a concentration selected from 800 mM to 2M, 900 mM to 1.75M and 1M to 1.5M.

According to one embodiment, the solid phase with the bound nucleic acid is only contacted with a single reagent composition (c) in step (c), but not with further reagents, such as further solutions. Therefore, according to one embodiment, the selective elution/binding conditions used in step (c) are exclusively established by reagent (c). However, as is demonstrated in the examples, reagent (c) may be advantageously provided, respectively be freshly prepared, by mixing e.g. the binding reagent that is used in step (a) with a dilution solution or buffer in order to provide/prepare reagent (c) that is then contacted with the separated solid phase. However, different contacting orders are also feasible.

The concentration of the poly(alkylene oxide) polymer and/or the concentration of the salt in the reagent composition of step (c) may be the same as in binding step (a) or the concentration may be different.

The concentration (w/v) of the poly(alkylene oxide) polymer in the reagent composition of step (c) is preferably lower than the concentration (w/v) of the poly(alkylene oxide) polymer in the binding mixture of step (a), thereby improving the purification results. Furthermore, also the concentration of the salt in the reagent composition of step (c) may be lower than the concentration of the salt in the binding mixture of step (a). These features, especially in combination, provide very stringent conditions in step (c), thereby ensuring an efficient elution of unwanted nucleic acid molecules that were despite the size-selective binding conditions nevertheless bound in step (a). The concentration of the poly(alkylene oxide) polymer and the salt can be lowered between step (a) and step (c) by the same ratio. This can be e.g. achieved by diluting the binding reagent comprising the poly(alkylene oxide) polymer and the salt with a dilution reagent, whereby the concentration of the polymer and the salt are lowered by the same ratio.

As is demonstrated in the examples, it is highly advantageous if the concentration (w/v) of the poly(alkylene oxide) polymer and, preferably also the concentration of the salt, in the reagent composition of step (c) is lower compared to the binding mixture provided in step (a). This in particular applies if the molecular weight of the poly(alkylene oxide) polymer, which preferably is a polyethylene glycol, is the same during binding step (a) and size-selective elution step (c). According to one embodiment, the concentration(s) of the poly(alkylene oxide) polymer and/or the salt in the reagent composition of step (c) is at least 90% of the concentration(s) of the poly(alkylene oxide) polymer and/or the salt in the binding mixture. E.g. the concentration(s) of the poly(alkylene oxide) polymer and/or the salt in the reagent composition of step (c) may be at least 93% or at least 95% of the concentration(s) of the poly(alkylene oxide) polymer and/or the salt in the binding mixture. As is demonstrated by the conditions of step (c) can be chosen such that non-target nucleic acids are efficiently depleted while the unwanted elution of target nucleic acid molecules is kept to a minimum.

As is shown in the examples, polymers of various molecular weights can be used in the size selective elution step (c). The concentration of the polymer in the reagent composition/reagent (c) can be adjusted to ensure that target nucleic acids remain bound to the solid phase, while the non-target nucleic acids are selectively eluted. As is demonstrated in the examples, it is advantageous to use a poly(alkylene oxide) polymer, preferably a polyethylene glycol, that has a molecular weight of at least 5000, such as at least 6000 or at least 8000 (such as e.g. PEG 8000) at least in step (c) and preferably also in step (a), as the size-selective elution results are good and robust. As disclosed herein, the same type of poly(alkylene oxide) polymer may be used in step (a) and step (c), preferably a polyethylene glycol, which may have the same molecular weight. According to a further embodiment, a poly(alkylene oxide) polymer, preferably a polyethylene glycol, is used in step (c) which has a higher or lower molecular weight compared to the poly(alkylene oxide) polymer, preferably a polyethylene glycol, that is used in binding step (a).

As disclosed herein, the binding conditions in step (a) are preferably established by adding a binding reagent that comprises the poly(alkylene oxide) polymer and the salt to the nucleic acid containing sample. The concentration (w/v) of the poly(alkylene oxide) polymer in the binding reagent that is added in step (a) may be higher than the concentration (w/v) of the poly(alkylene oxide) polymer in the reagent composition of step (c), respectively reagent (c). As disclosed herein, the concentration (w/v) of the poly(alkylene oxide) polymer in the binding mixture of step (a) is higher than the concentration (w/v) of the poly(alkylene oxide) polymer in the reagent composition of step (c). Furthermore, the concentration of the salt in the binding reagent that is added in step (a) is in one embodiment higher than the concentration of the salt in the reagent composition of step (c), respectively reagent (c). As disclosed herein, the concentration of the salt in the binding mixture of step (a) may be higher than the concentration of the salt in the reagent composition of step (c). According to one embodiment, the concentration of the poly(alkylene oxide) polymer and the salt in the binding reagent that is added in step (a) is higher than the concentration of the poly(alkylene oxide) polymer and the salt in the reagent composition of step (c), respectively reagent (c).

According to one embodiment, the reagent composition of step (c) is provided by mixing a reagent, comprising a poly(alkylene oxide) polymer and a salt (e.g. a binding reagent as disclosed herein), with a dilution reagent such as a dilution solution or dilution buffer. The reagent preferably comprises a poly(alkylene oxide) polymer and a salt as described before, in particular a polyethylene glycol and an alkali metal salt as described before. According to one embodiment, the dilution reagent comprises predominantly water. The dilution reagent may additionally comprise a buffering agent, which may be a buffering agent described in the present disclosure. According to a particular embodiment, the dilution reagent comprises Tris and EDTA (also referred to as TE buffer). The dilution reagent preferably does not comprise a poly(alkylene oxide) polymer and/or a salt. The reagent and the dilution reagent may be mixed by any method known in the art. Moreover, the reagent (e.g. the binding reagent used in step (a)) and the dilution reagent may be mixed at any ratio or factor suitable for forming a reagent composition of step (c), as described herein. A volume of the reagent (e.g. the binding reagent as described herein) may be diluted with an appropriate volume of the dilution reagent in order to prepare a reagent composition (c) as described herein. The ratio to be applied in particular depends on the concentration of the poly(alkylene oxide) polymer in the reagent that is diluted with the dilution solution and the concentration that is to be achieved in the reagent composition of step (c).

According to one embodiment, X volume of the reagent (preferably the binding reagent used in step (a)) is mixed with 1 volume of the dilution reagent, such as a TE buffer. X may be selected from any number, for instance X may lie in the range of 0.1 to 3, such as 0.2 to 2.5, 0.3 to 2.0, 0.4 to 1.8, 0.5 to 1.6, 0.6 to 1.4, or preferably 0.7 to 1.2. According to one embodiment, X is at least 0.3, such as at least 0.4, at least 0.5, at least 0.6 or at least 0.7. The binding reagent as disclosed may be used for preparing the reagent composition for step (c) by mixing with a dilution reagent. According to one embodiment, X volume of the binding reagent is mixed with 1 volume of the dilution reagent, wherein X lies in a range of 0.5 to 1.2, preferably 0.6 to 1.1, more preferably 0.7 to 1.05. Details of the binding reagent are described elsewhere.

As is described herein and demonstrated in the examples the use of a solid phase comprising carboxyl groups at the surface is particularly suitable and preferred in the context of the present invention. All disclosures described herein in the context of a solid phase in general, also specifically apply and refer to the use of a solid phase comprising carboxyl groups at the surface, such as carboxylated particles which preferably are magnetic carboxylated particles. The reagent composition/reagent (c) disclosed above is particularly suitable for use in combination with a solid phase that comprises carboxyl groups, such as e.g. carboxylated magnetic particles.

For contacting, the solid phase with the bound nucleic acids may be incubated and moved, e.g. immersed, suspended or agitated, within the reagent composition, respectively reagent (c). This is particularly feasible if using particles, preferably magnetic particles, for binding. The solid phase with the bound nucleic acids may be agitated, e.g. shaked, in the reagent to support the elution of the small non-target nucleic acid molecules.

Step (c) can furthermore be repeated. In this embodiment, the solid phase with the bound target nucleic acid molecules is preferably separated from the remaining reagent composition of step (c) which comprises eluted non-target nucleic acid molecules. The separated solid phase is then contacted again with a reagent composition/reagent (c) comprising a poly(alkylene oxide) polymer and a salt to selectively elute further non-target nucleic acid molecules that may still be bound to the solid phase. The solid phase may be contacted with the same reagent composition/reagent (c) that was used in the first size-selective elution step (c). According to one embodiment, step (c) is performed at least two times. As is demonstrated in the examples, repeating step (c) may further improve the results by even further removing small non-target nucleic acid molecules having a size below the desired cut-off from the solid phase. This is particularly advantageous if the starting material, i.e. the nucleic acid containing sample, comprises a high amount of small nucleic acid fragments below the cut-off value. E.g. as is demonstrated in the examples, repeating step (c) further improves the adapter removal even from highly adapter contaminated samples. Performing step (c) two or more times is therefore advantageous to remove large amounts of contaminating short DNA fragments such as e.g. adapter monomers or adapter-adapter ligation products during the preparation of a sequencing library. According to one embodiment, step (c) is performed twice.

### Step (d)

The bound target nucleic acid molecules may optionally be washed after performing step (c). Here, one or more washing steps can be performed. Even though this washing is optional, it is preferably performed in order to efficiently remove unbound components and impurities such as e.g. nucleotides and enzymes from previous reactions. This is particularly suitable if the nucleic acid containing sample was obtained during the preparation of a sequencing library. Furthermore, performing washing step(s) (d) is advantageous to remove traces of the poly(alkylene oxide) polymer and salt(s) used in steps (a) and (c).

Hence, the method of the present disclosure may have one or more of the following features:
washing step (d) is performed and comprises washing the bound nucleic acid molecules at least once with a wash solution that comprises an alkanol, preferably ethanol; and/or
washing step (d) is performed and the solid phase comprising the bound nucleic acid molecules is subsequently dried prior to elution step (e).

Thus, according to one embodiment, one or more washing steps are performed in (d) in order to further purify the bound target nucleic acid molecules. For this purpose, common suitable washing solutions may be used. A suitable washing solution removes impurities but not the target nucleic acid that is bound to the solid phase. According to one embodiment, the solution used for washing comprises at least one alcohol, preferably an alkanol. As alkanol, short chained branched or unbranched alcohols with preferably one to 5 carbon atoms can be used for washing, respectively can be used in the washing solution. Also mixtures of alcohols can be used. Suitable alcohols include but are not limited to methanol, ethanol, propanol, isopropanol and butanol. Preferably, isopropanol and/or ethanol are used in the washing solution. A further suitable washing solution which can be used alternatively or also in addition comprises an alcohol and a buffering agent. Suitable alcohols and buffering agents such as biological buffers are described above. Preferably, isopropanol or ethanol, most preferred ethanol is used in at least one washing step. Preferably, ethanol is used in a concentration of at least 60% (v/v), at least 70% (v/v), preferably at least 80% (v/v). A further suitable washing solution which can be used alternatively or optionally also in addition to the washing solutions described above comprises an alcohol but no salt. This allows to wash away salts. Preferably, isopropanol or ethanol, most preferred ethanol is used for washing. Preferably, the alcohol such as ethanol is comprised in a concentration of at least 50% v/v, at least 60% v/v, preferably at least 70% v/v. Preferably, the concentration lies in a range of 50% v/v to 100% v/v, more preferred 70% v/v to 100% v/v.

Residual alcohol that may be present after the washing step(s) in case an alcohol containing washing solution was used can be removed e.g. by air drying (e.g. suitable when working with a particulate solid phase) or by an additional centrifugation step. Respective methods and procedures are well-known in the prior art and thus, do not need any further description here.

### Step (e)

Step (e) comprises eluting the bound nucleic acid molecules from the solid phase. One or more elution steps may be performed in order to effectively release the size selected target nucleic acid molecules from the solid phase.

According to one embodiment, elution step (e) is performed by contacting the solid phase comprising the bound nucleic acid molecules with an elution solution.

Here, basically any elution solution can be used which effects desorption of the bound nucleic acid from the solid phase. Common elution solutions known to effectively elute nucleic acids such as DNA include but are not limited to water (e.g. deionized water), elution buffers such as TE-buffer and low-salt solutions which have a salt content of 150 mM or less, e.g. 100 mM or less, preferably 75 mM or less, 50 mM or less, 25 mM or less, 20 mM or less, 15 mM or less, 10 mM or less or are salt-free. Commercially available elution solutions are e.g. buffers EB and AE (Qiagen). The elution solution may e.g. comprise a buffering agent, in particular may comprise a biological buffer such as Tris, MOPS, HEPES, MES, BIS-TRIS, propane and others. The buffering agent may be present in a concentration of 150 mM or less, preferably 100 mM or less, more preferred 75 mM or less, 50 mM or less, 25 mM or less, 20 mM or less, 15 mM or less or 10 mM or less. According to one embodiment, the elution buffer has a pH value that is selected from pH 6 to pH 10, pH 7 to pH 9.5 and pH 7.5 to 9.0. Elution can be assisted by heating and/or shaking what is e.g. particularly feasible if a particulate solid phase is used for binding.

Preferably, an elution solution is used that does not interfere with the intended downstream application.

The present method for enriching target nucleic acids by size is precise and reproducible with respect to the size of the enriched target nucleic acid molecules and leads to high recovery rates. It is fast and cost-efficient because it does not require several “bind-wash-elute” cycles in order to purify the target nucleic acid molecules. The method according to the present disclosure is suitable for batch procedures. The method of the present invention does not need highly specific and expensive apparatuses. The method is advantageously suitable for automation. Here, it is favourable to use magnetic particles for providing the solid phase.

### Further Embodiments

Non-limiting preferred embodiments and applications of the method according to the present invention will be described further in the following. As disclosed herein, the size selective nucleic acid isolation method according to the present invention is in particular suitable for enriching target nucleic acid molecules having a desired size above a certain cut-off value and/or having a size that lies within a certain size range (determined by an upper and lower cut-off value) from a mixed population of nucleic acid molecules having different lengths/sizes. The method is in particular suitable for removing and thus depleting non-target DNA molecules which have a size below a certain cut-off value by binding and thus isolating target DNA molecules having a desired minimum size above the cut-off value from a DNA containing sample.

According to one embodiment, the method comprises
(a) preparing a binding mixture comprising
   the nucleic acid containing sample,
   a poly(alkylene oxide) polymer having a molecular weight that lies in a range of 3000 to 25000, preferably 4000 to 25000, such as 6000 to 25000, wherein preferably, the poly(alkylene oxide) polymer is a polyethylene glycol and
   a salt, wherein the salt is an alkali metal salt, preferably selected from sodium chloride and potassium chloride, and binding nucleic acid molecules to the solid phase comprising a functional group (preferably carboxylated magnetic particles), wherein the bound nucleic acid molecules comprise target nucleic acid molecules having a size above a cut-off value, optionally wherein under the used binding conditions the smaller non-target nucleic acid molecules having a size below a cut-off value do not bind to the solid phase or bind to a lesser extent to the solid phase compared to the longer target nucleic acid molecules;
(b) separating the solid phase with the bound nucleic acid molecules from the remaining sample;
(c) contacting, e.g. suspending, the solid phase with the bound nucleic acid molecules at least once with a reagent composition comprising
   a poly(alkylene oxide) polymer having a molecular weight that lies in a range of 3000 to 25000, preferably 5000 to 25000, such as 6000 to 20000 or 6000 to 16000, wherein preferably, the poly(alkylene oxide) polymer is a polyethylene glycol, and
   a salt, wherein the salt is an alkali metal salt, preferably a non-chaotropic salt, more preferably selected from sodium chloride and potassium chloride, to selectively elute non-target nucleic acid molecules (having a size below the desired cut-off), wherein the concentration (w/v) of the poly(alkylene oxide) polymer in the reagent composition of step (c) is lower than the concentration (w/v) of the poly(alkylene oxide) polymer in the binding mixture of step (a);
(d) optionally washing the bound target nucleic acid molecules; and
(e) eluting the bound target nucleic acid molecules from the solid phase.

As disclosed herein, the cut-off value in the binding step (a) and the size selective elution step (c) may be adjusted by adjusting the concentration (w/v) of the poly(alkylene oxide) polymer. Furthermore, as disclosed herein, using a higher molecular weight polymer (e.g. at least 5000, at least 6000 or at least 8000 such as e.g. PEG 8000) at least in step (c) is advantageous for the robustness of the performance. The same type of poly(alkylene oxide) polymer may be used in step (a) and step (c), which preferably is a polyethylene glycol, and which may have the same molecular weight.

According to one embodiment, the method comprises
(a) preparing a binding mixture comprising
   the nucleic acid containing sample,
   a poly(alkylene oxide) polymer in a concentration of at least 5% (w/v), preferably at least 7% (w/v), wherein the poly(alkylene oxide) polymer has a molecular weight that lies in a range of 3000 to 30000, preferably 5000 to 25000, such as 6000 to 20000 or 6000 to 16000, wherein preferably, the poly(alkylene oxide) polymer is a polyethylene glycol and
   a salt in a concentration of at least 500 mM, preferably at least 800 mM, wherein the salt is an alkali metal salt, preferably selected from sodium chloride and potassium chloride, and binding nucleic acid molecules to the solid phase comprising a functional group (preferably carboxylated magnetic particles), wherein the bound nucleic acid molecules comprise target nucleic acid molecules, optionally wherein under the used binding conditions the smaller non-target nucleic acid molecules having a size below a cut-off value do not bind to the solid phase or bind to a lesser extent to the solid phase compared to the longer target nucleic acid molecules;
(b) separating the solid phase with the bound nucleic acid molecules from the remaining sample;
(c) contacting the solid phase with the bound nucleic acid molecules at least once with a reagent composition comprising a poly(alkylene oxide) polymer, preferably a polyethylene glycol, and a salt to selectively elute non-target nucleic acid molecules (having a size below the cut-off), wherein the concentration (w/v) of the poly(alkylene oxide) polymer in the reagent composition of step (c) is lower than the concentration (w/v) of the poly(alkylene oxide) polymer in the binding mixture of step (a);
(d) optionally washing the bound nucleic acid molecules; and
(e) eluting the bound nucleic acid molecules from the solid phase.

According to one embodiment, the method comprises
(a) preparing a binding mixture comprising
   the nucleic acid containing sample, wherein the contained nucleic acids comprise or consist of DNA molecules of different sizes,
   a poly(alkylene oxide) polymer, preferably a polyethylene glycol, and
   a salt and binding DNA molecules to a solid phase, wherein the solid phase is provided by carboxylated magnetic particles, wherein the bound DNA molecules comprise target DNA molecules, preferably wherein under the used binding conditions the smaller non-target DNA molecules having a size below the cut-off value for the target DNA molecules do not bind to the solid phase or bind to a lesser extent to the solid phase compared to the longer target DNA molecules;
(b) separating the solid phase with the bound DNA molecules from the remaining sample;
(c) contacting the solid phase with the bound DNA molecules at least once with a reagent composition comprising a poly(alkylene oxide) polymer and a salt to selectively elute non-target nucleic acid molecules having a size below the cut-off, wherein the concentration (w/v) of the poly(alkylene oxide) polymer in the reagent composition of step (c) is lower than the concentration (w/v) of the poly(alkylene oxide) polymer in the binding mixture of step (a);
(d) optionally washing the bound target DNA molecules; and
(e) eluting the bound target DNA molecules from the carboxylated magnetic particles.

According to one embodiment, the method comprises
(a) preparing a binding mixture comprising
   the nucleic acid containing sample, wherein the contained nucleic acids comprise or consist of DNA molecules of different sizes,
   a poly(alkylene oxide) polymer, preferably a polyethylene glycol, having a molecular weight that lies in a range of 3000 to 25000, preferably in a range of 4000 to 25000, such as in a range of 6000 to 20000, and
   a salt, wherein the salt is an alkali metal salt, preferably selected from sodium chloride and potassium chloride, and binding DNA molecules to a solid phase, wherein the solid phase is provided by carboxylated magnetic particles, wherein the bound DNA molecules comprise target DNA molecules, preferably wherein under the used binding conditions the smaller non-target DNA molecules having a size below the cut-off value for the target DNA molecules do not bind to the solid phase or bind to a lesser extent to the solid phase compared to the longer target DNA molecules;
(b) separating the solid phase with the bound DNA molecules from the remaining sample;
(c) contacting the solid phase with the bound DNA molecules at least once with a reagent composition comprising a poly(alkylene oxide) polymer and a salt to selectively elute non-target nucleic acid molecules having a size below the cut-off, wherein the concentration (w/v) of the poly(alkylene oxide) polymer in the reagent composition of step (c) is lower than the concentration (w/v) of the poly(alkylene oxide) polymer in the binding mixture of step (a);
(d) optionally washing the bound target DNA molecules; and
(e) eluting the bound target DNA molecules from the carboxylated magnetic particles.

According to one embodiment, the method comprises
(a) contacting a binding reagent that comprises a poly(alkylene oxide) polymer and a salt with a nucleic acid containing sample, wherein the contained nucleic acids comprise or consist of DNA molecules of different sizes, thereby preparing a binding mixture comprising
   the nucleic acid containing sample,
   the poly(alkylene oxide) polymer, preferably a polyethylene glycol, in a concentration of at least 7% (w/v), wherein the poly(alkylene oxide) polymer has a molecular weight that lies in a range of 3000 to 25000, preferably in a range of 5000 to 25000, such as in a range of 6000 to 20000, and
   the salt in a concentration of at least ≥500 mM, preferably at least 800 mM, wherein the salt is an alkali metal salt, preferably selected from sodium chloride and potassium chloride, and binding DNA molecules to a solid phase, wherein the solid phase is provided by carboxylated magnetic particles, wherein the bound DNA molecules comprise target DNA molecules, preferably wherein under the used binding conditions the smaller non-target DNA molecules having a size below the cut-off value for the target DNA molecules do not bind to the solid phase or bind to a lesser extent to the solid phase compared to the longer target DNA molecules;
(b) separating the solid phase with the bound DNA molecules from the remaining sample;
(c) contacting the solid phase with the bound DNA molecules at least once with a reagent composition comprising a poly(alkylene oxide) polymer, preferably a polyethylene glycol, and a salt to selectively elute non-target nucleic acid molecules having a size below the cut-off, wherein the concentration (w/v) of the poly(alkylene oxide) polymer in the reagent composition of step (c) is lower than the concentration (w/v) of the poly(alkylene oxide) polymer in the binding mixture of step (a);
(d) optionally washing the bound target DNA molecules; and
(e) eluting the bound target DNA molecules from the carboxylated magnetic particles.

As disclosed herein, the method is particularly suitable for isolating adapter ligated DNA molecules as target DNA molecules from a DNA containing sample which is an adapter ligation sample. The size-selective method of the invention efficiently removes adapter monomers and adapter-adapter ligation products, thereby providing pure adapter ligated DNA molecules (e.g. for a sequencing library) with high yield. According to one embodiment the method accordingly comprises
(a) preparing a binding mixture comprising
   the adapter ligation sample,
   a poly(alkylene oxide) polymer, preferably a polyethylene glycol, and
   a salt and binding nucleic acid molecules to a solid phase, wherein the bound nucleic acid molecules comprise adapter ligated DNA molecules, optionally wherein under the used binding conditions the smaller adapter monomers and adapter-adapter ligation products having a length below a cut-off value do not bind to the solid phase or bind to a lesser extent to the solid phase compared to the longer adapter ligated DNA molecules;
(b) separating the solid phase with the bound DNA molecules from the remaining sample;
(c) contacting the solid phase with the bound DNA molecules at least once with a reagent composition comprising a poly(alkylene oxide) polymer, preferably a polyethylene glycol, and a salt to selectively elute adapter monomers and adapter-adapter ligation products;
(d) optionally washing the bound adapter ligated DNA molecules; and
(e) eluting the bound adapter ligated DNA molecules from the solid phase.

According to one embodiment said method comprises
(a) contacting a binding reagent that comprises a poly(alkylene oxide) polymer and a salt with a DNA containing sample which is an adapter ligation sample, thereby preparing a binding mixture comprising
   the adapter ligation sample,
   the poly(alkylene oxide) polymer, preferably a polyethylene glycol, in a concentration of at least 7% (w/v), wherein the poly(alkylene oxide) polymer has a molecular weight that lies in a range of 3000 to 30000, preferably in a range of 5000 to 25000, such as 6000 to 20000, and
   the salt in a concentration of at least ≥500 mM, preferably at least 800 mM, wherein the salt is an alkali metal salt, preferably selected from sodium chloride and potassium chloride,
   and binding DNA molecules to a solid phase comprising a functional group, wherein the bound DNA molecules comprise adapter ligated DNA molecules and wherein the solid phase is preferably provided by carboxylated magnetic particles, optionally wherein under the used binding conditions the smaller adapter monomers and adapter-adapter ligation products having a length below a cut-off value do not bind to the solid phase or bind to a lesser extent to the solid phase compared to the longer adapter ligated DNA molecules;
(b) separating the solid phase with the bound DNA molecules from the remaining sample;
(c) contacting the solid phase with the bound DNA molecules at least once with a reagent composition comprising
   a poly(alkylene oxide) polymer having a molecular weight that lies in a range of 3000 to 30000, preferably in a range of 5000 to 25000, such as 6000 to 20000, wherein preferably, the poly(alkylene oxide) polymer is a polyethylene glycol, and
   a salt, wherein the salt is an alkali metal salt, preferably selected from sodium chloride and potassium chloride, to selectively elute adapter monomers and adapter-adapter ligation products, wherein preferably, the concentration (w/v) of the poly(alkylene oxide) polymer in the reagent composition of step (c) is lower than the concentration (w/v) of the poly(alkylene oxide) polymer in the binding mixture of step (a);
(d) optionally washing the bound adapter ligated DNA molecules; and
(e) eluting the bound adapter ligated DNA molecules from the carboxylated magnetic particles.

According to one embodiment, said method comprises
(a) contacting a binding reagent, preferably as defined in claim 9 d), with a DNA containing sample which is an adapter ligation sample, thereby preparing a binding mixture comprising
   the adapter ligation sample,
   the polyethylene glycol in a concentration of at least 7% (w/v), preferably at least 8%, and
   the salt in a concentration of at least 750 mM, preferably at least 800 mM, more preferably at least 900 mM, and binding adapter ligated DNA molecules to a solid phase comprising a functional group, wherein the solid phase is preferably provided by carboxylated magnetic particles, wherein under the used binding conditions the smaller adapter monomers and adapter-adapter ligation products do not bind to the solid phase or bind to a lesser extent to the solid phase compared to the longer adapter ligated DNA molecules;
(b) separating the solid phase with the bound DNA molecules from the remaining sample;
(c) providing a reagent composition, preferably as defined in claim 10 e), by mixing the binding reagent used in step (a) with a dilution solution and contacting the separated solid phase comprising the bound DNA molecules at least once with the provided reagent composition to selectively elute adapter monomers and adapter-adapter ligation products from the solid phase while the majority of the adapter ligated DNA molecules remain bound to the solid phase, wherein the concentration (w/v) of the polyethylene glycol in step (c) is lower than the concentration (w/v) of the polyethylene glycol in the binding mixture of step (a);
(d) optionally washing the bound adapter ligated DNA molecules; and
(e) eluting the bound adapter ligated DNA molecules from the carboxylated magnetic particles.

Suitable and preferred embodiments for the individual steps, in particular steps (a) and (c) as well as suitable and preferred binding reagents and reagents (c) were described above and can be used in the methods described herein as further embodiments.

According to one embodiment, at least steps (b) to (e) are performed on a sample processing system. The sample processing system has one or more of the following characteristics:
a) it is an automated system;
b) it does not comprise a pipetting unit;
c) it comprises at least two magnets for processing magnetic particles.

According to one embodiment, an automated system is used that does not require a pipetting unit. A corresponding system is e.g. disclosed in US2016/0202157, herein incorporated by reference, and is commercially available as Extractman®.

As discussed above, the method according to the present invention is in particular suitable for size selection in the context of next generation sequencing. The preparation of a sequencing library suitable for next generation sequencing usually is a multi-step process wherein at different stages of said process a size-selection of the provided DNA molecules may be performed.

At which stage of said process a size selection is performed also depends on the library preparation method used. The size selection method according to the present invention is in particular suitable for use in the context of preparing a sequencing library, as it allows the separation of DNA fragments with only small differences in size, e.g. as described herein, the removal of unwanted adapter dimers (approx. 120 bp) from the desired DNA fragments (e.g. at least 200 bp, at least 300 bp, at least 400 bp and larger) in library construction protocols for next generation sequencing applications.

A sequencing library which is suitable for massive parallel sequencing and accordingly, is suitable for next generation sequencing can be prepared using methods known in the prior art. The preparation of a respective sequencing library often involves the generation of a plurality of double-stranded, linear DNA fragments from a nucleic acid containing sample. For example, DNA, such a genomic DNA or cDNA, can be fragmented for example by shearing, such as sonification, hydro-shearing, ultrasound, nebulization or enzymatic fragmentation, in order to provide DNA fragments that are suitable for subsequent sequencing. A separate fragmentation step is not required if the provided DNA has a fragment profile of an acceptable length, as it is e.g. the case when processing extracellular DNA.

The length of the fragments can be chosen based on the read length of the next generation sequencing platform that is subsequently used for sequencing. Usually, the obtained fragments have an average length of 1500 bp or less, e.g. 1000 bp or less, 750 bp or less, 600 bp or less or e.g. 500 bp or less as this corresponds to the sequencing capacity of most current next generation sequencing platforms. The obtained fragments may have an average length that predominantly lies in a range of 50 bp to 1000 bp, such as 75 bp to 900 bp, 100 bp to 850 bp, 110 bp to 800 bp, 115 bp to 750 bp, 120 bp to 700 bp, 125 bp to 650 bp, 130 bp to 600 bp, 135 bp to 550 bp, 140 bp to 500 bp and 145 bp to 450 bp. Respective fragment sizes are particularly suitable for genomic DNA, also considering that the size of an exon is approx. 150 bp to 200 bp in length and respective short DNA fragments can be efficiently sequenced using common next generation sequencing platforms. However, also longer fragments can be useful, e.g. if using next generation sequencing methods which allow longer sequence reads.

According to one embodiment, the fragmented DNA is repaired after fragmentation and end polished using methods known in the prior art, thereby providing DNA fragments having blunt ends. In such methods which are well-known in the prior art, overhangs resulting from the fragmentation process are converted into blunt ends. As the respective methods are well-known in the prior art, they do not need any detailed description herein.

According to one embodiment, the size selective nucleic acid enrichment method according to the invention is performed after DNA fragments were obtained, preferably after the fragmented DNA was end polished to provide DNA fragments having blunt ends. Size selection at this stage allows e.g. to eliminate very short DNA fragments which do not have the appropriate length for subsequent sequencing. According to one embodiment, the method according to the first aspect is performed at this stage. As discussed above, the cut-off value for DNA binding can be adjusted by appropriate choice of the concentration of the poly(alkylene oxide) polymer in the binding mixture that is prepared in step (a) of the present method. The present method allows to enrich DNA fragments that substantially have a size within a defined size range, while very efficiently depleting smaller DNA fragments due to intermediate step (c). Performing the method of the present disclosure at this stage is therefore advantageous.

According to one embodiment, after end-repair and optionally size selection, an overhang is added to the 3′ ends of the blunt end fragments. Preferably, a single nucleotide overhang is added. E.g. a single “A” nucleotide can be added using methods well-known in the prior art. This is also referred to as “A tailing”. A respective nucleotide overhang prevents the fragments from ligating to one another during the subsequent adapter ligation reaction. E.g. a corresponding single “T” or other complementary overhang can be provided at the 3′ end of the adapters to provide a complementary overhang for ligating the adapters to the DNA fragment. This ensures a low rate of chimera (concatenated template) formation. However, also other strategies are known in the prior art to ensure proper ligation of sequencing adapters. E.g. also blunt end adapters can be ligated.

According to one embodiment, adapters are ligated to the 5′ and/or 3′ ends of the obtained DNA fragments, preferably at both ends of the DNA fragments. The specific design of the adapters depends on the next generation sequencing platform to be used and for the purposes of the present invention, basically any adapters used for preparing sequencing libraries for next generation sequencing can be used. The adapter sequences provide a known sequence composition allowing e.g. subsequent library amplification and/or sequencing primer annealing. As adapters, double-stranded or partially double-stranded nucleic acids of known sequence can be used. The adapters may have blunt ends, cohesive ends with 3′ or 5′overhangs, may be provided by Y shaped adapters or by stem-loop shaped adapters. Y shaped adapters are e.g. described in U.S. Pat. No. 7,741,463 and stem-loop shaped adapters are e.g. described in US2009/0298075. The adapters may have a length of at least 7, at least 10, preferably at least 15 bases. The adapter length may lie in a range of 10 to 100 bases, preferably 15 to 75 bases, more preferred 20 to 60 bases. Either the same or different adapters can be used for the 3′ and 5′ end of the DNA fragments. Using the same type of adapter for both ends, such as e.g. a Y shaped or a stem-looped shaped adapter, has the advantage that no fragments are lost during library preparation due to adapter mispairing which is an advantage when working with low amounts of DNA.

According to one embodiment, a sequencing library is prepared which comprises randomly fragmented double stranded DNA molecules which are ligated at their 3′ and 5′ end to adapter sequences. The adapters provide a known sequence and thus provide a known template for amplification and/or sequencing primers. Optionally, the adapters may also provide an individual index thereby allowing the subsequent pooling of two or more target enriched sequencing libraries prior to sequencing.

To ensure an efficient adapter ligation, the adapters are usually used in excess during the adapter ligation step. Thus, after adapter ligation, a DNA containing sample is provided which comprises DNA molecules that are flanked by adapters in addition to unligated adapter monomers and adapter-adapter ligation products such as adapter dimers. Unligated adapter monomers and adapter-adapter ligation products are usually comprised in large amounts in the sample that is obtained after the adapter ligation process. To remove unligated adapter monomers and adapter-adapter ligation products as well as enzymes and other contaminants from the adapter ligation sample, it is preferred to perform a size selective DNA enrichment using the method according to the present invention. The cut-off value for the target DNA molecules is chosen such that it lies above the size of unligated adapter monomers and above the size of expected adapter-adapter ligation products. This ensures that unligated adapter monomers and unwanted adapter-adapter ligation products are substantially not captured during the size selective DNA binding step (a) and if bound, are efficiently eluted in step (c) and thereby are efficiently depleted from the isolated target DNA, which predominantly consists of DNA molecules having a size above the cut-off value as is demonstrated in the examples. The present method is particularly advantageous for this purpose, because the cut-off value can be precisely adjusted by varying the concentration of the poly(alkylene oxide) polymer in the binding mixture and/or step (c).

Thus, according to one embodiment, the method of the invention is used for isolating adapter ligated DNA molecules as target DNA molecules from a DNA containing sample which is an adapter ligation sample and for removing adapter monomers and adapter-adapter ligation products, wherein adapter ligated DNA molecules are separated from unligated adapter monomers and adapter-adapter ligation products based on the larger size of the adapter ligated DNA molecules. The cut-off value lies above the size of adapter monomers and above the size of expected adapter-adapter ligation products. According to one embodiment, the cut-off value lies at least 50 nt, preferably at least 75 nt or at least 100 nt above the size of expected adapter-adapter ligation product(s).

According to one embodiment, target DNA fragments are enriched after adapter ligation and size selection as described above by amplification, preferably PCR amplification. Such enrichment step is optional, but preferred for some applications. E.g. an amplification reaction such as a PCR amplification can be used to selectively enrich those DNA fragments that have adapter molecules on both ends and to amplify the amount of DNA in the library. According to one embodiment, the PCR is performed with one or more primers that anneal to the adapters. Respective amplification steps are well known in the prior art and thus, do not need any detailed description here.

According to one embodiment, a size-selective DNA enrichment according to the method of the present disclosure is performed after amplification. The cut-off value is again chosen such that primers, unligated adapter monomers and adapter-adapter ligation products that might have been present during amplification and might have been amplified, are not captured during DNA binding. According to one embodiment, the same cut-off value is used as was used when performing a size selective DNA enrichment after the adapter ligation step.

Thus, according to one embodiment, the method according to the present invention comprises amplifying the isolated, size selected adapter ligated double stranded DNA molecules to provide an enriched sequencing library. According to one embodiment, the size selection method according to the first aspect is performed after amplification. After amplification enrichment, which may be followed by a DNA size selection according to the present invention, the sequencing library is ready for use. Optionally, the prepared sequencing library can be validated, quantified and/or quality controls can be performed to verify the size of the obtained adapter ligated fragments, respectively PCR enriched fragments.

Suitable general methods for preparing sequencing libraries are also described in the prior art, see e.g. Metzker, 2011, Voelkerding, 2009, WO12/003374, and the further prior art discussed in the background and can be combined with the size selective DNA enrichment method according to the present invention.

The method of the present disclosure can in particular be used to purify desired DNA molecules having a size above a certain cut-off value (desired DNA molecules are also referred to herein as “target” DNA molecules) by binding said target DNA molecules in step (a) to the solid phase. The bound target DNA molecules are separated in step (b). This allows to perform a “one-sided” size selection, namely for target DNA molecules having a certain minimum size as it is determined by the cut-off value. However, a maximum size of the target DNA molecules (determined by an upper cut-off value) is not defined. This embodiment is e.g. suitable for isolating and hence separating target DNA molecules having a size above a desired cut-off value from smaller DNA molecules that represent undesired contaminations such as e.g. oligonucleotides, primers, shorter DNA fragments such as adapters, adapter dimers etc.

The method can also be performed in order to size selectively enrich and thereby isolate such DNA fragments having a length within a certain size range that is determined by an upper cut-off value and a lower cut-off value from a DNA containing sample, e.g. by performing at least two size selective binding steps. According to one embodiment, target nucleic acid molecules having a size within a size range that is determined by an upper and a lower cut-off value are purified. The lower cut-off value preferably corresponds to the cut-off value described in detail above. The upper cut-off value can be chosen in accordance with the desired size range and the nucleic acid molecules present in the nucleic acid containing sample. According to one embodiment, the method comprises a poly(alkylene oxide) polymer and salt based pre-binding step (x) that is performed prior to step (a) and wherein nucleic acid molecules having a size above the upper cut-off value are bound to the solid phase. In step (x), the poly(alkylene oxide) polymer concentration is lower than in step (a). Thereby, longer nucleic acid molecules having a size above the upper cut-off value are bound to the solid phase and can be removed together with the solid phase, while the target nucleic acids remain in the binding mixture and can then be purified by performing steps (a) to (e) of the method according to the present invention. The longer nucleic acid molecules having a size above the upper cut-off value that were bound to a solid phase in step (x) can after separation be either discarded or may be further processed. The upper cut-off value can be adjusted by adjusting the concentration of the at least one poly(alkylene oxide) polymer. Advantageously, the upper and the lower cut-off value can be easily adjusted by adding appropriate amounts of the binding buffer to establish size selective binding conditions in the binding mixture. The same type of solid phase can be used in steps (x) and (a).

### METHOD FOR PREPARING SEQUENCING LIBRARY

According to a second aspect of the present disclosure, a method for preparing a sequencing library is provided that is preferably suitable for massive parallel sequencing, wherein said method comprises
A) optionally fragmenting DNA and optionally end repairing DNA fragments to provide a sample comprising blunt end DNA fragments of different sizes;
B) performing an adapter ligation step to provide a sample comprising double-stranded DNA molecules that are flanked 5′ and/or 3′ by adapters;
C) separating adapter ligated double-stranded DNA molecules from unligated adapter monomers and adapter-adapter ligation products based on the larger size of the adapter ligated double stranded DNA molecules using the method according to the first aspect;
D) optionally amplifying adapter ligated DNA molecules.

This aspect provides a sequencing library comprising adapter ligated DNA fragments having an appropriate minimal length. As discussed above, preferably, adapters are provided at the 3′ end and the 5′ end of the DNA fragments. Furthermore, the size selection step performed in step C) efficiently removes adapter monomers and adapter-adapter ligation products as well as other contaminants from the ligation reaction in a fast and cost-effective manner. This is an important improvement. In contrast to prior art methods, it is not required to repeat the whole “bind-wash-elute” procedure of size selection in order to ensure an efficient removal of substantially all adapter monomers and adapter-adapter ligation products and to increase the size selection stringency. Instead, the method according to the first aspect achieves the same or an even better quality by performing at least one treatment step (c) which results in a selective elution of adapter monomers or adapter-adapter ligation products that might have/are bound in binding step (a) besides size selective binding conditions being used thereby ensuring that highly pure adapter ligated DNA molecules above the cut-off value are provided with high yield. The method is fast, reliable, cost-efficient and provides a sequencing library of high quality. Furthermore, said method can be easily integrated into existing sequencing library preparation methods.

A single NGS run usually produces enough reads to sequence several target enriched sequencing libraries at once. Therefore, pooling strategies and indexing approaches are a practical way to reduce the per sample cost. Respective multiplexing strategies can also be used in conjunction with the teaching of the present invention. Features enabling multiplexing can be incorporated in different stages of the enrichment process. According to one embodiment, the sequencing library is generated by using adapters containing specific sequence motifs for library labelling and differentiation (“barcoded” or “index” adapters). Each sequencing library is provided with individual und thus library specific adapters which provide a library specific sequence. Preferably, each adapter comprises besides the index region a common universal region which provides a known template for PCR primers and/or sequencing primers that can be used on all libraries. After the target enriched sequencing libraries were obtained, they can be pooled and sequenced in a single run. Providing the DNA fragments of the sequencing library with respective index adapters thus allows subsequently sequencing several target enriched sequencing libraries in the same sequencing run because the sequenced fragments can be distinguished based on the library specific sequence of the index adapters. After sequencing, the individual sequences belonging to each library can be sorted via the library specific index which is then found in the obtained sequence. Respective index approaches are known in the prior art and index adapters are also commercially available and are for example provided in the TruSeq® DNA sample prep kits which are suitable for use in the Illumina platform.

According to one embodiment, the sequencing library comprises the double-stranded DNA molecules in an overall amount of 2 μg or less, e.g. 1.5 μg or less, 1 μg or less, 0.75 μg or less or 0.5 μg or less. Methods are furthermore available to prepare sequencing libraries in a nanogram range, e.g. 1 ng to 500 ng. According to one embodiment, the sequencing library comprises the double-stranded DNA molecules in an overall amount of at least 0.05 μg, at least 0.1 μg, 0.25 μg or at least 0.5 μg. The method according to the present invention not only enables a size-selective DNA isolation but also ensures an efficient capture of DNA molecules having the desired size and provides the purified target DNA molecules with high yield. This is an important advantage, because in many cases, the sequencing library comprises the DNA in low amounts and an unwanted loss of DNA material should be prevented during the preparation of the sequencing library.

Nucleic acids such as DNA and/or RNA can be isolated from a sample of interest according to methods known in the prior art to provide the starting material for preparing the sequencing library. RNA is usually first transcribed into cDNA prior to preparing the sequencing library.

As discussed above, sequencing is preferably performed on a next generation sequencing platform. In NGS, sequencing is usually performed by repeated cycles of polymerase-mediated nucleotide extensions or, in one common format, by iterative cycles of oligonucleotide ligation. After obtaining the sequencing library using the method according to the present invention, clonal separation of single molecules and subsequent amplification is performed by in vitro template preparation reactions like emulsion PCR (pyrosequencing from Roche 454, semiconductor sequencing from Ion Torrent, SOLiD sequencing by ligation from Life Technologies, sequencing by synthesis from Intelligent Biosystems), bridge amplification on the flow cell (e.g. Solexa/Illumin(a), isothermal amplification by Wildfire technology (Life Technologies) or rolonies/nanoballs generated by rolling circle amplification (Complete Genomics, Intelligent Biosystems, Polonator). Sequencing technologies like Heliscope (Helicos), SMRT technology (Pacific Biosciences) or nanopore sequencing (Oxford Nanopore) allow direct sequencing of single molecules without prior clonal amplification. Suitable NGS methods and platforms that can be used were also described in the background of the present invention and it is referred to the respective disclosure. The sequencing can be performed on any of the respective platforms.

### Kit

According to a third aspect, a kit is provided for the size selective enrichment of target nucleic acid molecules having a size above a desired cut-off value from a nucleic acid containing sample, comprising
(a) a binding reagent comprising at least one poly(alkylene oxide) polymer and at least one salt;
(b) magnetic particles for binding target nucleic acid molecules in the presence of binding reagent (a);
(c) a reagent comprising at least one poly(alkylene oxide) polymer and at least one salt and/or a dilution reagent for preparing the reagent (c) by combining the dilution reagent with the binding reagent;
(d) optionally at least one washing solution; and
(e) optionally an elution solution,
wherein the concentration of the poly(alkylene oxide) polymer in the binding reagent (a) is higher than the concentration of the poly(alkylene oxide) polymer in the reagent (c).

Details regarding the binding reagent, in particular suitable and preferred binding reagent components, binding reagent component concentrations, as well as details regarding the solid phase, reagent (c), the washing solution and the elution solution were described in detail above in conjunction with the method according to the first aspect. It is referred to the above disclosure which also applies here. Non-limiting selected embodiments are again described subsequently.

Suitable and preferred types and concentrations for the poly(alkylene oxide) polymer in the binding reagent were described above and it is referred to the above disclosure. Preferably, the poly(alkylene oxide) polymer is a poly(ethylene oxide) polymer, preferably a polyethylene glycol, more preferably unsubstituted polyethylene glycol.

Suitable and preferred types and concentrations for the salt in the binding reagent, which preferably is an alkali metal salt, preferably an alkali metal halide, have been described above and it is referred to the above disclosure. The salt is preferably selected from sodium chloride, potassium chloride, lithium chloride and cesium chloride, more preferably the salt is sodium chloride.

Specific examples for binding reagents are furthermore disclosed in conjunction with the method according to the present invention and also in the claims and these binding reagents can be included in the kit according to the third aspect.

Details regarding reagent (c) which comprises at least one poly(alkylene oxide) polymer and at least one salt and may be used in method step (c) to remove nucleic acid molecules having a size below the cut-off were described in detail above in conjunction with the method according to the present invention and also in the claims and it is referred to the above disclosure. Any one of these reagents (c) can be included in the kit according to the third aspect. As described above, reagent (c) may also be created by adding a dilution reagent (e.g. TE buffer) to the binding reagent (a) to thereby freshly prepare reagent (c). The dilution reagent may be mixed with an appropriate volume of the binding reagent to prepare reagent (c). The kit may comprise the dilution reagent as kit component. Details of the dilution reagent, e.g. a dilution solution, and mixing ratios were described above and it is referred to this disclosure.

Suitable and preferred embodiments of the solid phase were also described in conjunction with the method according to the invention and it is referred to the above disclosure. As described above, the solid phase preferably provides a carboxylated surface. Particularly preferred is the use of carboxylated magnetic particles. In one embodiment, the solid phase is comprised in the binding reagent (a).

Furthermore, the kit may comprise instructions and/or information for use. E.g. the kit may comprise instructions and/or information regarding the cut-off value that is achieved when mixing a certain volume of the binding buffer with a certain volume of the nucleic acid containing sample and/or the cut-off value(s) that are achieved if the nucleic acid containing sample is mixed in a certain ratio with the binding reagent. If two or more binding reagents are comprised in the kit that differ in the concentration of the poly(alkylene oxide) polymer, the kit may provide information which cut-off value is achieved when using a certain binding buffer comprised in the kit. Thus, the present invention also provides a kit which allows the flexible adjustment of the cut-off value e.g. by mixing a certain volume of the binding buffer and a certain volume of the nucleic acid containing sample.

A respective kit can be in particular used in the method according to the first aspect.

### Use

According to a fourth aspect, the present disclosure is directed to the use of a kit according to the third aspect in a method according to the first or second aspect. Specifically, the present disclosure provides a kit as defined in any one of claims 23 to 24 in a method as defined in any one of claims 1 to 22. Details regarding these aspects are described above and in the claims it is referred to the respective disclosure.

This invention is not limited by the exemplary methods and materials disclosed herein, and any methods and materials similar or equivalent to those described herein can be used in the practice or testing of embodiments of this invention. Numeric ranges are inclusive of the numbers defining the range. The headings provided herein are not limitations of the various aspects or embodiments of this invention which can be read by reference to the specification as a whole.

The term “solution” as used herein in particular refers to a liquid composition, preferably an aqueous composition. It may be a homogenous mixture of only one phase but it is also within the scope of the present invention that a solution comprises solid constituents such as e.g. precipitates.

As used in the subject specification and claims, the singular forms “a”, “an” and “the” include plural aspects unless the context clearly dictates otherwise. Thus, for example, reference to “a poly(alkylene oxide) polymer” includes a single type of poly(alkylene oxide) polymer, as well as two or more poly(alkylene oxide) polymers. Likewise, reference to “a salt”, “a buffering agent” and the like includes single entities and combinations of two or more of such entities. Reference to “the disclosure” and “the invention” and the like includes single or multiple aspects taught herein; and so forth. Aspects taught herein are encompassed by the term “invention”.

The solid phase is not considered when determining the concentrations of the components, such as the poly(alkylene oxide) polymer or the salt in the binding mixture.

According to one embodiment, subject matter described herein as comprising certain steps in the case of methods or as comprising certain ingredients in the case of compositions, solutions and/or buffers refers to subject matter consisting of the respective steps or ingredients. It is preferred to select and combine preferred embodiments described herein and the specific subject-matter arising from a respective combination of preferred embodiments also belongs to the present disclosure.

### EXAMPLES

The effectiveness of the method according to the present disclosure in removing non-target DNA fragments below a cut-off and for enriching target DNA molecules having a size above the cut-off value with good yield from a DNA containing sample was compared with the AMPure XP protocol, as state of the art reference protocol. Polyethylene glycol (PEG) was used as poly(alkylene oxide) polymer for size selection in all examples.

### I. Material and Methods

### 1. Preparation of Nucleic Acid Containing Sample as Starting Material

As nucleic acid containing sample, adapter-dimer contaminated sequencing libraries were prepared to assess the effectiveness of adapter removal.

### 1.1. Starting Material 1

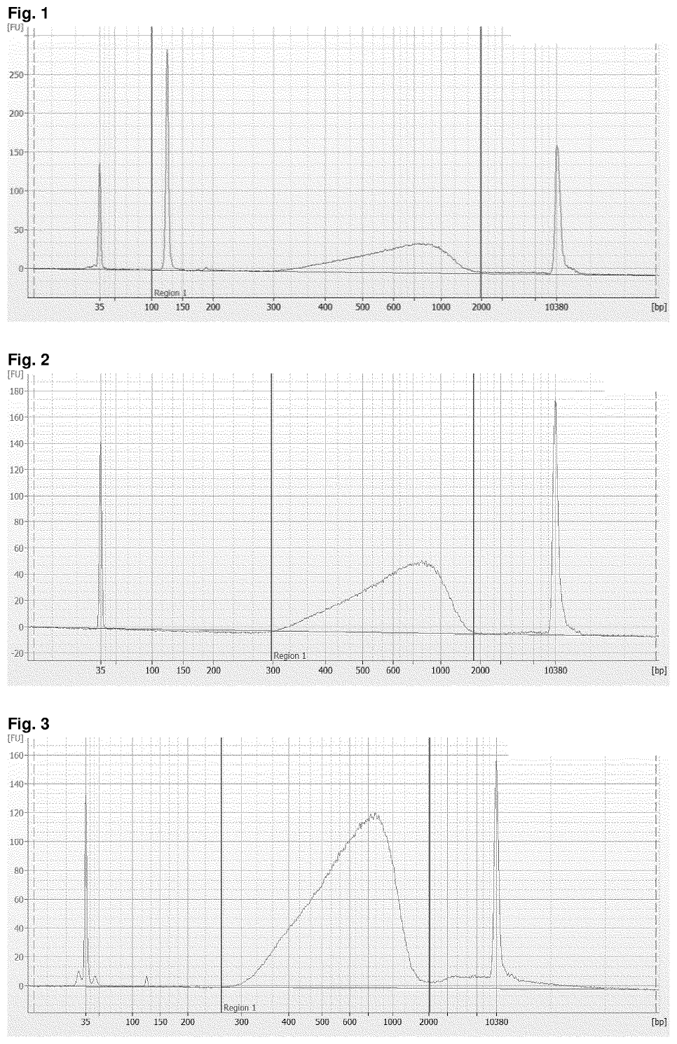

A high quality sequencing library (library A) with a size distribution varying from 300 to 1800 bp with an average fragment size of approx. 700 bp was mixed with an equal volume of a failed library preparation (library B) that comprised vast amounts of adapter dimers (approx. 120-130 bp) in addition to adapter monomers. The resulting DNA containing sample provided a sequencing library with a high amount of sequencing adapter dimers as contaminants (see FIG. 1).

### 1.2. Starting Material 2

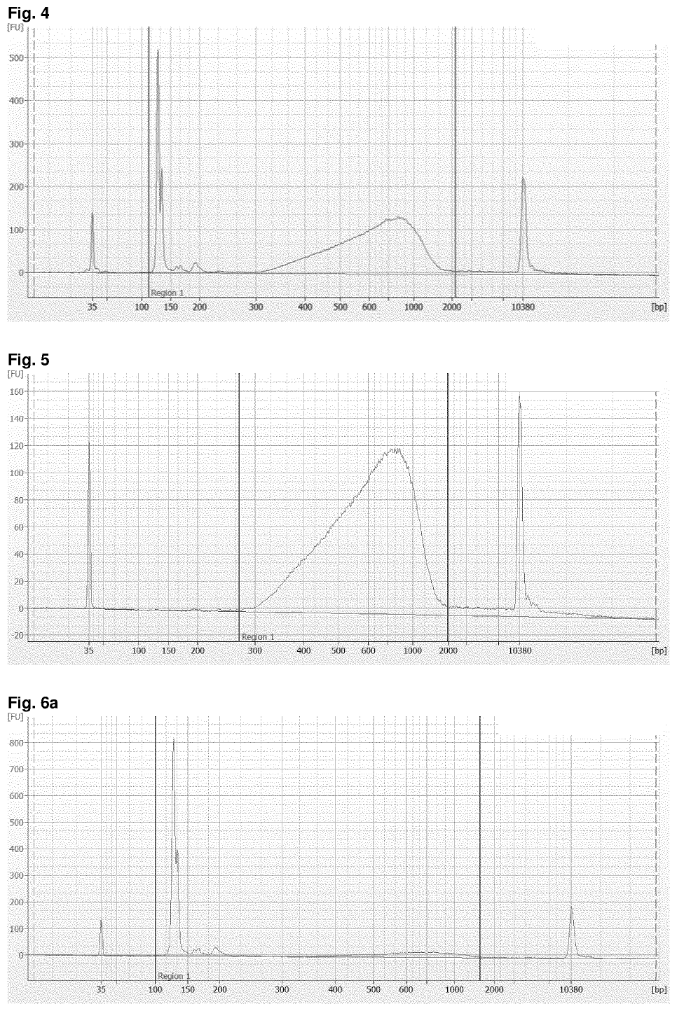

A high quality sequencing library (library A) with a size distribution varying from 300 to 1800 bp was mixed with an equal volume of a failed library preparation (library C). The resulting DNA containing sample provided a sequencing library with a high amount of sequencing adapter dimers (approx. 120-130 bp) and further impurities such as adapter monomers (see FIG. 4).

### 1.3. Starting Material 3

A high quality sequencing library (library D) with a size distribution varying from 221 to 1468 bp, with an average fragment size of approx. 500 bp, was mixed with an equal volume of a failed library preparation (library B) that comprised vast amounts of adapter dimers (approx. 120-130 bp). The resulting DNA containing sample provided a sequencing library with a high amount of sequencing adapter dimers (see FIG. 26).

### 1.4. Starting Material 4

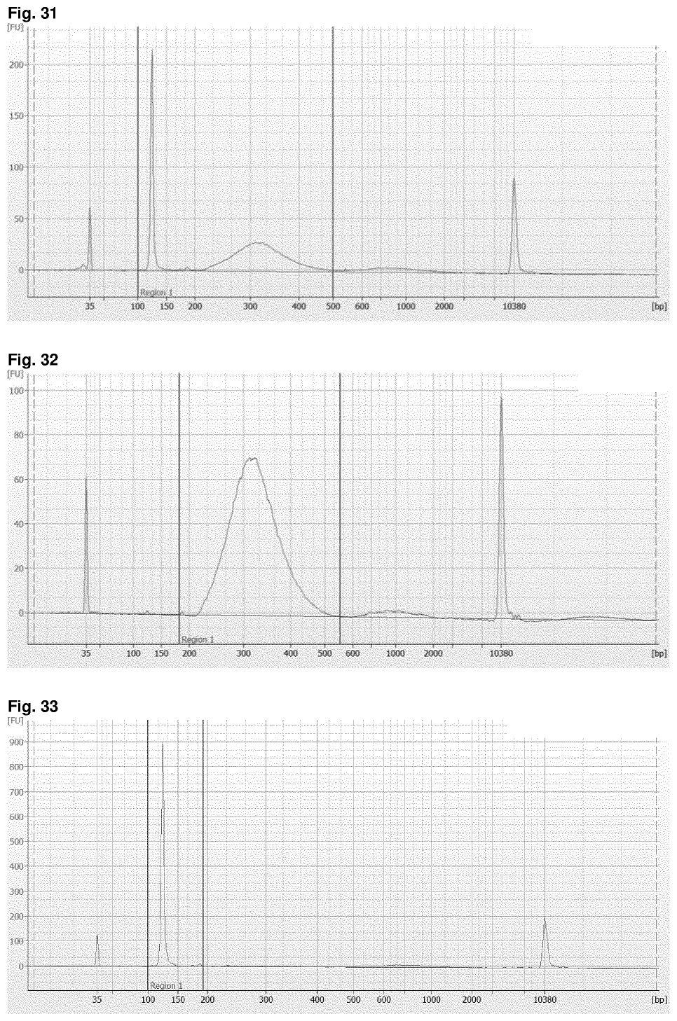

A high quality sequencing library (library E) with a size distribution varying from 230 to 498 bp, with an average fragment size of 330 bp was mixed with a failed library preparation (library B) that comprised vast amounts of adapter dimers (approx. 120-130 bp). The resulting DNA containing sample provided a sequencing library with a high amount of sequencing adapter dimers (see FIG. 31). For preparing starting material 4, the library E and library B was prepared using a 4:3 (v/v) ratio.

### 2. Size Selective DNA Purification

Library DNA fragments were purified from the prepared starting materials with the aim to remove contaminating adapter dimers and other impurities using a prior art reference method or the method according to the present disclosure. The goal of an adapter removal protocol is to specifically remove the adapters (e.g. adapter monomers and adapter dimers) and other impurities from a library while leaving the sequencing library untouched.

If not stated otherwise below, the manual purification methods were performed as follows:

### 2.1. AMPure XP Purification Protocol (According to Beckman Coulter, Calif., USA Manual)—Prior Art Reference

The sequencing adapters were removed from the starting materials with the conventional AMPure XP workflow, consisting of two rounds of size selection:

1. 1× volume (v/v) of the AMPure bead suspension was added to the sample.
2. Sample and beads were mixed through pipetting up and down ten times; the sample was left at RT for 5 minutes.
3. The beads were separated from the supernatant on a magnetic stand, the supernatant was removed.
4. 200 μl of 80% ethanol was added to the sample, sample was washed through pipetting movements.
5. Steps 3 and 4 were repeated
6. The supernatant was removed on a magnetic stand.
7. The beads were air-dried for 10 minutes at RT.
8. 55 μl TE buffer was added to the beads and mixed through pipetting movements for 2 minutes.
9. Steps 1-8 were repeated to ensure complete adapter removal.

This protocol follows the general “bind-wash-elute” principle of sample preparation and cleanup. The initial binding step establishes the adapter removal. The concentration of the AMPure buffer in the binding mixture determines the fragment length of DNA that is able to bind to the carboxylated AMPure beads. The binding conditions are chosen to allow only larger fragments to bind to the beads, whereas smaller fragments (such as adapter impurities) are left in the supernatant. However, since the adapter removal after one round of size-selection purification is still incomplete with this standard protocol, the entire procedure has to be repeated, which is not only time consuming but also costly because the double amount of materials/reagents are used.

### 2.2. Size-Selective Purification Method According to the Present Disclosure

If not indicated otherwise, the manual method according to the present disclosure was performed as follows:

1. 1.1× Volume (v/v) of PEG buffer (20% PEG 8000 (w/v), 2.5 M NaCl, 1 mM EDTA, 0.05% Tween 20 (v/v), 10 mM Tris, 3.36 mM HCl) and 4 μl of carboxylated beads (inter alia M-Beads, MoBiTec, Gottingen, Germany) were added to 1 volume sample (starting material). Here, 1 volume corresponded to 100 μl.
2. Incubation was done for 5 min at 700 rpm on a shaker to ensure DNA binding to the beads. DNA fragments larger than the cut-off (determined by the PEG buffer concentration) bind to the beads, leaving the majority of adapter and adapter dimers in the supernatant under these size selective binding conditions.
3. The supernatant was removed on a magnetic rack.
4. 1.05× volume of PEG buffer (v/v) prediluted in 1 volume TE buffer as specified in the examples below was added to the separated particles and mixed on a shaker at 700 rpm for 1 minute. The chosen conditions favor binding of target DNA fragments, which therefore remain bound to the beads, whereas shorter DNA fragments having a size below the cut-off value are selectively eluted into the supernatant.
5. Steps 3 and 4 were repeated and the supernatant removed completely. Repeating the size-selective elution of smaller DNA fragments improves adapter removal.
6. 200 μl of 80% ethanol were added and mixed in a shaker for 1 mins at 700 rpm.
7. The supernatant was removed.
8. Steps 6 and 7 were repeated.
9. The beads were air-dried for 10 minutes at RT.
10. The DNA was eluted from the beads in 55 μl TE buffer for 3 mins at 700 rpm on a shaker.

### 3. Analytical Methods

The results of the methods were inter alia analyzed by the following methods:

### 3.1. Bioanalyzer (Agilent Genomics)

Various samples were loaded to an Agilent Bioanalyzer (Agilent High Sensitivity DNA Assay, Agilent Technologies) and the resulting electropherograms analysed.

On the electropherograms, marker bands are visible at around 35 and 10380 bp.

### 3.2. PCR Based Assays to Detect Adapter Dimers

To thoroughly assess the purification efficiency of the different methods, the adapter-removed, purified library (eluate) was used as template and amplified in a limited cycle PCR using forward and reverse primers that are essentially identical to the adapter sequences. This PCR assay thereby enriches adapter dimers, if present in the eluate, with great efficiency.

### PCR Assay:

1 ng of purified DNA in a total of 23.5 μl RNase-free water
1.5 μl primer mix (10 μM of each forward and reverse primer)
25 μl of a high fidelity PCR Master Mix 2×
A PCR Run of 12 cycles for amplification of library DNA was performed according to the QIAseq FX DNA Library Kit (96) handbook.

Obtained amplicons were run on a Bioanalyzer. Analysis after the enrichment PCR using a Bioanalyzer allows a thorough assessment of the completeness of the adapter removal. This PCR based assay is very sensitive and amplifies any remaining adapter dimers present in the eluate and thus allows to detect even trace levels of adapter-dimer impurities still present in the eluate after purification that are not visible in an electropherogram of the eluate. This sensitive assay thus enables the determination of removal of adapter dimers was complete or not.

3.3. Column Purification of the Supernatants Harvested from Binding and/or Size-Selective Elution Steps

For assessing the total amount of DNA that was either specifically excluded from binding to the carboxylated beads in the bindings steps of the tested protocols or in the size-selective elution step (c) according to the present invention, the harvested supernatants were column purified using the MinElute® purification kit (QIAGEN) which isolates small and large DNA and accordingly allows to isolate e.g. adapters, adapter dimers as well as library DNA from the harvested supernatants.

### II. Example 1: Size Selective Purification Using the Prior Art Reference Protocol

### 1. Starting Material 1

Starting material 1 (see FIG. 1) was size-selectively purified using the AMPure XP method (gold standard reference), performing two subsequent rounds of adapter removal. The purified, adapter-removed sample (eluate) is shown in FIG. 2.

The purified library (eluate) was furthermore analysed by adapter-enrichment PCR to amplify and thereby detect trace background levels of adapter dimers present in the eluate that were not visible in the electropherogram of the eluate (FIG. 3).

### 2. Starting Material 2

Starting material 2 (see FIG. 4) was size-selectively purified using the AMPure XP method (gold standard reference), performing two subsequent rounds of adapter removal. The purified, adapter-removed sample (eluate) is shown in FIG. 5.

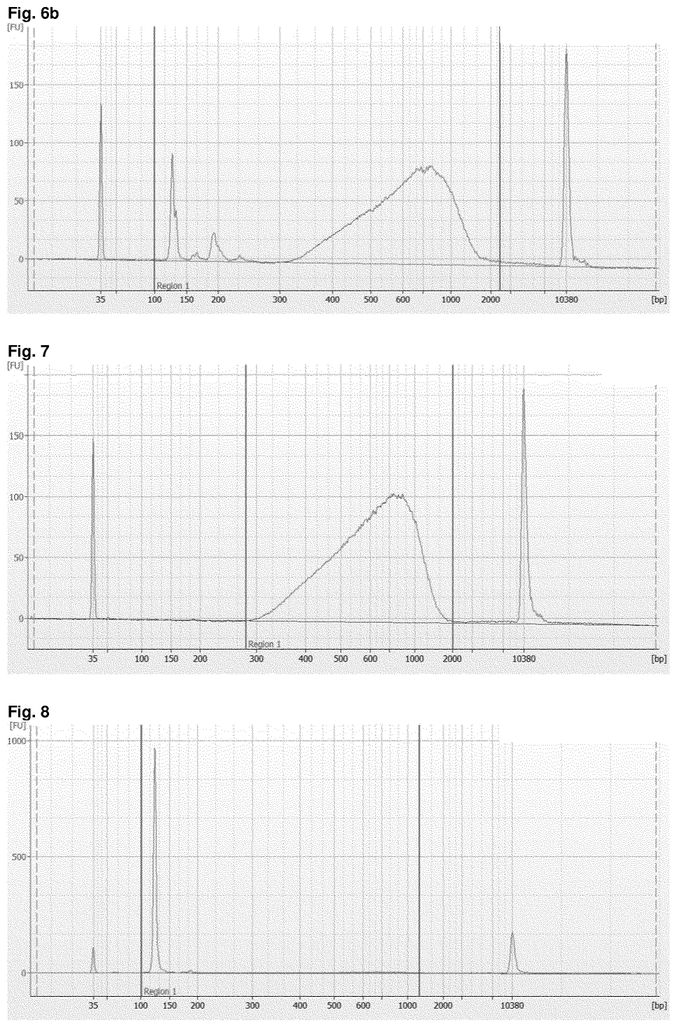

In order to analyze the recovery of target DNA by the prior art method, DNA was furthermore isolated from the supernatant obtained after the 1^{st} (FIG. 6a) and 2^{nd} binding step (FIG. 6b) of each “bind-wash-elute” cycle, using a spin column procedure and the obtained eluate analyzed on a bioanalyzer.

### DISCUSSION

The data shows that the prior art AMPure XP adapter removal protocol efficiently removes impurities such as adapter dimers from the starting material, if performing two cycles of size selection. The electropherograms of the obtained eluates show that the sequencing adapters present in the starting material were virtually removed, while the sequencing library itself (comprising the target DNA molecules) shows in the eluate the same size distribution as before. Some library DNA was lost in particular during the second binding cycle.

Following the adapter-enrichment PCR, a small peak at a size of 120 bp becomes visible on the electropherogram. This indicates that although the adapters were removed from the sample with the industry standard AMPure XP protocol, minor amounts of residual adapter dimers remained in the eluate and were detectable by PCR. Yet, this portion of remaining adapters was very small and thus unlikely to affect any downstream sequencing applications.

In addition, it is noted that the used starting materials were highly contaminated with adapter-dimers.

In sum, while the prior art standard protocol efficiently removes adapter-dimers and other small impurities, it has the drawback that it is time and material consuming because two complete cycles of size selection (“bind-wash-elute”) must be performed to achieve this adapter-removal efficiency. These are considerable disadvantages considering that such adapter-removal purifications are performed for high-throughput applications for NGS applications.

### III. Example 2: Size Selective Purification Using the Method According to the Present Disclosure

After having established in example 1 that the adapter removal with the industry standard AMPure XP workflow method is essentially complete and a PCR enrichment of adapter dimers is hardly possible after their removal from the sample, the same experiment was repeated using the more time and cost-efficient size-selective purification protocol according to the present disclosure. As in the reference protocol, a first size selection is achieved in binding step (a), in which target DNA fragments larger than a certain cut-off (determined by the polyethylene glycol concentration) bind to the beads, leaving the majority of adapter and adapter dimers in the supernatant. After binding to the magnetic particles, the solid phase is separated from the remaining sample, here by removing the supernatant. A further level of size selection is then performed through selective elution of bound non-target DNA fragments. The polyethylene concentration during size selective elution of non-target DNA fragments is chosen so it favors the binding of the longer, target DNA fragments to the beads, whereas the shorter non-target DNA fragments are eluted. If needed, this can be followed by a second size selective elution step to ensure complete adapter-fragment removal.

### 1. Starting Material 1

In this example, the concentration of the PEG buffer was reduced from 1.1× PEG-buffer during binding step (a) to 1.05× PEG buffer in the size selective elution step (c). The purified library (eluate) is shown in FIG. 7.

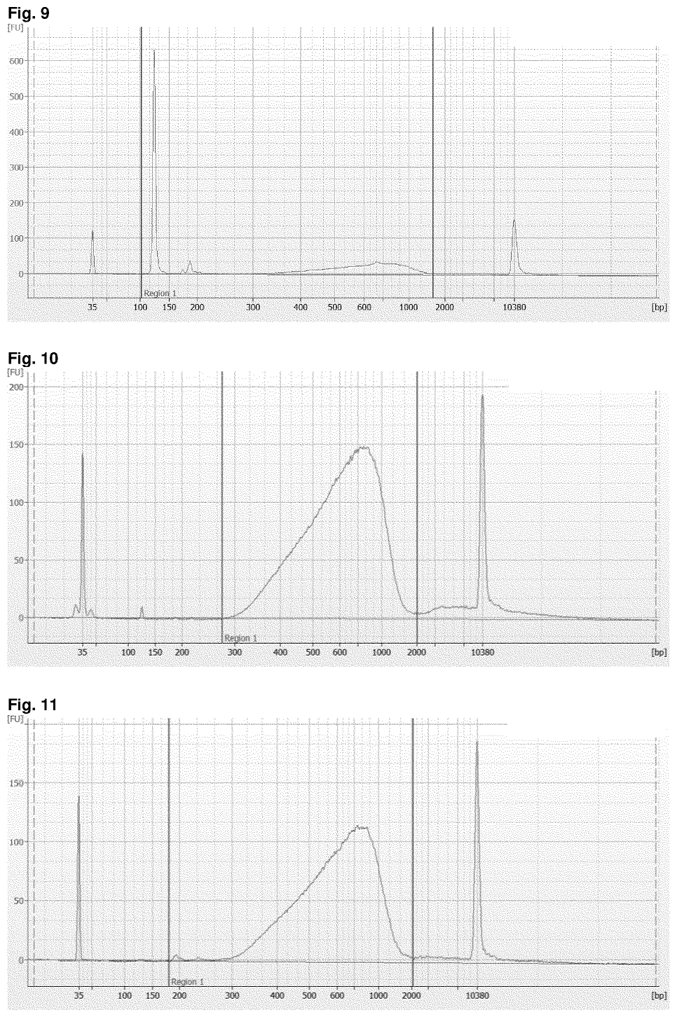

In order to analyze the recovery of the target DNA by the method according to the present disclosure, DNA was furthermore isolated from the supernatant obtained after binding step (a) (FIG. 8) as well as the supernatant of first size-selective elution step (c) (FIG. 9) using a spin column procedure and the eluates obtained from the spin-column clean-up were analyzed on a bioanalyzer.

Subsequently, the adapter-removed, purified library (eluate) obtained with the present method was subjected to the adapter-enrichment PCR to analyze the purified eluates for trace background levels of adapter dimers (FIG. 10) that could not be observed in the electropherograms without amplification in the purified samples.

### 2. Starting Material 2

In this example, the concentration of the PEG buffer was reduced from 1.1× PEG-buffer during binding step (a) to 1.05× PEG buffer in the size selective elution step (c). The purified library (eluate) is shown in FIG. 11.

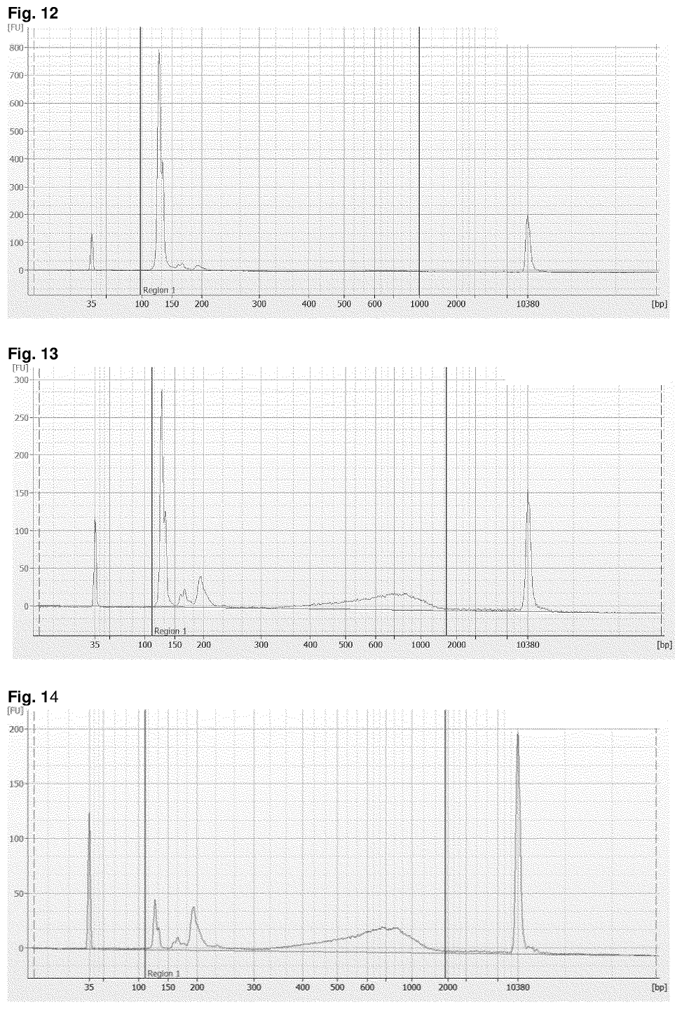

In order to analyze the recovery of the target DNA by the method according to the present disclosure, DNA was furthermore isolated from the supernatant obtained after the binding step (FIG. 12) as well as the supernatant of the first (FIG. 13) and second (FIG. 14) size-selective elution steps using a spin column procedure and the obtained eluate analyzed on a bioanalyzer.

### DISCUSSION

The electropherograms of the obtained adapter-removed eluates show that the adapter-dimers present in the starting material were efficiently removed with the method according to the invention, while the sequencing library itself (comprising the longer target DNA molecules) showed in the eluate the same size distribution as in the starting material. Virtually no adapter dimers were present in the obtained eluates. Following the adapter-enrichment PCR, again only a small peak at a size of 120 bp becomes visible the electropherogram. Hence, as in the (more time and cost-intensive) industry standard, only a very small amount of residual adapter dimers remained in the sample (eluate) as detected by PCR.

Therefore, performing after separation of the solid phase from the binding mixture a size-selective elution step (c) by contacting the solid phase with the bound nucleic acids with a reagent composition that preferably comprises either the same or a lower poly(alkylene) oxide polymer concentration than is used in the binding mixture specifically and efficiently removes non-target DNA fragments that are smaller than the target DNA library from the beads thereby significantly improving the purification results.

Example 2 thus demonstrates that the size-selective purification method of the invention is at least as efficient as the industry standard in achieving adapter removal, while, however, considerably reducing the handling time and effort needed and thereby accelerating the speed of sample preparation. These findings underline the potential and advantages of the method of the invention, which is less time consuming, requires less hands-on time compared to the conventional method (AMPure XP protocol), uses less consumables and beads and is therefore also more cost-efficient. Altogether, the method according to the present disclosure is faster and cheaper than the conventional workflow, while providing high quality results.

Moreover, without the need to elute and re-bind the target DNA fragments half way through the overall protocol (as is required in the prior art protocol), the method according to the present disclosure can advantageously be automated on simple processing devices without the need for complex liquid handling devices such as pipetting machines. Size-selective DNA isolation can be conducted by simple automation, which does not necessarily need to comprise a pipetting unit.

### IV. Example 3: Automation of the Method According to the Present Disclosure

The method according to the present disclosure can be automated and performed on simple devices without the need for liquid handling. The manual workflow was therefore adjusted for automation as shown in Table 1.

An example of an automated protocol is depicted below in Table 1. The individual cavities of the plates were loaded with the solutions as described in Table 1 which shows the loading scheme for an automated adapter-removal protocol:

TABLE 1

| Automated | | | | | | |
|---|---|---|---|---|---|---|
| instrument - | | | | | | |
| sample plate | Position 1 | Position 2 | Position 3 | Position 4 | Position 5 | Position 6 |
| Buffer | Sample (binding | 1.05 × PEG | 1.05 × PEG | 80% | 80% | TE |
| | mixture comprising | | | Ethanol | Ethanol | |
| | magnetic particles | | | | | |
| | with bound nucleic | | | | | |
| | acids) | | | | | |
| Amount (μl) | 525 | 225 | 225 | 225 | 225 | 75 |
| Drops | Pick-up | 15 | 15 | 15 | 15 | 25 |

In brief, a sample comprising the binding mixture which comprises the carboxylated beads was prepared and transferred to a magnetic bead extraction platform (e.g. Gilson Extractman®; Gilson Inc., Middleton, US/Salus Discovery). DNA comprised in the starting material was size-selectively bound to carboxylated magnetic beads in presence of the binding buffer (1.1× PEG buffer) to establish binding conditions on a shaker before placing the binding mixture into the first cavity of an extraction platform plate (position 1). Magnetic particles with bound DNA were separated from the sample solution by application of a magnetic field. Specifically, the magnetic particles were pulled out of the solution. Bound beads were then transferred into the selective elution solution (1.05× PEG buffer) by release of the particles into the selective elution solution in a sample well (position 2), agitation and re-attracting/collecting the magnetic particles with the bound DNA. In most steps, the cycle of collecting the particles and releasing them was repeated in order to impose some mild mechanical force onto the particles to mix the samples during cleanup and elution steps. The number of “Drops” of the magnetic beads in each step is shown in the table. Optionally, the selective elution step (c) can be repeated to improve adapter removal by transferring the magnetic particles with bound DNA again into the selective elution solution (1.05× PEG buffer) (position 3). Subsequently, two wash steps with 80% ethanol were performed. The ethanol removal step of the manual protocol was omitted. Release/Elution of nucleic acids was carried out by removing/relocating the magnetic field, such that the magnetic beads with nucleic acid bound thereto dropped into the elution solution (TE-buffer). After elution, the magnetic particles can be removed from the eluate or the eluate can be removed as supernatant, while attracting the magnetic particles via a magnet. The overall procedure thus resembles the manual protocol with minor adaptations to the automation platform.

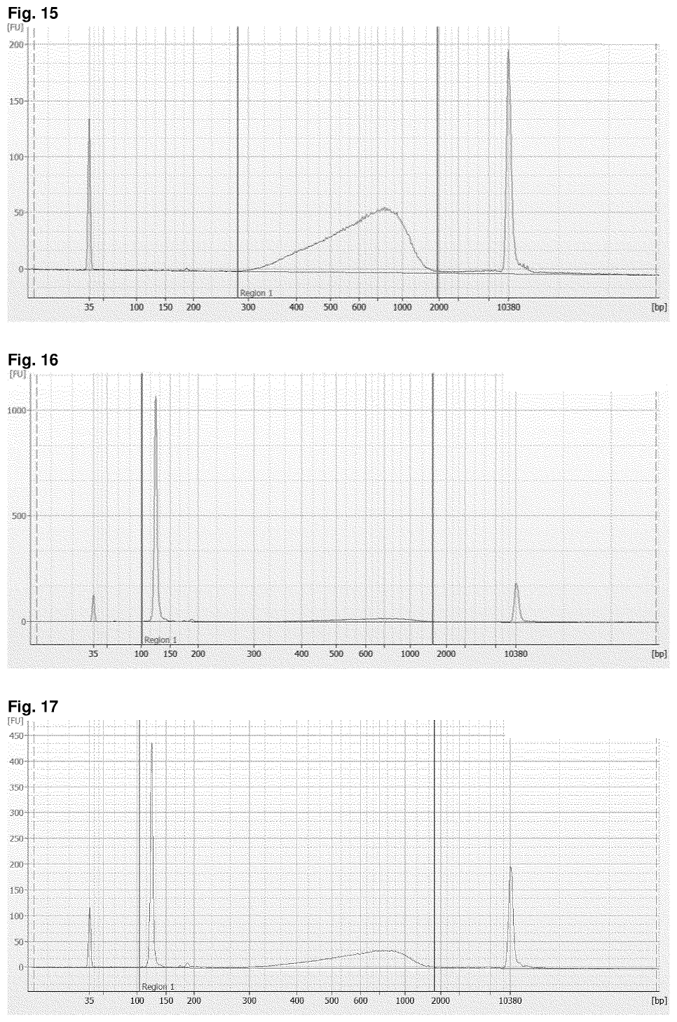

The eluates of starting materials 1 and 2 that were purified using the automated method according to the invention were subjected to analysis on a bioanalyzer (FIGS. 15 and 20).

### 1. Starting Material 1

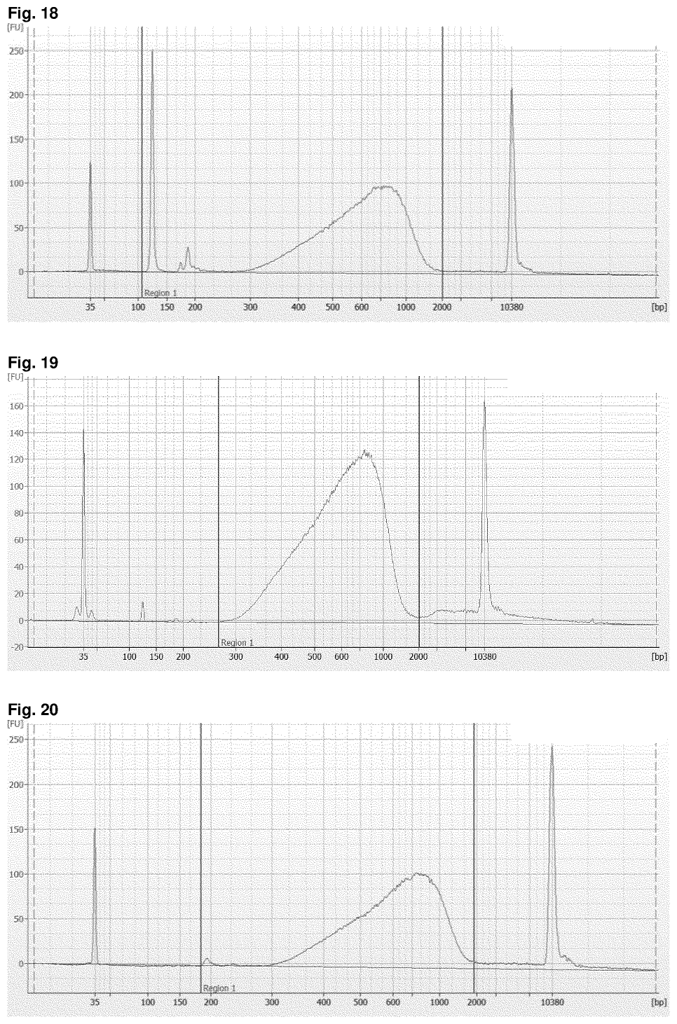

The recovery of library DNA and elution of impurities during the individual cleanup steps (purified eluate, see FIG. 15; binding step, see FIG. 16; 1^{st} size-selective elution step, see FIG. 17; 2^{nd} size-selective elution step, see FIG. 18) was analyzed using spin-column cleanup of the supernatants followed by DNA measurements using the Agilent Bioanalyzer.

Purified eluates of starting material 1 were moreover used as a template for adapter-enrichment PCR to investigate the presence of trace amounts of remaining adapter dimers (FIG. 19).

### 2. Starting Material 2

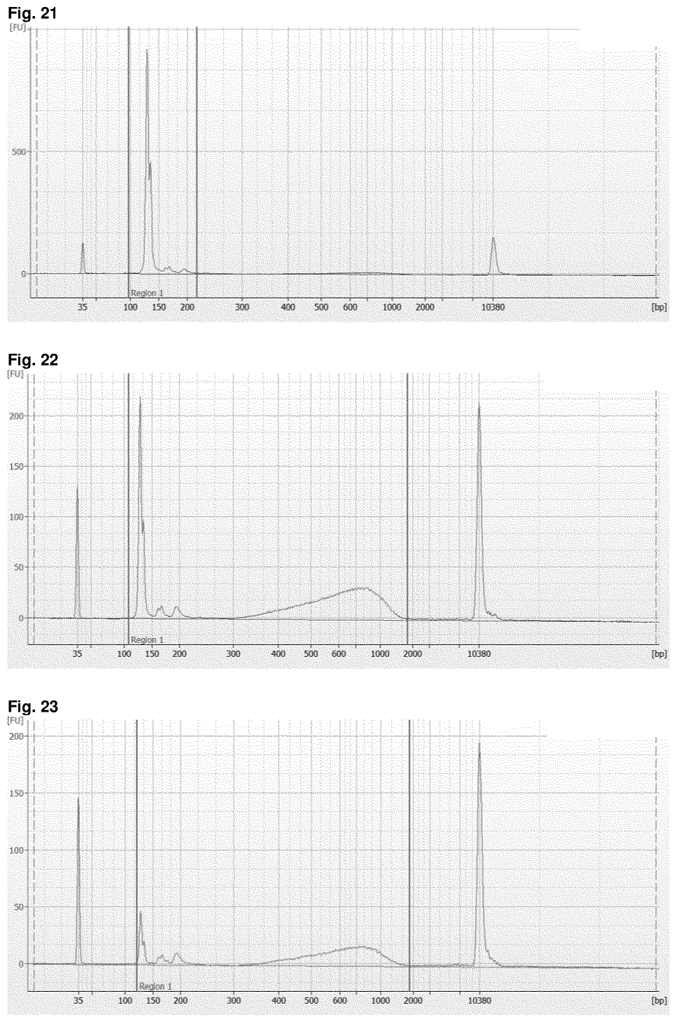

The recovery of library DNA and elution of impurities during the individual cleanup steps (purified eluate, see FIG. 20; binding step, see FIG. 21; 1^{st} size-selective elution step, see FIG. 22; 2^{nd} size-selective elution step, see FIG. 23) was analyzed using spin-column cleanup of the supernatants followed by DNA measurements using the Agilent Bioanalyzer.

### DISCUSSION

The electropherograms show highly successful adapter removal when using the automated method according to the invention. The obtained purity is very high and comparable to the results of the manual procedure or prior art protocol. These findings corroborate the notion that the method of the invention can be easily automated without the need for complex pipetting robots for the individual purification steps. The performed automated runs took under 10 minutes from start to finish and significantly reduced the overall time required for the procedure.

Similar to the manual protocol tested, detailed analysis by adapter-enrichment PCR showed the presence of only minute levels of adapter dimers after purification. Performing a size selective elution step after the size-selective binding step efficiently and selectively removes remaining adapter dimers and other DNA impurities from the beads. Performing a second size-selective elution step may further increase the purity. This can be advantageous, if the starting material is highly contaminated with small DNA fragments (as in the present examples) to further improve the purity.

### V. Example 4: Recovery of Library Sample in the Different Methods

The DNA yield recovered by the different methods was evaluated.

### 1. Starting Material 1

The following methods were compared, as summarized in Table 2:

TABLE 2

| | AMPure | Invention, | Invention, |
|---|---|---|---|
| Sample Name | (reference) | manual | automated |
| Purification | Method 2.1. | Method 2.2. | Method 2.2. |
| method | | manual | automated |
| | | protocol | protocol |
| Beads used | AMPure | M-Beads | M-Beads |
| | beads | | |

In the methods according to the invention, the concentration of the PEG buffer was reduced from 1.1× PEG-buffer in the binding step to 1.05× PEG buffer in size-selective elution step (c).

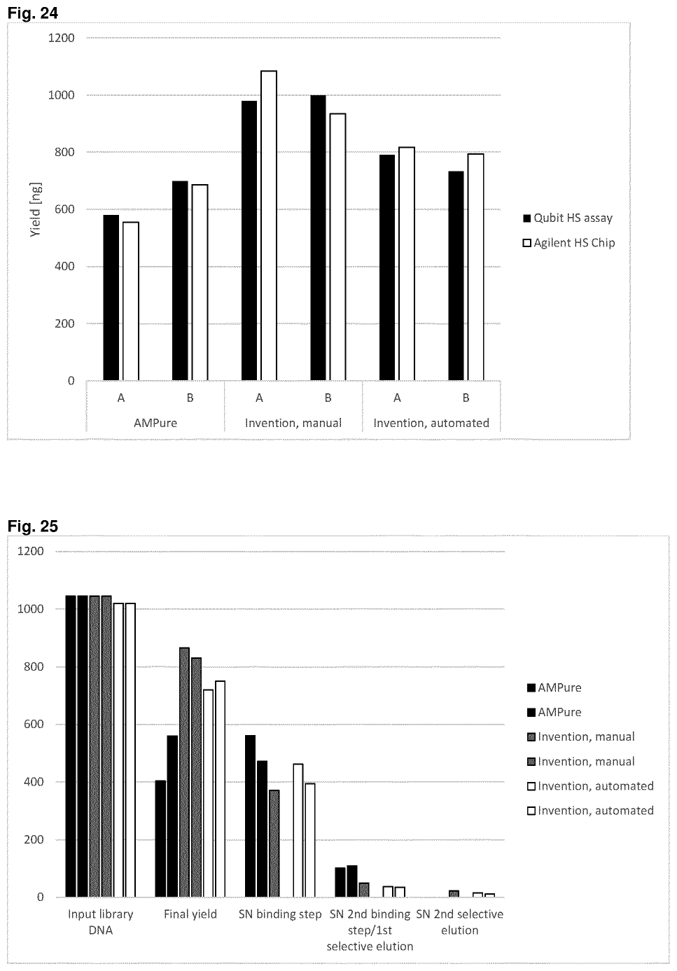

The DNA yield in the adapter-removed, purified eluates is shown in FIG. 24. Concentrations were determined using fluorometric means (Qubit) as well as a bioanalyzer. Duplicate sample preparation (A, B) and analysis is shown.

### 2. Starting Material 2

For starting material 2, the manual and automated methods according to the invention were compared to the prior art method (AMPure). In both protocols according to the invention, was the concentration of the PEG buffer reduced from 1.1× PEG-buffer in the binding step to 1.05× PEG buffer prediluted in 1 volume TE buffer in size-selective elution step (c). Total DNA subjected to the different purification protocols as well as the finally recovered DNA library yield in the purified eluates is shown in FIG. 25. Potential DNA losses during the individual cleanup steps were furthermore analyzed by spin-column cleanup of the supernatants followed by DNA measurements using the Agilent Bioanalyzer.

### DISCUSSION

The same DNA amounts of starting material were subjected to the different purification workflows. The majority of DNA fragments smaller than the cut-off, such as adapter dimers, are efficiently removed during the initial binding step because they are not bound to the beads (see e.g. FIG. 25). However, some of these smaller non-target DNA fragments still bind to the solid phase and are therefore also separated together with the beads from the binding mixture. These co-bound impurities were subsequently removed during the subsequent rounds of cleanup (second cycle of the AMPure protocol) or the size selective elution step according to the present invention which is more time- and cost efficient than the prior art reference method.

The method according to the present disclosure moreover showed higher DNA yields in the obtained eluates than the established AMPure bead procedure. This was even the case when the workflow was automated on a simple bead handling device (see FIGS. 24 and 25). The AMPure protocol loses DNA library in particular in the second “bind-wash-elute” cycle (see also Example 1). Loss of target DNA was advantageously reduced when using the method according to the present invention.

### VI. Example 5: pH and Library Size Variations Using the Manual Protocol of the Invention

To further assess the applicability of the present method in cases in which the size difference between the largest impurity and smallest amplicon of the library was reduced, target DNA (library) was isolated from starting material 3 (FIG. 26) and starting material 4 (FIG. 31) using the method of the invention.

Furthermore, the pH of the PEG-containing buffers was altered. PEG-containing buffers used in step (a) and (c) with a pH of 4.6 were compared to corresponding buffers with a pH of 8.2. A concentration of 1.05× PEG-containing buffer was used for the size-selective elution step (c) for both starting materials 3 and 4.

### 1. Starting Material 3

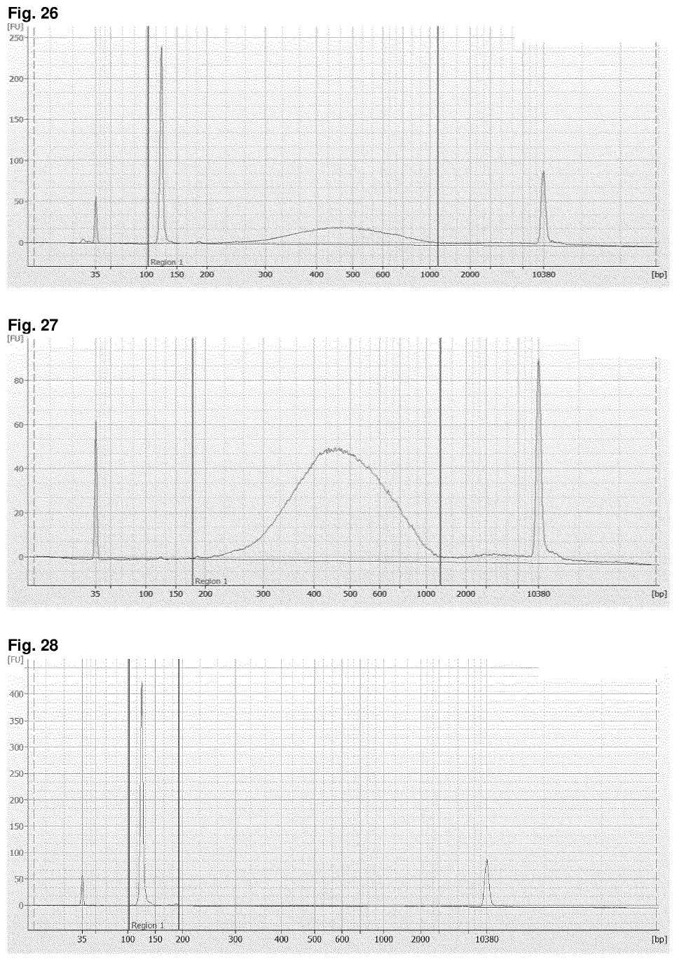

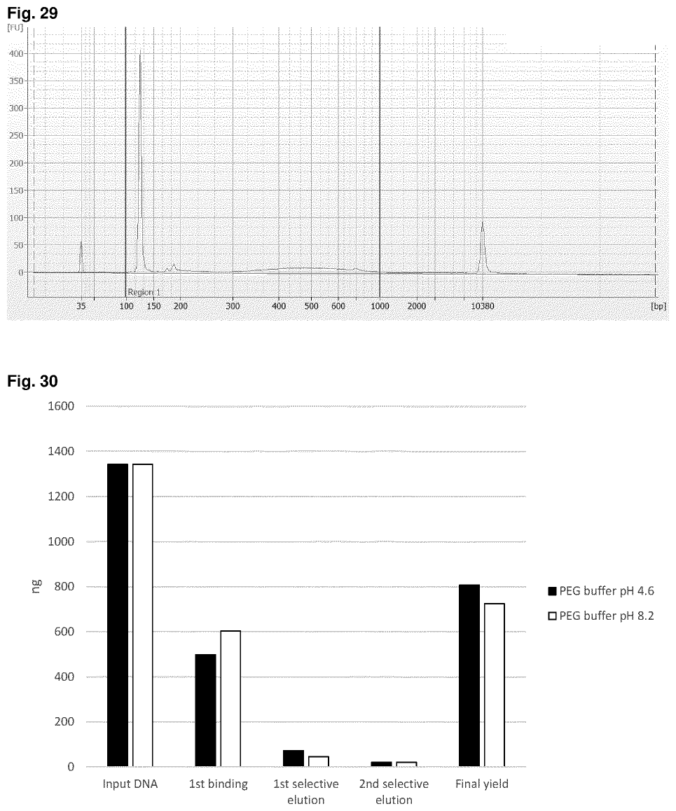

Samples of starting material 3 (FIG. 26) were purified manually using the method according to the invention. The performance of the method of the invention was assessed using starting materials containing smaller fragment sizes, i.e. reducing the size difference between the impurities and the smallest fragment of interest. In addition, the effect of altering the pH of the PEG-containing buffers was analysed. The recovered DNA in the eluate (FIG. 27), as well as in the supernatant of step (a) (FIG. 28) or the supernatant provided in step (c) (FIG. 29) was evaluated. Furthermore, the DNA yield obtained with the buffers having different pH values was analyzed (FIG. 30).

### 2. Starting Material 4

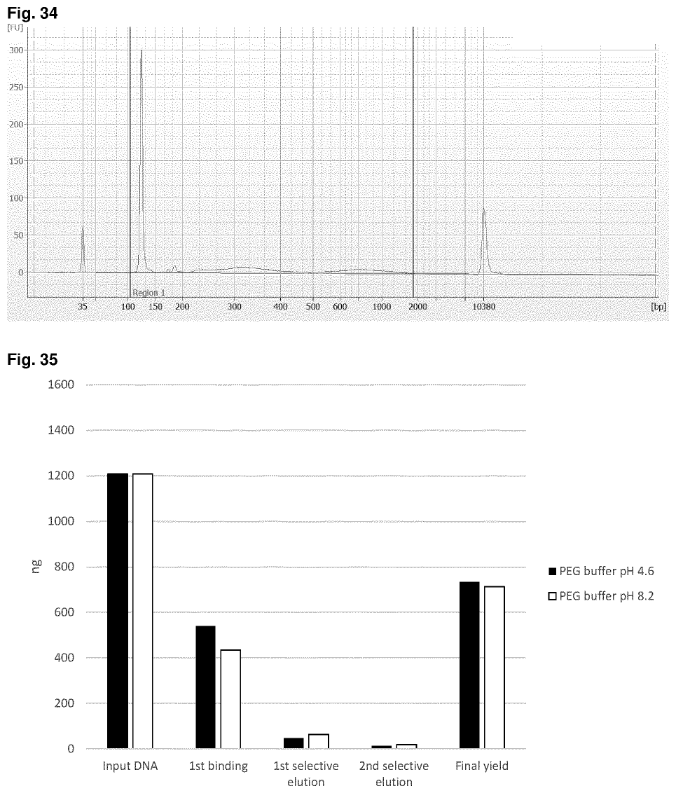

The average size of the DNA fragments contained in the library of starting material 4 was reduced compared to starting material 3. Sample of starting material 4 (FIG. 31) was purified manually using the method according to the invention and the obtained eluate was analysed (FIG. 32). The DNA excluded from binding to the beads during binding step (a) (FIG. 33) or selectively eluted from the beads during size-selective elution step (c) (FIG. 34) was analyzed by spin-column cleanup of the supernatants followed by DNA measurements of the spin-column purified DNA using a bioanalyzer (High Sensitivity DNA Analysis Kit, Agilent Genomics).

In order to assess the quality and efficiency of the method according to the invention using starting materials containing smaller amplicon sizes, i.e. reducing the size difference between the impurities and the smallest amplicon, as well as the effect of altering the pH of the PEG-containing buffers (pH 4.6 and pH 8.2), the amount of recovered DNA in the eluate purified from starting material 4 as well as the DNA not bound to the beads in binding step (a) or selectively eluted from the beads in size-selective elution step (c) was evaluated and compared (FIG. 35).

### DISCUSSION

The same DNA amounts of starting material 3 (FIG. 26) and starting material 4 (FIG. 31) containing on average smaller fragment sizes than starting materials 1 and 2 were subjected to the purification method according to the invention. As seen before for starting materials 1 and 2, the majority of non-target DNA fragments such as adapter dimers do not bind to the beads in binding step (a) and therefore remain in the supernatant and are thereby efficiently depleted. The library DNA is, however, effectively bound. This provides a first level of purification and non-target DNA depletion. Co-bound non-target DNA fragments such as adapter dimers are furthermore selectively eluted from the solid phase in subsequent size-selective elution step (c), which optionally may be performed twice to improve the results. The provided eluates are very pure due to the efficient removal of non-target DNA. The final target DNA yield in the eluates is high, demonstrating that a loss of target DNA fragments (library) during the processing steps can be kept to a minimum in the method of the invention. Similar results were observed at different pH values.

Accordingly, the method of the invention allows to provide a highly pure target DNA, such as DNA libraries, with high yield in a time- and cost efficient manner. The method can be used over a broad pH range, is reliable, fast and cost-efficient.

### VII. Example 6: Variation of Peg Concentration and Molecular Weight

The effectiveness of the size-selection procedure via selective-elution was shown in the previous experiments. Example 6 focusses on the composition of the PEG-buffer by varying (1) the PEG concentration and (2) the molecular weight of the used PEG molecules.

### Preparation of Nucleic Acid Containing Sample as Starting Material

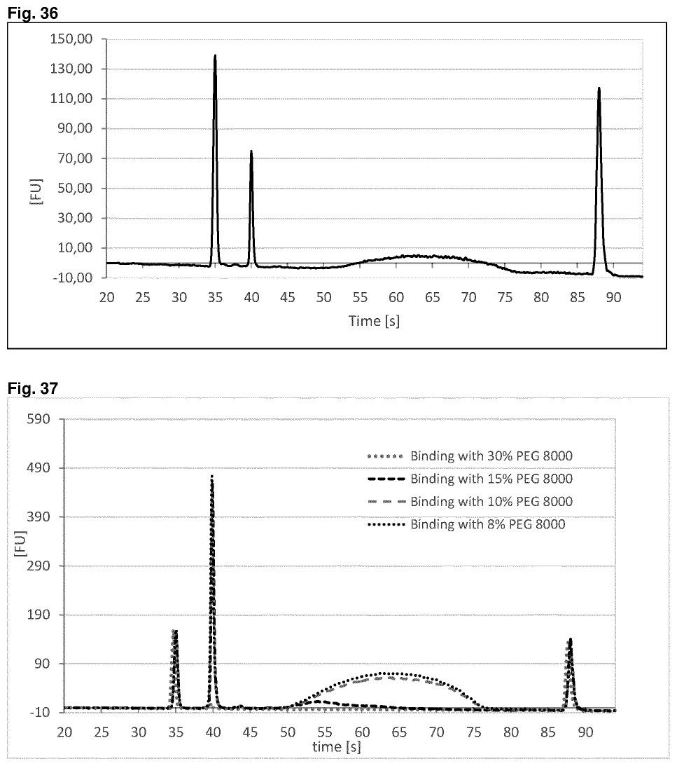

As nucleic acid containing sample, an adapter-dimer contaminated sequencing library was prepared to assess the effectiveness of adapter removal. A high quality sequencing library (library A) with a size distribution varying from 300 to 1800 bp with an average fragment size of approx. 700 bp was mixed with an equal volume of a failed library preparation (library B) that comprised vast amounts of adapter dimers (approx. 120-130 bp) in addition to adapter monomers. The resulting DNA containing sample provided a sequencing library (starting material) with a high amount of sequencing adapter dimers as contaminants (see FIG. 36).

### Experimental Procedure

Using the method according to the present disclosure, library DNA fragments were purified from the prepared starting materials with the aim to remove contaminating adapter dimers and other impurities from the sequencing library.

### Protocol

The following protocol was followed in Example 6:
1. 1.1× Volume (v/v) of PEG-buffer (see below) and 4 μl of carboxylated beads (inter alia M-Beads, MoBiTec, Gottingen, Germany) were added to 1 volume sample (starting material).
2. Incubation was done for 5 min at 1400 rpm on a shaker to ensure DNA binding to the beads.
3. The supernatant was removed on a magnetic rack.
4. PEG-buffer (v/v) was prediluted with TE buffer as specified in the examples below and added to the separated particles and mixed on a shaker at 900 rpm for 1 minute. Different dilutions were analysed as specified below for binding of target DNA fragments, which therefore remain bound to the beads. Undesired shorter DNA fragments having a size below the cut-off value are selectively eluted from the particles into the supernatant.
5. Steps 3 and 4 were repeated and the supernatant removed completely. Repeating the size-selective elution of smaller DNA fragments improves adapter removal and can be optional.
6. 200 μl of 80% ethanol were added and mixed in a shaker for 1 minute at 900 rpm.
7. The supernatant was removed.
8. Steps 6 and 7 were repeated.
9. The beads were air-dried for 15 minutes at RT.
10. The DNA was eluted from the beads in prewarmed (65° C.) 50 μl AE buffer (QIAGEN) for 3 mins at 1400 rpm on a shaker.

### Analytical Methods and Sample Preparation for Analysis

The results of the methods were inter alia analyzed by the following methods:
Various samples were loaded to an Agilent Bioanalyzer (Agilent DNA 7500 Kit, Agilent Technologies) and the resulting electropherograms analyzed. On the electropherograms, marker bands are visible at around 35 and 10380 bp, as is derivable from the seconds indicated on the x-axis.
Analyses were also performed using the Qubit High Sense dsDNA Kit (Invitrogen by Thermo Fisher).
A column purification of the supernatants harvested from the binding steps and/or size-selective elution steps was performed for assessing the total amount of DNA that was either specifically excluded from binding to the carboxylated beads in the bindings step (a) or during the size-selective elution (c) according to the present invention. The harvested supernatants were column purified using the MinElute® purification kit (QIAGEN) which isolates small and large DNA and accordingly allows to isolate e.g. adapters, adapter dimers as well as library DNA from the harvested supernatants.

### Example 6.1: Use of PEG 8000 in Different Concentrations

In this experiment the effect of different PEG concentrations in the PEG-buffer on the effectiveness of the size-selection procedure was analysed. The following concentrations of PEG were used in the PEG-buffer: 8% PEG 8000 (w/v), 10% PEG 8000 (w/v), 15% PEG 8000 (w/v) and 30% PEG 8000 (w/v). In addition to PEG 8000 in varying concentrations, all PEG-buffers contained the following components 2.5 M NaCl, 1 mM EDTA, 0.05% Tween 20 (v/v), 10 mM Tris, 3.36 mM HCl.

a) Binding of Starting Material to the Carboxylated Beads Using 1.1× Volume of PEG 8000 Buffer with Different PEG Concentrations

An increase in PEG concentration during the initial binding step will not only result in binding of the target DNA but will also facilitate the binding of smaller DNA fragments out of the sample onto magnetic particles. The standard binding dilution of the PEG-buffer used in previous examples, namely adding 1.1× volume of PEG-buffer to 1 volume sample (staring material), was used to bind the library DNA to the magnetic particles. The remaining unbound DNA still present in the supernatant of the binding step was purified using spin-columns and analyzed using a bioanalyzer. The resulting electropherogram is depicted in FIG. 37.

The presence of PEG 8000 in high concentrations facilitates the binding of DNA to the magnetic particles as shown in FIG. 37. Using a PEG-buffer with a concentration of 30% (w/v) bound the entire DNA sample including the adapter dimers to the magnetic particles. Decreasing the PEG 8000 concentration to 15% (w/v) in the PEG-buffer weakened the DNA binding conditions, so that the adapters remained unbound and were thereby already depleted during the binding step. However, in this experimental set-up also some library fragments remained in the supernatant. A further reduction of the PEG 8000 concentration in the PEG-buffer resulted in further weakening of the binding conditions leading to an increased loss of the used library during the binding step because not all target DNA was effectively captured. Hence, if a low PEG concentration is used in the binding buffer, this would need to be balanced by a higher volume of PEG buffer during binding, to increase the PEG concentration in the binding mixture. In conclusion, the PEG concentration influences the size-selection during the initial binding step.

### b) Size-Selective Elution Using PEG-Buffer in Varying Concentrations

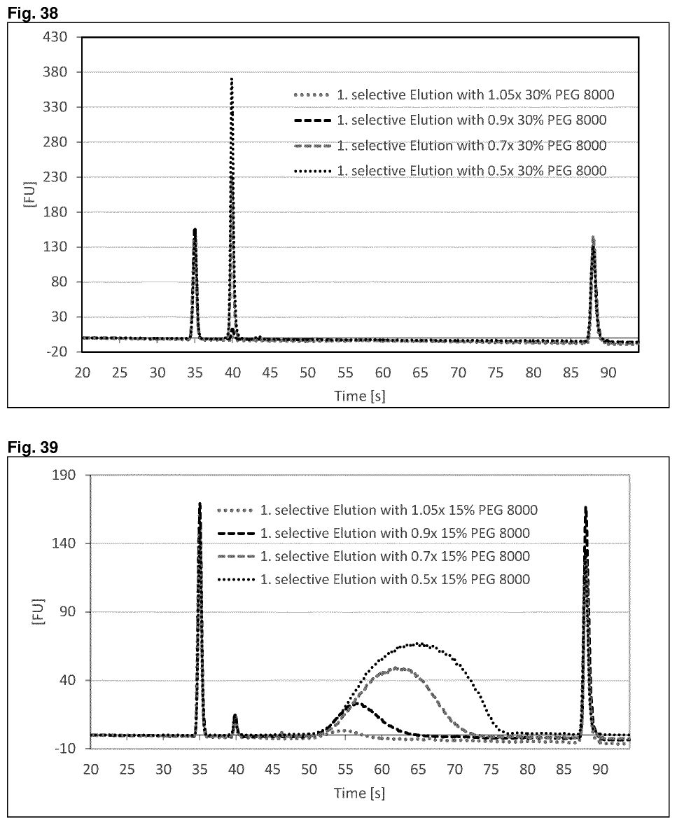

After binding the starting material (library) using buffers with varying PEG concentrations (see Example 6.1. a)), the magnetic particles with the bound DNA were separated and subjected to the selective-elution procedure (step (c) of the present method). Since using a PEG-buffer (reagent) with highly concentrated PEG will influence the PEG concentration during the selective elution for a given dilution factor, multiple dilutions were analyzed to assess optimal conditions for each PEG-buffer (see FIG. 38). DNA that was eluted into the supernatant during the first selective-elution step of the protocol was spin-column purified from the supernatant and the purified DNA was analyzed using a bioanalyzer. The resulting electropherogram is shown in FIG. 38.

FIG. 38 shows an overlay of electropherograms of the spin column purified DNA from the supernatant of the 1^{st} size selective elution steps with different mixing ratios of 30% PEG 8000 (w/v) and Buffer TE as indicated in the legend (e.g. 1.05× volume PEG-buffer+1 volume TE buffer; 0.9× volume PEG buffer+1 volume TE buffer etc.). Since working with 30% PEG in the PEG buffer retrieved all DNA including the adapter dimers from the starting sample during the binding step, it was expected that lower PEG concentrations and thus stronger dilutions of the 30% PEG-buffer were required in order to thoroughly elute the adapters from the beads during the size-selective elution step. In the analyzed set-up, working with a relatively low dilution factor of 1.05× volume (PEG-buffer to TE) the starting material remained bound to the carboxylated beads and no substantial elution of the adapter dimers took place. Lowering the mixing ratio down to 0.7× volumes improved elution of the adapter dimers from the beads. A complete size selective elution was achieved with a mixing ratio of 0.5× volumes. Under these conditions the library DNA remained bound to the carboxylated beads, while the adapters were effectively eluted into the buffer, respectively the supernatant. Hence, when the starting PEG concentration in the PEG buffer is high, higher dilutions of the PEG-buffer are necessary to achieve an efficient size-selective elution. In comparison, lower PEG concentrations in the PEG binding buffer (reagent) would require less dilution of the PEG-buffer for the selective-elution process for two reasons: First, less adapters are bound to the magnetic beads during the binding step (because of the size-selective binding conditions induced by the lower PEG concentration in the binding mixture) and thus need to be removed during size selective elution/washing. Second, because the concentration in the PEG-buffer (reagent) is already low, lower dilutions are needed to achieve eluting conditions for the non-target DNA during the selective elution step. This was tested using a PEG-buffer containing 15% PEG (w/v) for binding and as reagent for preparing reagent composition (c) by dilution. FIG. 39 shows the results of the selective elution at multiple dilution factors when using a PEG-buffer containing 15% PEG.

FIG. 39 substantiates the finding of how the PEG concentration influences the selective elution procedure. Working with high PEG 8000 concentrations by using a dilution factor of 1.05 volumes (PEG-buffer to TE) for the size-selective elution, unwanted adapter dimers were removed from the beads, while the bound library DNA predominantly remained bound to the beads. Increasing the dilution factor and thus lowering the PEG 8000 concentration during the size-selective elution step resulted in increased library loss. However, it is noted that in the chosen experimental set-up, the final PEG 8000 concentrations of the dilutions are depending on the PEG concentration in the PEG-buffer that was used for binding. It is stressed that the end concentration of the PEG in the selective elution buffer, respectively the reagent composition, that is contacted with the beads comprising the bound DNA is of importance, rather than the dilution factor during this step. As disclosed herein, it is e.g. also within the scope of the present invention to use different PEG buffers/reagents for the binding step and the size-selective elution step so that it is not required to dilute a (single) PEG buffer (e.g. binding reagent) to adjust the conditions in the reagent composition used in step (c). However, this is a convenient option, also for kit embodiments.

In conclusion, FIGS. 38 and 39 demonstrate the relationship of PEG concentration and selective elution: the higher the PEG concentration during the selective elution, the fewer DNA is eluted during this step. As already noticed during the binding procedure, the lower the PEG concentration, the larger the DNA molecules that remain bound to the magnetic particles.

c) Final Eluates of the Size-Selection Procedure with PEG Concentrations of 15% and 30%

The above findings were also reflected in the finally (purified) eluates obtained from the previous experiments. Desirably, the purified eluates comprise the desired target library DNA, free from impurities such as adapters or adapter dimers. As expected, where the chosen conditions (e.g. the PEG concentration) led to a loss of library DNA during the size-selective elution process, less library DNA was present in the final eluates. Moreover, when the PEG concentration during the selective-elution steps was too high to separate the undesired adapters from the desired library sample during size-selective elution, adapter contaminations were visible in the electropherograms of the eluates. As described herein, the conditions can be adjusted to achieve that the target nucleic acid (e.g. DNA library) is present in the purified eluate, while the undesired non-target nucleic acid (e.g. adapters, adapter dimers) are effectively removed during the process.

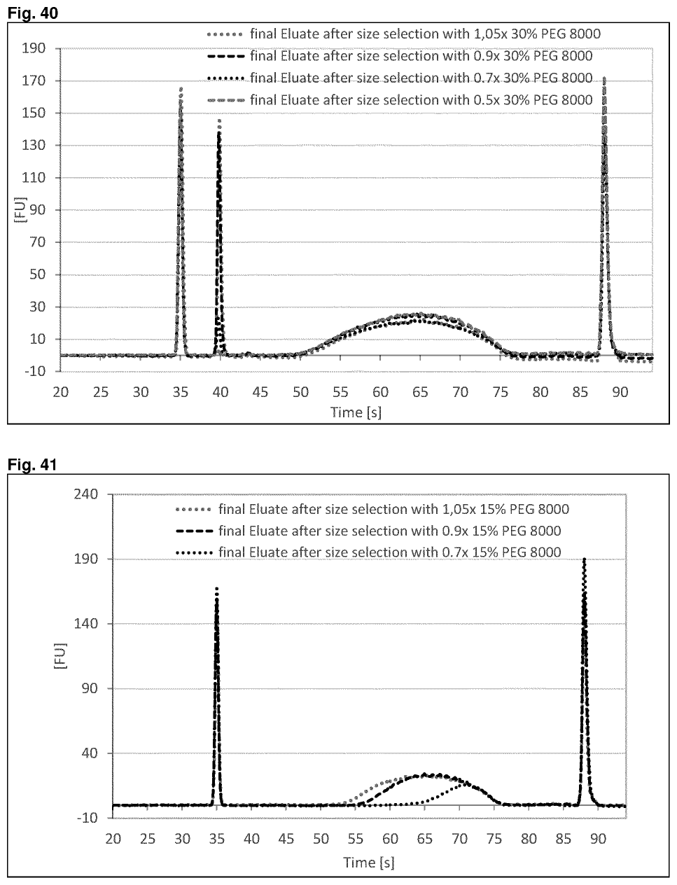

FIG. 40 shows overlaid electropherograms of the purified material (final eluates) after size selective elution with a PEG-buffer that contained 30% (w/v) PEG before being diluted with TE buffer (the dilution factor is shown in the legend). It shows how the conditions for selective elution influence the size of the DNA that remains bound to the magnetic beads and accordingly, is found in the final, purified eluates. If the PEG concentration was high during the selective-elution, adapter dimers were not efficiently eluted from the magnetic particles and can thus still be detected in the final eluate (e.g. dilution factor of 0.7× volume (PEG/TE) and higher). In contrast, if the PEG concentration was lower during the selective-elution step, adapter dimers were readily eluted from the magnetic particles and are not detectable in the eluates as visible by the flat line around 130 bp (40s), see e.g. dilution factor of 0.5× volume PEG/TE.

FIG. 41 shows overlaid electropherograms of the purified material (final eluates) after size selection with PEG-buffer containing 15% (w/v) PEG before being diluted with TE buffer (the dilution factor is shown in the legend). As can be seen, adapter dimers were effectively removed under all conditions. A good library DNA recovery was achieved after size selective elution with 1.05× volumes of PEG-buffer/TE (see also FIG. 39). Library loss increased with higher dilutions of the PEG-buffer and therefore, when lowering the PEG concentration during the selective elution.

In conclusion, the effective removal of the smaller non-target nucleic acid (e.g. adapters) strongly depends on the PEG concentrations that are used for the size-selective elution and furthermore the initial binding step. The PEG concentration used to achieve an efficient removal of the non-target nucleic acids needs to be balanced against a potential loss of target nucleic acid (e.g. library DNA). The above experiments clearly demonstrate using PEG 8000 as an example, that a size-selective elution is possible with a wide range of PEG concentrations in the PEG-buffer. The use of highly concentrated PEG-buffers during binding whereby target as well as non-target nucleic acids are bound to the beads require a higher dilution of the PEG buffers/lower PEG concentration during the size effective elution step in order to elute substantially all the bound adapters during the size selective elution step. With respect to the user-friendliness and robustness of the procedure it is preferred to perform a size selective binding step (a) to achieve that substantially the target nucleic acid is bound, while non-target nucleic acids do not bind or bind to a lesser extent than the target nucleic acids.

### d) Calculated PEG Concentrations During the Various Steps of the Adapter Removal Procedure Using Various PEG-Buffers.

As indicated above, using PEG 8000 and the applied dilution scheme, a PEG concentration of below 15% in the PEG-buffer was too low to reliably bind the library DNA under analysis and follow the procedure without significant target DNA loss. A loss of target DNA could be avoided though by an increase in the binding factor from 1.1× sample volume, whereby the concentration of PEG in the binding mixture is increased. On the other hand, if the PEG concentration was too high in the binding mixture, selective binding was not achieved and adapter dimers readily bound to the magnetic particles during the binding step. In order to remove these adapters from the library DNA during the selective elution, high dilutions of the PEG-buffer were required during these steps. Table 1 displays the final PEG concentration during the size-selective elution steps using various dilutions factors of the PEG-buffer. It gives an overview of calculated PEG 8000 concentrations during the experimental procedure of adapter removal via size-selective elution in the experimental set-up tested. Number in percent indicate the final PEG concentration during the step.

TABLE 1

| | | Size | Size | Size | Size |
|---|---|---|---|---|---|
| | Binding | selective | selective | selective | selective |
| | with factor | elution | elution | elution | elution |
| Reagent | 1.1x | 1.05x | 0.9x | 0.7x | 0.5x |
| 30% | 15.7% | 15.4% | 14.2% | 12.4% | 10% |
| PEG 8000 | | | | | |
| 20% | 10.5% | 10.2% | 9.5% | 8.2% | 6.7% loss |
| PEG 8000 | | | | | of library |
| | | | | | DNA |

| | | | | |
|---|---|---|---|---|
| 15% | 7.9% | 7.7% | 7.1% | 6.2% loss of |
| PEG 8000 | | | | library DNA |

| | |
|---|---|
| 10% | 5.2%; no sufficient binding of DNA to magnetic |
| PEG 8000 | beads due to too low PEG concentration |
| 8% | 4.2%; no sufficient binding of DNA to magnetic |
| PEG 8000 | beads due to too low PEG concentration |

Altogether, in the experimental set-up and for PEG 8000, the calculated concentrations of PEG in the selective-elution steps is preferably in a range between about 8% and 11% to thoroughly elute adapters from the magnetic particles while keeping the library fraction bound to the magnetic particles as indicated by bioanalyzer data (not all traces shown). This seems to be independent of the starting concentration of the PEG-buffer, and hence the amount of adapters that were co-bound to the magnetic particles by higher PEG concentrations during the binding step. Preferred conditions tested are indicated in cursive in Table 1.

Therefore, the use of PEG-buffers with high PEG concentrations for binding require higher dilutions for the size-selective elution process (in order to efficiently remove non-target DNA that was co-bound during the binding step) compared to PEG-buffers with lower concentrations of PEG in order to reach the same final concentration of PEG that facilitates the selective-elution. It is preferred to use a concentration for the PEG-buffer that mediates stable binding of the target nucleic acid (e.g. library-DNA) while leaving non-target nucleic acids (e.g. adapter-dimers) unbound during the initial binding step (see e.g. Table 1). This is advantageous for the robustness of the method.

### Example 6.2: Use of PEG with Different Molecular Weights

Polyethylene glycol (PEG) with varying molecular weight was used to investigate the influence of the molecular weight on the adapter-removal procedure. Since PEG functions as a molecular crowding agent, it is believed that the length of the PEG chains, which corresponds to its molecular weight, would directly influence the concentration of PEG that is needed to successfully perform the method of the invention. The processing of samples and the starting material has been described above. One of the following PEG molecules was used in the PEG-buffer for these experiments: PEG 3000, PEG 8000 or PEG 20000. Apart from 20% PEG, the buffer contained 2.5 M NaCl, 1 mM EDTA, 0.05% Tween 20 (v/v), 10 mM Tris and 3.36 mM HCl.

a) Binding of Starting Material to the Carboxylated Beads Using with PEGs Varying Molecular Weight

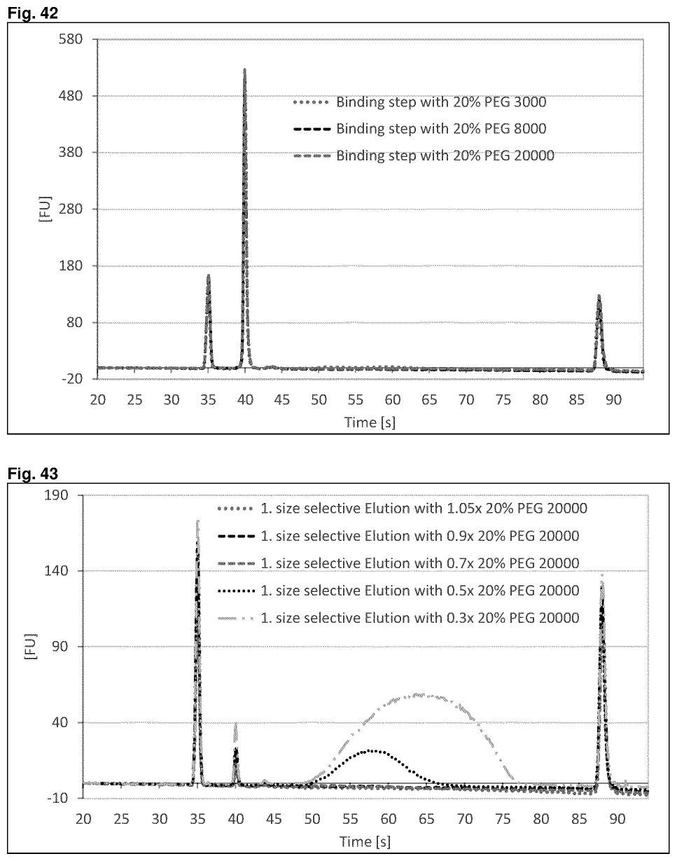

Analogous to step 1 described in section 6.1, a binding factor of 1.1× sample volume was used to bind the library DNA to the magnetic particles. After incubation as outlined in the protocol above, the supernatant was recovered and the unbound DNA fraction was purified using spin-columns. The DNA purified from the supernatant of the binding step was then analyzed using a bioanalyzer. The resulting electropherogram is displayed in FIG. 42.

The electropherogram in FIG. 42 shows that PEG molecules with different molecular weight are capable of binding the target library DNA to magnetic particles, while leaving smaller sized non-target adapter DNA in the supernatant. Under the chosen test conditions there was no substantial difference in the binding effects between high molecular PEG (20.000) and lower molecular PEG (3000) in the binding process. These results suggest that the PEG concentration (20% (w/v) in the PEG-buffer for all tested molecules) rather than the chain length of the tested PEG molecules, played an important role in this binding process. Interestingly, the size-selectivity seems to be similar for all tested PEGs, as the adapter peak that remains unbound to the magnetic particles, appeared similar in all results.

b) Size Selective Elution Using PEG with Different Molecular Weights

After assessment of the influence of the PEG molecular weight in the binding step (a) with the result that the process of binding the target nucleic acids to particles was mostly based on the overall PEG concentration rather than molecular weight of the PEG molecules, the selective elution process (step (c) according to the method of the present disclosure) in the presence of various PEG molecules of different molecular weight was analysed further.

As in previous experiments (see example 6.1), multiple dilutions of PEG-buffer were used to assess the optimal ratio of PEG-buffer to dilution buffer (TE) for the selective-elution process. After the selective elution took place, spin-columns were used to purify the DNAs eluted during the size-selective elution step from the supernatants. FIG. 43 shows the electropherogram of selectively-eluted DNA using PEG 20000 in various dilutions (the factor indicates volumes of PEG-buffer/TE).

During the selective-elution steps using PEG 20000, under most tested conditions mainly the non-target adapter DNA is diluted, while the target library DNA remains bound to the magnetic particles. Only if the PEG-buffer was strongly diluted (factor 0.5× vol PEG-buffer/TE and below) the PEG concentration does not seem sufficient to keep the processed library bound to the magnetic beads and loss of library DNA was observed.

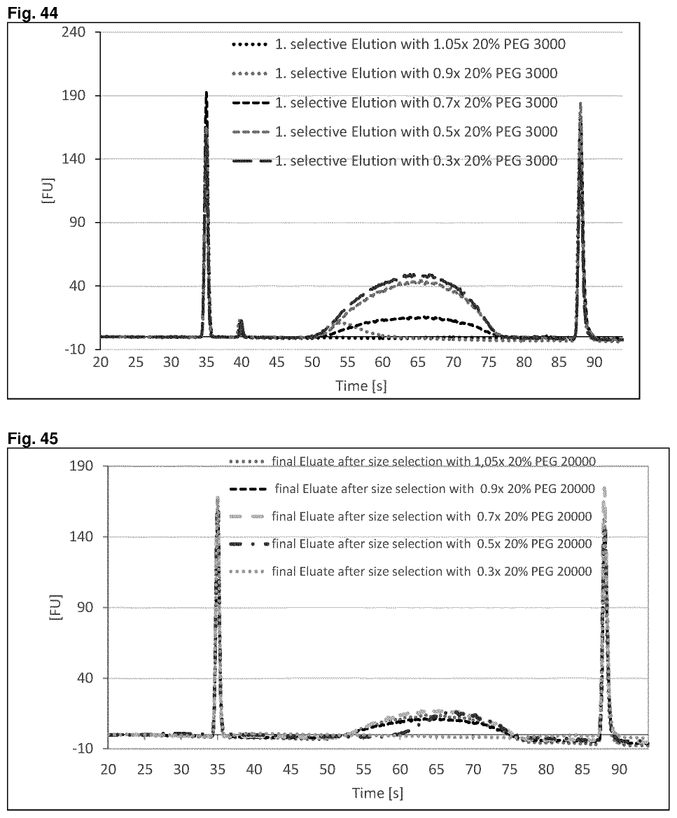

To get an impression of how lower molecular weight PEG performs in the size selective elution process, we repeated the experiment shown in FIG. 43, however, using PEG 3000. The resultant electropherogram is displayed in FIG. 44. Interestingly, the use of lower molecular PEG 3000 results in the elution of library DNA under conditions that were functional when using higher molecular weight PEG 20000 (e.g. 0.7× x volume PEG/TE). When using a dilution factor of 0.5× volume, where first library losses occurred with PEG 20000 (see FIG. 43), the entire library was eluted when PEG 3000 was used (see FIG. 44). When using a dilution factor of 0.9× volumes PEG/TE or higher, no substantial library losses were observed during this step with PEG 3000. From both experiments in combination it can be concluded that the longer PEG chains and hence the higher molecular weight helps to keep the DNA on the magnetic particles during the size-selective elution step. However, this also means that the choice for a specific PEG for a given target nucleic acid must fit the needs of thoroughly binding the target nucleic acid (e.g. library DNA) from the sample material in the binding step, but facilitate selective elution of non-target nucleic acids (adapters) from the particles in the size selective elution process while maintaining binding of the target DNA. It is also within the scope of the present disclosure to use PEGs of different molecular weights in the binding and size selective elution step to adjust and fine-tune the conditions.

c) Elution of Size-Selected DNA Using PEG with Varying Molecular Weight

Completing the adapter-removal workflow by selective elution, in the last step the target DNA that remains bound to the particles during size-selective elution of the non-target DNA is eluted from the beads and the eluate is subsequently analyzed on a bioanalyzer. FIG. 45 shows the DNA fraction that is eluted from the magnetic particles after the protocol has been performed with a PEG-buffer that contained 20% of PEG 20000. The eluates obtained from the size selection workflow using PEG 20000 in the PEG-buffer show that such higher molecular PEG can be used without compromising the results. However, as described previously, the PEG dilution that is used to remove the adapters from the sample during the size-selective elution is important for optimized results. When the dilution factor is too high, library loss occurs during the selective-elution (e.g. in the tested set-up a factor of 0.5× volume PEG/TE of lower). This loss of DNA library is visible in electropherograms of the corresponding eluents (see FIG. 45).

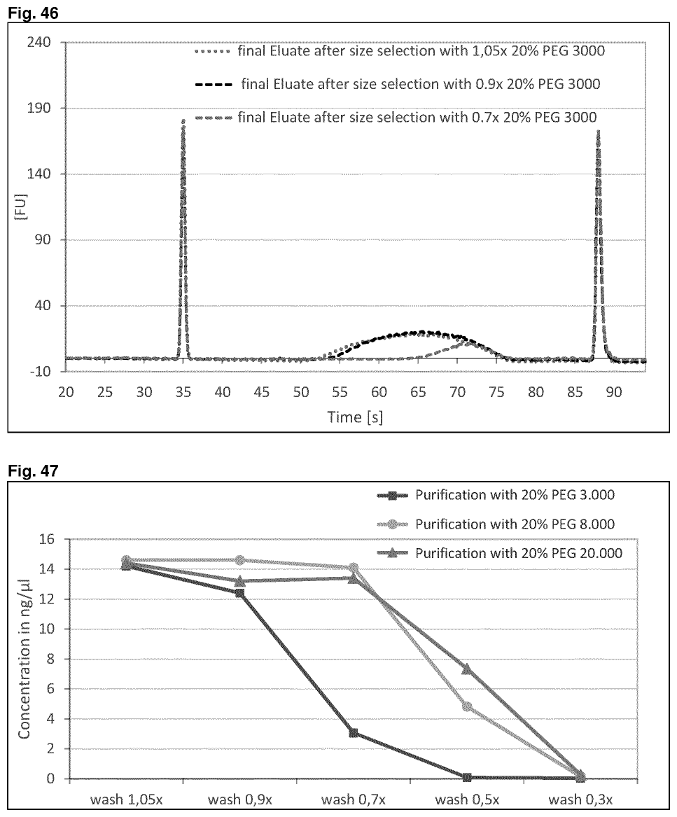

FIG. 46 shows the target DNA fraction that is eluted from the magnetic particles after the protocol has been performed with a PEG-buffer that contained 20% of PEG 3000. The eluates of the size selection workflow using PEG 3000 in the PEG-buffer show that also lower molecular PEG can be used without compromising the results. The adapter-peak that was present in the starting material has been removed from the sample, leaving a prominent library peak in the FIG. 46. First losses were visible after size selective elution with a mixing ratio of 0.7× volume of PEG 3000 (w/v) mixed with 1.0× volume of buffer TE (see FIG. 44). This directly corresponds to the electropherogram of the eluates shown in FIG. 46, where under these conditions less library DNA is present. Therefore, by using a higher concentration of PEG3000 during size selective elution (e.g. 1.05× or 0.9×20% PEG3000), the target DNA (library) could be purified efficiently, while the non-target DNA (adapters) were efficiently depleted during the process.

d) Conclusion of Size-Selection of DNA Using PEG with Varying Chain lengthA

successful depletion of adapter dimers is possible using polyethylene glycol (PEG) of varying molecular weight. In this experiment the performance of three different PEG molecules was tested: PEG 3000, 8000 and 20000. All of these tested PEGs variations mediated the binding of library DNA to the magnetic particles. Moreover, a prominent size-selectivity could be achieved with all PEG molecules used, as adapter dimers remained in the supernatant of the binding step and were not bound to the beads along with the library DNA. The main difference of the various sized PEG molecules becomes visible during the size-selective elution. Here, longer PEG molecules seem to facilitate maintaining binding of (library) DNA to the magnetic particles during the size selective elution process. Only in very high dilution, library is lost during the selective-elution process. When using a PEG with lower molecular weight, the point of selectively eluting library is already reached in lower PEG dilutions, indicating that the length of the PEG (molecular weight) influences that DNA binding to magnetic particles is maintained during size selective elution. A conclusive plot of the yields following the procedures is shown in FIG. 47.

### 6.3. Further Conclusions

The results show the broad applicability of the methods disclosed herein regarding the PEG concentration and the molecular weight of the used PEG. To efficiently remove non-target nucleic acids but avoid loss of target nucleic acids, the methods allow to advantageously adjust the parameters during binding and selective elution based on the principles described herein. By adjusting the parameters, various sizes of non-target and target nucleic acids can be effectively processed by the present method. For instance, as shown in the above experiments, a mixture of library A and library B (300 to 1800 bp and 120 to 130 bp, respectively) can be efficiently separated, i.e. library A can be purified, by selectively eluting with a diluted PEG-buffer (reagent) comprising e.g. 8 to 11% PEG 8000 (w/v). Optimal ranges of PEG concentrations may be determined for other sizes/size ranges of target and non-target nucleic acids. For example, a library having a large size will effectively bind to the solid phase at a lower PEG concentration and/or molecular weight. Therefore, also lower concentrations or lower molecular weights of PEG are useful in such settings. At the same time, small non-target nucleic acids, including adapter dimers, can be removed at such a PEG concentration/molecular weight, while even larger contaminants may be removed, if present. Vice versa, if a library having a small size of the target nucleic acids is applied, the PEG concentration and/or molecular weight should be sufficiently high in the binding step in order to bind the library to the solid phase. The PEG-concentration in the reagent composition of step (c) can be adjusted such that the adapter dimers are selectively eluted while the library remains bound to the solid phase.

As shown above, the method according to the present disclosure allows such adjustment and can advantageously isolate target nucleic acids, while non-target nucleic acids are removed. Adjustment to different sizes or size ranges of target and non-target nucleic acids can be achieved by applying the various parameters as described herein; preferably by the PEG concentration, PEG molecular weight, and/or dilution factor. For instance, selective elution without a substantial loss of target nucleic acids (e.g. library) may be achieved by providing 7 to 11% PEG 20000, 8 to 11% PEG 8000 or 9 to 11% PEG 3000 in the reagent composition of step (c). Hence, these factors can be adjusted e.g. as follows:
selecting a higher concentration of PEG in the binding step (e.g. 30% (w/v)) necessitates a higher dilution factor for selective elution if the reagent composition (c) is prepared from the binding reagent (e.g. 0.5×);
selecting a PEG having a lower molecular weight (e.g. PEG 3000) necessitates higher PEG concentrations for selective elution to prevent loss of target nucleic acids (e.g. 9 to 11%);
selecting a low dilution factor preferably is used in combination with size selectivity in the binding step (e.g., 20% PEG 8000, 1.1× binding and 20% PEG 8000 1.05× selective elution); or
selecting target nucleic acids having a small size necessitates stronger binding conditions (e.g., 30% PEG 8000, 1.1×).

1. A poly(alkylene oxide) polymer based size selective nucleic acid enrichment method for enriching target nucleic acid molecules from a nucleic acid containing sample which comprises target nucleic acid molecules and non-target nucleic acid molecules, wherein the target nucleic acid molecules are longer than the non-target nucleic acid molecules, the method comprising
(a) preparing a binding mixture comprising
the nucleic acid containing sample,
a poly(alkylene oxide) polymer and
a salt and binding nucleic acid molecules to a solid phase which comprises a functional group, preferably carboxylated magnetic particles, wherein the bound nucleic acid molecules comprise target nucleic acid molecules;
(b) preferably separating the solid phase with the bound nucleic acid molecules from the remaining sample;
(c) contacting the solid phase with the bound nucleic acid molecules at least once with a reagent composition comprising a poly(alkylene oxide) polymer and a salt to selectively elute non-target nucleic acid molecules, wherein the concentration (w/v) of the poly(alkylene oxide) polymer in the reagent composition of step (c) is lower than the concentration (w/v) of the poly(alkylene oxide) polymer in the binding mixture of step (a);
(d) optionally washing the bound target nucleic acid molecules; and
(e) eluting the bound target nucleic acid molecules from the solid phase.

2. The method according to claim 1, wherein in step (a), target nucleic acid molecules having a size above a cut-off value bind to the solid phase and wherein under the used binding conditions non-target nucleic acid molecules having a size below the cut-off value do not bind to the solid phase or bind to a lesser extent to the solid phase compared to the longer target nucleic acid molecules.

3. The method according to claim 1 or 2, wherein the solid phase has one or more of the following characteristics:
a) the solid phase comprises ionic groups, preferably acidic groups;
b) the solid phase comprises carboxyl groups;
c) the solid phase is provided by particles, preferably magnetic particles; and/or
d) the solid phase is provided by carboxylated magnetic particles, wherein optionally, the magnetic particles are paramagnetic.

4. The method according to any one of claims 1 to 3, wherein step (c) is performed two or more times.

5. The method according to one or more of claims 1 to 4, for enriching adapter ligated DNA molecules as target DNA molecules from a DNA containing sample which is an adapter ligation sample and for removing adapter monomers and adapter-adapter ligation products as non-target DNA molecules, wherein adapter ligated DNA molecules are separated from the smaller unligated adapter monomers and adapter-adapter ligation products based on the larger size of the adapter ligated DNA molecules, wherein preferably, adapter monomers and adapter-adapter ligation products having a size of 140 bp or less are removed in the course of the method.

6. The method according to one or more of claims 1 to 5, having one or more of the following characteristics:
a) the poly(alkylene oxide) polymer is a polyethylene glycol;
b) the poly(alkylene oxide) polymer, which preferably is polyethylene glycol, has a molecular weight that lies in a range of 2000 to 40000, preferably in a range selected from 3000 to 30000 and 5000 to 25000, such as in a range of 6000 to 20000;
c) a poly(alkylene oxide) polymer, preferably a polyethylene glycol, of the same molecular weight is used in step (a) and in step (c); or a poly(alkylene oxide) polymer, preferably a polyethylene glycol, of differing molecular weight is used in step (a) and in step (c), wherein the molecular weight of the polymer that is used in step (c) is higher or lower, preferably higher, than the molecular weight of the polymer that is used in step (a);
d) the binding mixture comprises the poly(alkylene oxide) polymer, which preferably is a polyethylene glycol, in a concentration of at least 5% (w/v), such as at least 6%, at least 7%, at least 8% or at least 9%; and/or
e) the binding mixture comprises the poly(alkylene oxide) polymer, which preferably is a polyethylene glycol, in a concentration that lies in a range of 5% to 30% (w/v), preferably in a range selected from 6% to 25% (w/v), 7% to 20% (w/v) and 7.5% to 15% (w/v) and more preferably in a range of 8% to 15% or 8.5% to 13% (w/v).

7. The method according to one or more of claims 1 to 6, having one or more of the following characteristics:
a) the salt is a non-chaotropic salt;
b) the salt is a monovalent salt;
c) the salt is an alkali metal salt, preferably an alkali metal halide;
d) the salt is selected from sodium chloride, potassium chloride, lithium chloride and cesium chloride, more preferably the salt is sodium chloride;
e) the salt is present in the binding mixture in a concentration of ≥350 mM, optionally selected from ≥500 mM, ≥700 mM, ≥800 mM, ≥900 mM and ≥1M; and/or
f) the salt is present in the binding mixture in a concentration that lies in a range of 350 mM to 3.5M, optionally selected from 500 mM to 3M, 700 mM to 2.5M, 800 mM to 2.25M, 900 mM to 2M and 1M to 1.75M.

8. The method according to one or more of claims 1 to 7, wherein step (a) comprises adding a binding reagent to the nucleic acid containing sample to prepare the binding mixture, wherein the binding reagent comprises the poly(alkylene oxide) polymer, preferably a polyethylene glycol, and the salt.

9. The method according to claim 8, having one or more of the following characteristics:
a) the binding conditions are exclusively established by contacting the binding reagent with the nucleic acid containing sample;
b) the binding reagent comprises the salt, which preferably is an alkali metal salt, in a concentration that lies in a range of 0.5M to 5M, preferably in a range selected from 0.7M to 4.5M, 1M to 4.25M, 1.25M to 4M, 1.5M to 3.75M and 1.75M to 3.5M;
c) the binding reagent comprises the poly(alkylene oxide) polymer, which preferably is a polyethylene glycol, in a concentration that lies in a range of 7% to 50% (w/v), preferably in a range selected from 10% to 45%, 12% to 40% and 15% to 35% (w/v);
d) the binding reagent is selected from the group of the following binding reagents:
(aa) a binding reagent comprising
a polyethylene glycol having a molecular weight that lies in a range of 2000 to 40000, preferably in a range selected from 3000 to 30000, 5000 to 25000 and 6000 to 25000;
an alkali metal salt in a concentration that lies in a range of 0.5M to 5M, preferably selected from 0.7M to 4.5M, 1M to 4.25M, 1.25M to 4M, 1.5M to 3.75M and 1.75M to 3.5M;
(bb) a binding reagent comprising
a polyethylene glycol having a molecular weight that lies in a range of 3000 to 30000, preferably in a range selected from 5000 to 25000 and 6000 to 20000;
an alkali metal salt in a concentration that lies in a range of 1M to 4M, preferably selected from 1.5M to 3.75M and 2M to 3.5M;
(cc) a binding reagent comprising
a polyethylene glycol having a molecular weight that lies in a range of 5000 to 25000, optionally 6000 to 20000, in a concentration that lies in a range of 7% to 45% (w/v), preferably selected from 10% to 40% (w/v), 12% to 35% (w/v) and 15% to 30% (w/v);
an alkali metal salt in a concentration that lies in a range of 1M to 4M, preferably selected from 1.5M to 3.75M and 2M to 3.5M;
and/or
(dd) a binding reagent comprising
a polyethylene glycol having a molecular weight that lies in a range of 5000 to 25000, optionally 6000 to 20000, in a concentration that lies in a range of 10% to 40% (w/v), preferably 15% to 35% (w/v);
an alkali metal salt selected from sodium chloride and potassium chloride in a concentration selected from 1.5M to 3.5M and 2M to 3M; and/or
e) the binding reagent comprises the solid phase which is provided by particles, preferably magnetic particles.

10. The method according to one or more of claims 1 to 9, wherein the reagent composition of step (c), which optionally is provided by a single reagent (c), has one or more of the following characteristics:
a) it comprises the poly(alkylene oxide) polymer, which preferably is a polyethylene glycol, in a concentration of at least 5% (w/v), preferably at least 6%, at least 7%, at least 8%, at least 9% or at least 10%;
b) it comprises the poly(alkylene oxide) polymer, which preferably is a polyethylene glycol, in a concentration that lies in a range of 5% to 20% (w/v), preferably in a range selected from 6% to 18%, 6.5% to 15%, 7% to 13%, 7.5% to 12% and 8% to 11% (w/v);
c) it comprises the salt, which preferably is an alkali metal salt, more preferably sodium chloride, in a concentration of 350 mM, preferably selected from 500 mM, 700 mM ≥800 mM, ≥900 mM and ≥1M;
d) it comprises the salt, which preferably is an alkali metal salt, more preferably sodium chloride, in a concentration that lies in a range of 350 mM to 3.5M, preferably in a range selected from 500 mM to 3M, 700 mM to 2.5M, 800 mM to 2.25M, 900 mM to 2M and 1M to 1.75M; and/or
e) the reagent composition of step (c) is selected from the group of the following reagents:
(aa) a reagent composition (c) comprising
a polyethylene glycol having a molecular weight that lies in a range of 3000 to 40000, preferably in a range selected from 3000 to 30000, 5000 to 25000, 6000 to 25000, and 8000 to 20000; and
an alkali metal salt in a concentration that lies in a range of 350 mM to 3.5M, preferably in a range selected from 500 mM to 3M, 700 mM to 2.5M, 800 mM to 2.25M, 900 mM to 2M and 1M to 1.75M;
(bb) a reagent composition (c) comprising
a polyethylene glycol having a molecular weight that lies in a range selected from 5000 to 25000, such as in a range of 6000 to 25000 or 8000 to 20000; and
an alkali metal salt in a concentration that lies in a range of 500 mM to 2.5M, preferably in a range selected from 700 mM to 2.25M, 900 mM to 2M and 1M to 1.75M;
(cc) a reagent composition (c) comprising
a polyethylene glycol having a molecular weight that lies in a range of 5000 to 25000, such as in a range of 6000 to 25000 or 8000 to 20000, in a concentration that lies in a range of 6% to 20% (w/v), preferably in a range selected from 6.5% to 15%, 7% to 13% and 7.5% to 12% (w/v), and
an alkali metal salt in a concentration of 500 mM to 2.5M, preferably selected from 700 mM to 2.25M, 900 mM to 2M and 1M to 1.75M; and
(dd) a reagent composition (c) comprising
a polyethylene glycol having a molecular weight that lies in a range of 5000 to 25000, such as in a range of 6000 to 25000 or 8000 to 20000, in a concentration that lies in a range of 6.5% to 15% (w/v), such as 7.0% to 13% and 7.5% to 12% (w/v), and
an alkali metal salt in a concentration selected from 800 mM to 2M, 900 mM to 1.75M and 1M to 1.5M.

11. The method according to one or more of claims 8 to 10, wherein
a) the concentration (w/v) of the poly(alkylene oxide) polymer in the binding reagent that is added in step (a) is higher than the concentration (w/v) of the poly(alkylene oxide) polymer in the reagent composition of step (c);
and/or
b) wherein the concentration of the salt in the binding reagent that is added in step (a) is higher than the concentration of the salt in the reagent composition of step (c).

12. The method according to one or more of claims 8 to 11, comprising providing the reagent composition of step (c) by mixing the binding reagent with a dilution solution, optionally wherein the binding reagent is a binding reagent having one or more characteristics as defined in claim 8 or 9 and the provided reagent composition of step (c) has one or more characteristics as defined in claim 10 a) to e).

13. The method according to one or more of claims 1 to 12, wherein the target nucleic acid is DNA and wherein the nucleic acid containing sample has one or more of the following characteristics:
i) it is a sample of extracted DNA or extracted DNA that has been further processed, e.g. by shearing or by way of an enzymatic reaction;
ii) the contained nucleic acids comprise or substantially consists of linear, double-stranded DNA molecules;
iii) it was obtained after an enzymatic reaction, preferably selected from amplification reactions, ligase reactions, in particular adapter ligation reactions, and polynucleotide, e.g. poly A, tailing reactions;
iv) the contained nucleic acids comprise or substantially consist of fragmented DNA;
v) the contained nucleic acids comprise or substantially consist of linear, blunt-ended DNA fragments of different sizes;
vi) it comprises amplification products, preferably PCR products;
vii) the DNA containing sample is an adapter ligation sample that was obtained as a result of an adapter ligation step, preferably during preparation of a sequencing library; and/or
viii) it is an adapter ligation sample which comprises (i) double-stranded DNA molecules that are flanked 5′ and/or 3′ by adapters, (ii) adapter monomers and (iii) adapter-adapter ligation products.

14. The method according to one or more of claims 1 to 13, wherein the method comprises
(a) preparing a binding mixture comprising
the nucleic acid containing sample,
a poly(alkylene oxide) polymer having a molecular weight that lies in a range of 3000 to 25000, such as 6000 to 20000,
wherein preferably, the poly(alkylene oxide) polymer is a polyethylene glycol and
a salt, wherein the salt is an alkali metal salt, preferably selected from sodium chloride and potassium chloride,
and binding nucleic acid molecules to the solid phase, wherein the bound nucleic acid molecules comprise target nucleic acid molecules,
optionally wherein under the used binding conditions the smaller non-target nucleic acid molecules having a length below a cut-off value do not bind to the solid phase or bind to a lesser extent to the solid phase compared to the longer target nucleic acid molecules;
(b) separating the solid phase with the bound nucleic acid molecules from the remaining sample;
(c) contacting the solid phase with the bound nucleic acid molecules at least once with a reagent composition comprising
a poly(alkylene oxide) polymer having a molecular weight that lies in a range of 3000 to 25000, preferably 5000 to 25000, such as 6000 to 20000, wherein preferably, the poly(alkylene oxide) polymer is a polyethylene glycol, and
a salt, wherein the salt is an alkali metal salt, preferably selected from sodium chloride and potassium chloride,
to selectively elute non-target nucleic acid molecules having a size below a cut-off, wherein the concentration (w/v) of the poly(alkylene oxide) polymer in the reagent composition of step (c) is lower than the concentration (w/v) of the poly(alkylene oxide) polymer in the binding mixture of step (a);
(d) optionally washing the bound target nucleic acid molecules; and
(e) eluting the bound target nucleic acid molecules from the solid phase.

15. The method according to one or more of claims 5 to 14, wherein the method comprises
(a) preparing a binding mixture comprising
the adapter ligation sample,
a poly(alkylene oxide) polymer, preferably a polyethylene glycol, and
a salt
and binding nucleic acid molecules to the solid phase, wherein the bound nucleic acid molecules comprise adapter ligated DNA molecules,
optionally wherein under the used binding conditions the smaller adapter monomers and adapter-adapter ligation products having a length below a cut-off value do not bind to the solid phase or bind to a lesser extent to the solid phase compared to the longer adapter ligated DNA molecules;
(b) separating the solid phase with the bound DNA molecules from the remaining sample;
(c) contacting the solid phase with the bound DNA molecules at least once with a reagent composition comprising a poly(alkylene oxide) polymer, preferably a polyethylene glycol, and a salt to selectively elute adapter monomers and adapter-adapter ligation products, wherein the concentration (w/v) of the poly(alkylene oxide) polymer in the reagent composition of step (c) is lower than the concentration (w/v) of the poly(alkylene oxide) polymer in the binding mixture of step (a);
(d) optionally washing the bound adapter ligated DNA molecules; and
(e) eluting the bound adapter ligated DNA molecules from the solid phase.

16. The method according to claim 15, wherein the method comprises
(a) contacting a binding reagent that comprises a poly(alkylene oxide) polymer and a salt with a DNA containing sample which is an adapter ligation sample, thereby preparing a binding mixture comprising
the adapter ligation sample,
the poly(alkylene oxide) polymer, preferably a polyethylene glycol, in a concentration of at least 7% (w/v), preferably at least 8%, wherein the poly(alkylene oxide) polymer has a molecular weight that lies in a range of 3000 to 30000, preferably in a range of 5000 to 25000, such as 6000 to 20000, and
the salt in a concentration of at least ≥500 mM, preferably at least 800 mM, wherein the salt is a non-chaotropic salt, preferably an alkali metal salt, more preferably selected from sodium chloride and potassium chloride,
and binding nucleic acid molecules to the solid phase, wherein the bound nucleic acid molecules comprise adapter ligated DNA molecules and wherein the solid phase is provided by carboxylated magnetic particles,
optionally wherein under the used binding conditions the smaller adapter monomers and adapter-adapter ligation products having a length below a cut-off value do not bind to the solid phase or bind to a lesser extent to the solid phase compared to the longer adapter ligated DNA molecules;
(b) separating the solid phase with the bound DNA molecules from the remaining sample;
(c) contacting the solid phase with the bound DNA molecules at least once with a reagent composition comprising
a poly(alkylene oxide) polymer having a molecular weight that lies in a range of 3000 to 30000, preferably in a range of 5000 to 25000, such as 6000 to 20000, wherein preferably, the poly(alkylene oxide) polymer is a polyethylene glycol, and
a salt, wherein the salt is a non-chaotropic salt, preferably an alkali metal salt, more preferably selected from sodium chloride and potassium chloride,
to selectively elute adapter monomers and adapter-adapter ligation products, wherein the concentration (w/v) of the poly(alkylene oxide) polymer in the reagent composition of step (c) is lower than the concentration (w/v) of the poly(alkylene oxide) polymer in the binding mixture of step (a),
optionally wherein the reagent composition of step (c) is provided by mixing the binding reagent with a dilution solution, optionally wherein the binding reagent is a binding reagent as defined in claim 8 or 9 and the provided reagent composition of step c) is as defined in claim 10;
(d) optionally washing the bound adapter ligated DNA molecules; and
(e) eluting the bound adapter ligated DNA molecules from the carboxylated magnetic particles.

17. The method according to any one of claims 5 to 16 when depending on claim 5, wherein the method comprises
(a) contacting a binding reagent as defined in claim 9 d) with a DNA containing sample which is an adapter ligation sample, thereby preparing a binding mixture comprising
the adapter ligation sample,
the polyethylene glycol in a concentration of at least 7% (w/v), preferably at least 8%, and
the salt in a concentration of at least 750 mM, preferably at least 800 mM, more preferably at least 900 mM,
and binding adapter ligated DNA molecules to the solid phase which is provided by carboxylated magnetic particles, wherein under the used binding conditions the smaller adapter monomers and adapter-adapter ligation products do not bind to the solid phase or bind to a lesser extent to the solid phase compared to the longer adapter ligated DNA molecules;
(b) separating the solid phase with the bound DNA molecules from the remaining sample;
(c) providing a reagent composition as defined in claim 10 e) by mixing the binding reagent used in step (a) with a dilution solution and contacting the separated solid phase comprising the bound DNA molecules at least once with the provided reagent composition to selectively elute adapter monomers and adapter-adapter ligation products from the solid phase, wherein the concentration (w/v) of the polyethylene glycol in the reagent composition of step (c) is lower than the concentration (w/v) of the polyethylene glycol in the binding mixture of step (a);
(d) optionally washing the bound adapter ligated DNA molecules; and
(e) eluting the bound adapter ligated DNA molecules from the carboxylated magnetic particles.

18. The method according to any one of claims 1 to 17, characterized by the following features:
the method is for enriching adapter ligated DNA molecules as target DNA molecules from a DNA containing sample which is an adapter ligation sample and for removing adapter monomers and adapter-adapter ligation products as non-target DNA molecules, wherein adapter ligated DNA molecules are separated from the smaller unligated adapter monomers and adapter-adapter ligation products based on the larger size of the adapter ligated DNA molecules,
the nucleic acid containing sample it is an adapter ligation sample which comprises (i) double-stranded DNA molecules that are flanked 5′ and/or 3′ by adapters, (ii) adapter monomers and (iii) adapter-adapter ligation products,
step (a) comprises adding a binding reagent to the nucleic acid containing sample to prepare the binding mixture, wherein the binding reagent comprises the poly(alkylene oxide) polymer, preferably a polyethylene glycol, and the salt and wherein in step (a), target nucleic acid molecules having a size above the cut-off value bind to the solid phase and wherein under the used binding conditions non-target nucleic acid molecules having a size below the cut-off value do not bind to the solid phase or bind to a lesser extent to the solid phase compared to the longer target nucleic acid molecules,
steps (b) is performed, and
wherein adapter monomers and adapter-adapter ligation products having a size of 140 bp or less are removed in the course of the method.

19. The method according to claim 18, wherein the concentration (w/v) of the poly(alkylene oxide) polymer in the binding reagent that is added in step (a) is higher than the concentration (w/v) of the poly(alkylene oxide) polymer in the reagent composition of step (c) and wherein the concentration of the salt in the binding reagent that is added in step (a) is higher than the concentration of the salt in the reagent composition of step (c).

20. The method according to claim 19, wherein
the binding reagent comprises
a polyethylene glycol having a molecular weight that lies in a range of 5000 to 25000, optionally 6000 to 20000, in a concentration that lies in a range of 10% to 40% (w/v), 12% to 35% (w/v) or 15% to 30% (w/v);
an alkali metal salt in a concentration that lies in a range of 1M to 4M, preferably selected from 1.5M to 3.75M and 2M to 3.5M; and
the reagent composition (c) comprises
a polyethylene glycol having a molecular weight that lies in a range of 5000 to 25000, such as in a range of 6000 to 20000, in a concentration that lies in a range of 6% to 20% (w/v), preferably in a range selected from 6.5% to 15%, 7% to 13% and 7.5% to 12% (w/v), and
an alkali metal salt in a concentration of 500 mM to 2.5M, preferably selected from 700 mM to 2.25M, 900 mM to 2M and 1M to 1.75M.

21. The method according to any one of claims 18 to 20, comprising providing the reagent composition of step (c) by mixing the binding reagent with a dilution solution, optionally wherein the binding reagent is a binding reagent having one or more characteristics as defined in claim 8 or 9 and the provided reagent composition of step (c) has one or more characteristics as defined in claim 10 a) to e).

22. A method for preparing a sequencing library, wherein said method comprises
A) optionally fragmenting DNA and optionally end repairing DNA fragments to provide a sample comprising blunt end DNA fragments of different sizes;
B) performing an adapter ligation step to provide a sample comprising double-stranded DNA molecules that are flanked 5′ and/or 3′ by adapters,
C) separating adapter ligated double-stranded DNA molecules as target DNA molecules from unligated adapter monomers and adapter-adapter ligation products as non-target DNA molecules based on the larger size of the adapter ligated double stranded target DNA molecules using the method according to any one of claims 1 to 21;
D) optionally amplifying adapter ligated DNA molecules.

23. A kit for the size selective enrichment of target nucleic acid molecules having a size above a desired cut-off value from a nucleic acid containing sample, comprising
(a) a binding reagent comprising at least one poly(alkylene oxide) polymer and at least one salt;
(b) magnetic particles for binding target nucleic acid molecules in the presence of the binding reagent (a);
(c) a reagent comprising at least one poly(alkylene oxide) polymer and at least one salt and/or a dilution reagent for preparing the reagent (c) by combining the dilution reagent with the binding reagent;
(d) optionally at least one washing solution; and
(e) an elution solution,
wherein the concentration of the poly(alkylene oxide) polymer in the binding reagent (a) is higher than the concentration of the poly(alkylene oxide) polymer in the reagent (c).

24. The kit according to claim 23, having one or more of the following characteristics:
a) wherein the concentration of the salt in the binding reagent (a) is higher than the concentration of the salt in the reagent (c);
b) the binding reagent (a) has one or more characteristics as defined in claim 9;
c) reagent (c) has one or more of the characteristics as defined in claim 10;
d) the solid phase has one or more of the characteristics as defined in claim 3; and/or
e) the solid phase is comprised in binding reagent (a), wherein preferably, the solid phase is provided by magnetic particles, more preferably by carboxylated magnetic particles.
